(19) United States
(12) Patent Application Publication
(10) Pub. No.: US 2023/0181746 A1
(43) Pub. Date: Jun. 15, 2023

(54) BENZOYLHYDRAZIDE-DERIVED HDAC DEGRADERS AS THERAPEUTICS FOR TREATING CANCER AND OTHER HUMAN DISEASES

(71) Applicant: University of Florida Research Foundation, Inc., Gainesville, FL (US)

(72) Inventors: Daiqing LIAO, Gainesville, FL (US); Yufeng XIAO, Gainesville, FL (US); Xuan ZHANG, Gainesville, FL (US); Guangrong ZHENG, Gainesville, FL (US)

(21) Appl. No.: 17/925,776

(22) PCT Filed: May 17, 2021
(86) PCT No.: PCT/US2021/032703

§ 371 Date: Nov. 16, 2022

Related U.S. Application Data
(60) Provisional application No. 63/026,801, filed on May 19, 2020.

(51) Int. Cl. A61K 47/55; A61P 35/00; A61K 47/60

(52) U.S. Cl. CPC A61K 47/55; A61P 35/00; A61K 47/60

(57) ABSTRACT

In one aspect, the disclosure relates to benzoylhydrazide-derived PROTACs that are highly effective at degrading HDAC3 and that are also capable of targeting, to a lesser extent, other HDAC isoforms, methods of making same, pharmaceutical compositions comprising same, and methods of treating cancers including hematologic cancers, breast cancer, other malignancies, and other serious diseases involving aberrant HDAC activity using the same. This abstract is intended as a scanning tool for purposes of searching in the particular art and is not intended to be limiting of the present disclosure.

### CROSS-REFERENCE TO RELATED APPLICATIONS

This application claims the benefit of and priority to co-pending U.S. Provisional Patent Application No. 63/026,801, filed on May 19, 2020, the contents of which are incorporated by reference herein in their entireties.

### STATEMENT REGARDING FEDERALLY SPONSORED RESEARCH OR DEVELOPMENT

This invention was made in whole or in part from funding received under agreement number AGR DTD 01-30-2015, received from UF Health Shands Hospital, and under grant numbers 20K07, 6JK03 and 6BC03, received from the Florida Department of Health.

### BACKGROUND

Histone deacetylases (HDACs) and histone acetyltransferases (HAT) are critical regulators of chromatin accessibility during transcription, replication, recombination, and repair. In human cells, there are eleven isoforms of zinc-dependent HDACs, which can be divided into four phylogenetic groups: class I (HDACs 1-3, and 8), class IIa (HDACs 4, 5, 7, and 9), class IIb (HDACs 6 and 10), and class IV (HDAC 11). HDACs are commonly overexpressed in various cancer types including hematologic and solid malignancies. Among them, class I HDACs are critical to activating oncogenes underlying tumorigenesis, disease progression and treatment resistance. Recent studies have shown that HDACs 1-3 are important for oncogene expression regulated by super enhancers in breast and other cancer types. Dysregulation of HDACs is also implicated in the pathogenesis of several other human diseases including metabolic disorders such as type 2 diabetes, adipose tissue inflammation, excessive hepatic lipid accumulation, lipodystrophy, and insulin resistance; neurodegenerative and neurological diseases, inflammatory disorders such as rheumatoid arthritis, asthma, chronic obstructive pulmonary disease, cystic fibroses, acute respiratory distress syndrome, and interstitial fibrosis; kidney disease; infectious diseases including influenza and pneumonia; and cardiovascular diseases and their complications including heart disease, stroke, and the like.

During the past three decades, a number of HDAC inhibitors (HDACi) with different chemical scaffolds have been developed, and many of them have been evaluated in preclinical and clinical studies for their anticancer activities. So far, four HDACi have been approved by the United States Food and Drug Administration for treating lymphomas, leukemias, and multiple myelomas. A typical HDACi consists of a zinc binding group (ZBG), a surface-recognition cap group, and an appropriate linker. Among those components, ZBG plays an important role in subtype selectivity. Most hydroxamate-derived HDACi are pan-HDACi with limited isoform selectivity whereas benzamide-based HDACi prefer to bind to class I HDACs. Fine-tuned surface-recognition cap groups can improve isoform selectivity, as well. Different types of HDACi have been tested clinically, but none of them have achieved clinical success for treating solid tumors as a single agent, which is probably due to their ineffectively low concentrations in tumor tissue. Importantly, dose-limiting adverse effects such as cardiac toxicity associated with hERG K⁺ channel activation are hindering their progress in the clinic. Increased HDAC isoform selectivity and novel strategies to abolish HDAC activity could lead to more effective drug candidates to achieve clinical success.

Proteolysis targeting chimera (PROTAC) has emerged as a revolutionary technology in drug discovery. PROTACs possess several advantages over conventional inhibitors such as high potency, extended duration of action, and potential tissue/cell type selectivity. Moreover, due to their unique mechanism of action (MOA) via the formation of ternary complexes, specific proteasomal degradation of protein of interest (POI) can be achieved even when recruiting a “dirty” warhead. Recently, several attempts have been made to degrade HDACs using the PROTAC approach. However, by conjugating either pan-HDACi or HDAC6 inhibitors as warheads, PROTACs 1-5 can only degrade HDAC 6 (FIG. 1). By employing a benzamide-based HDAC binder as the warhead, PROTAC 6 that degraded class I HDACs was developed. Nevertheless, weak HDAC3 degradation was observed in comparison to degradation of HDAC1 and HDAC2, probably because the warhead CI-994 more favorably binds to HDAC1 and HDAC2 instead of HDAC3.

Despite advances in research targeting HDAC degradation, there is still a scarcity of compounds that are potent, efficacious, and selective inhibitors and/or PROTACs of HDAC3 and other HDACs, that are also effective in the treatment of blood cancers, solid malignancies, metabolic disorders, neurological and neurodegenerative disorders, and inflammatory disorders associated with aberrant HDAC activity. These needs and other needs are satisfied by the present disclosure.

### SUMMARY

In accordance with the purpose(s) of the present disclosure, as embodied and broadly described herein, the disclosure, in one aspect, relates to benzoylhydrazide-derived PROTACs that are highly effective at degrading HDAC3 and that are also capable of targeting, to a lesser extent, other HDAC isoforms, methods of making same, pharmaceutical compositions comprising same, and methods of treating cancers including hematologic cancers, breast cancer, other malignancies, and other serious diseases involving aberrant HDAC activity using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows reported PROTACs targeting HDAC6 or Class I HDACs.

FIGS. 2A-2C show the rational design of HDAC3 degraders. FIG. 2A shows the chemical structure of lead compound SR-3558 that selectively binds to class I HDACs. FIG. 2B shows a molecular docking study using Autodock Vina revealed the lowest binding energy pose for SR-3558 with HDAC3 {PDB: 4A69}. FIG. 2C shows the chemical structures of CRBN- and VHL-based PROTACs derived from SR-3558.

FIGS. 3A-3D show that XZ9002 induces potent HDAC3 degradation and, to a lesser extent, degradation of HDAC1 and HDAC2 at a high concentration. FIG. 3A: Western blot showing HDAC protein levels in MDA-MB-468 cells treated with the indicated concentration of XZ9002 for 14 h. FIG. 3B: pretreatment with 1 μM MG132, or 10 μM VHL032 for 1 h blocked the degradation of HDAC3 by XZ9002 {125 nM}. XZ9002-NC, the negative control of XZ9002 that does not bind to VHL, cannot degrade HDAC3. NC: XZ9002-NC. FIG. 3C: time-dependent experiment in MDA-MB-468 cells after treatment with 125 nM XZ9002 at the indicated time points. FIG. 3D: MDA-MB-468 cells were incubated with 125 nM of XZ9002 for 14 h followed by drug washout, and incubation of the cells in drug-free medium for an additional time. Data are presented as representative figures of two independent experiments.

FIG. 4 shows that XZ9002 potently inhibited cell viability in cancer cells. MDA-MB-468, MDA-MB-231, and T47D cells were treated with the indicated compounds at different concentrations for 72 h. Cell viability was assessed with CellTiter-Glo® reagents.

FIG. 5 shows Western blot analysis of HDAC degradation in MDA-MB-468 and T47D cells. The culture of the triple-negative breast cancer cell line MDA-MB-468 and the ER+ breast cancer cell line T47D was exposed to vehicle control (DMSO) and the indicated compounds at specified concentrations for 14 h. Cells were lysed for WB with antibodies against the indicated proteins. Antibodies against acetylated histones at specific sites (H3K27 and H4K5) and acetyl-lysine were used to assess levels of histone acetylation. Levels of HDAC3 were normalized against that of tubulin and % of HDAC3 protein levels in cells treated with the indicated compounds relative to that in DMSO control is shown.

FIG. 6 shows dose response curves of XZ9002 in the NCI-60 cell line panels. The indicated cancer cell lines were treated with XZ9002 at five different doses. The percentage growth relative to the cell numbers before compound exposure was plotted against the XZ9002 concentrations in the log scale.

FIG. 7 shows XZ9002 degrades HDAC3 in vivo. NSG mice bearing MDA-MB-231 xenografts were dosed with vehicle or XZ9002 (50 mg/kg) by i.p. Tumors were dissected from euthanized mice 24 h after dosing. The tumor lysates were analyzed by WB. The quantification is shown in lower panels. ***: p<0.0001 (t-test vs vehicle).

FIGS. 8A-8B show the cellular activity of XZ9002. FIG. 8A shows the cultures of the ER+ cell line MCF7 was exposed to DMSO and the indicated compounds at the specified concentrations for 4 h. Cells were lysed for WB. The relative HDAC3 protein level in each sample is shown. FIG. 8B shows XZ9002 is more potent than XZ9002-NC to inhibit clonogenic growth of BC cells. The indicated BC cell lines were exposed to DMSO, XZ9002, or XZ9002-NC at the indicated concentrations. Colonies were fixed and stained.

FIG. 9 shows the effects of 28c on clonogenic growth of non-small cell lung cancer (NSCLC) cell line H1299. H1299 cells were exposed to DMSO, or 28c at the indicated concentrations. Colonies were fixed and stained after treatment for 10 days.

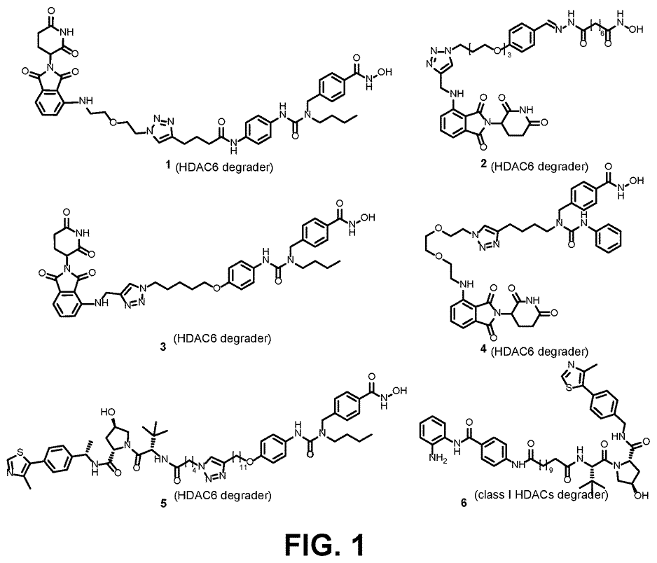

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a peptide,” “a PROTAC,” or “an HDAC3 inhibitor,” include, but are not limited to, mixtures or combinations of two or more such peptides, PROTACs, or HDAC3 inhibitors, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for HDAC3 can be determined in an in vitro or cell-based assay system. Frequently, receptor assays make use of a suitable cell-line, e.g. a cell line that either expresses endogenously a target of interest, or has been transfected with a suitable expression vector that directs expression of a recombinant form of the target. For example, the IC₅₀ for a compound disclosed herein can be determined using mammalian cells transfected with human HDAC3.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C=C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation fora carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O (O)C-A²-C(O)O)ₐ— or -(A¹O (O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Re,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘}₂); —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘}C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂ CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂ NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and =S.

Suitable divalent substituents on a saturated carbon atom of an “optionally substituted” group include the following: ═O, ═S, ═NNR*₂, ═NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

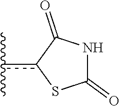

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an a-hydrogen can exist in an equilibrium of the keto form and the enol form.

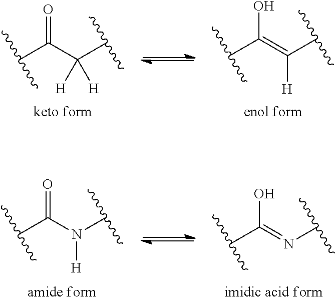 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

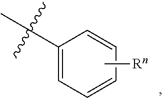

which is understood to be equivalent to a formula:

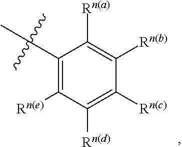

wherein n is typically an integer. That is, Rⁿ is understood to represent five independent substituents, R^{n(a)}, R^{n(b)}, R^{n(c)}, R^{n(d)}, , and R^{n(e)}. By “independent substituents,” it is meant that each R substituent can be independently defined. For example, if in one instance R^{n(a)} is halogen, then R^{n(b)} is not necessarily halogen in that instance.

The squiggle line placed on the bonds in the structures provided herein represents a bond to another group. For example, in the structure below which represents X in formula I:

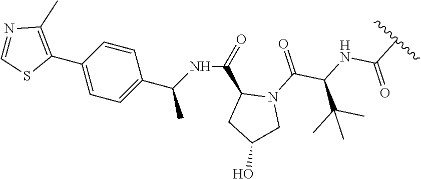 [SMILES: *C(=O)N[C@H](C(=O)N1C[C@H](O)C[C@H]1C(=O)N[C@@H](C)c1ccc(-c2scnc2C)cc1)C(C)(C)C]

the squiggle line through the bond indicates that another group (e.g., L₁) is bonded to the structure above.

The term “omitted” when referenced in the structures described herein means a group is not present but a bond is present. For example, in the structure

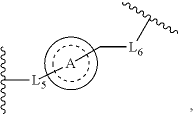

when L₆ is omitted, the structure can be represented as follows:

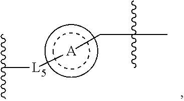

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the term “effective amount” refers to an amount that is sufficient to achieve the desired modification of a physical property of the composition or material. For example, an “effective amount” of an HDAC3 inhibitor refers to an amount that is sufficient to achieve the desired improvement in the property modulated by the formulation component, e.g. achieving the desired level of inhibition of HDAC3 activity, or, in the case of the PROTACs disclosed herein, achieving the desired level of degradation of HDAC3. The specific level in terms of wt % in a composition required as an effective amount will depend upon a variety of factors including the amount and type of compound, levels of other HDAC enzymes present in the cell, type of cell or tissue, and type of cancer or other disorder that is to be treated..

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Methods of Making and Using the Compounds

In one aspect, disclosed herein are novel PROTACs that can efficiently degrade HDAC3. In another aspect, the compounds induce HDAC3 degradation in a dose- and time-dependent manner. At a high concentrations, these novel PROTACs also degrade HDAC1 and HDAC2. In still another aspect, MOA studies have validated that PROTAC-induced degradation is mediated by both E3 ligase and the ubiquitin/proteasome system (UPS). In a further aspect, cell viability assay revealed that HDAC3 degrader XZ9002 has potent antiproliferative activity against cancer cells. In one aspect, due to the catalytic mechanism of action and improved isoform selectivity, this class of novel HDAC3 degraders may overcome the dose-limiting toxicity associated with conventional HDACi. In a further aspect, this may be crucial to fulfilling the considerable therapeutic potential of HDAC inhibition and/or degradation in the clinic. Considering the complicated functions of differentiate HDAC isoforms, in one aspect, the different compounds disclosed herein can be part of a useful toolkit to chemically dissect the functions of HDAC family proteins.

In one aspect, disclosed herein is a compound having a structure represented by Formula I:

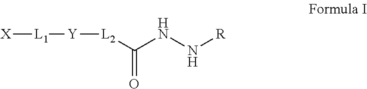

wherein X can be an E3 ligase targeting moiety;

L₁ can be a C2-C12 alkyl group;

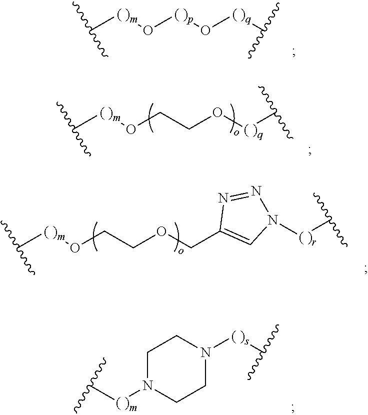

or a combination thereof;
m is from 1 to 11;
is from 0 to 10;
p is from 2 to 4;
q is from 1 to 4;
r is from 0 to 10; and
s is from 1 to 10;

wherein Y can be

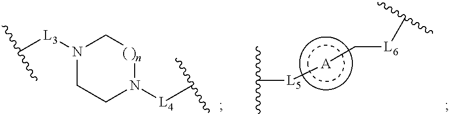

or a combination thereof;
wherein L₃ is omitted or can be a keto group, an amide group, a sulfonyl group, or
a combination thereof;
L₄ is omitted or can be a keto group, a sulfonyl group, a C1-C2 alkyl group, —C(O)CH₂—, —CH═CH—, or a combination thereof;
n is from 1 to 3;
A can be a substituted or unsubstituted monocyclic aryl group, a substituted or unsubstituted monocyclic heteroaryl group, or a combination thereof;
L₅ is omitted or can be an amide group, a sulfonamide group, a keto group, oxygen, —CH═CH—, —CH₂C(O)—NH—, or a combination thereof;
L₆ is omitted or can be oxygen, a keto group, an amide group, a sulfonamide group, or a combination thereof;

wherein L₂ can be a monocyclic aryl group, monocyclic heteroaryl group, or a combination thereof;

and wherein R can be a substituted or unsubstituted C₁-C6 linear or branched alkyl group, a C3-C6 substituted or unsubstituted cycloalkyl group, or a combination thereof.

In another aspect, the E3 ligase targeting moiety can be

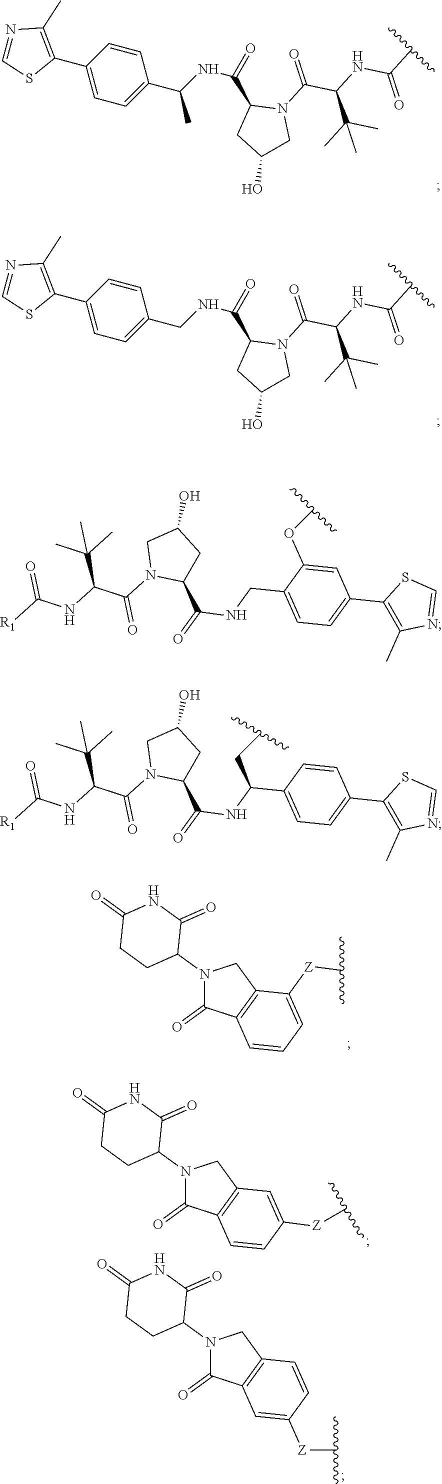

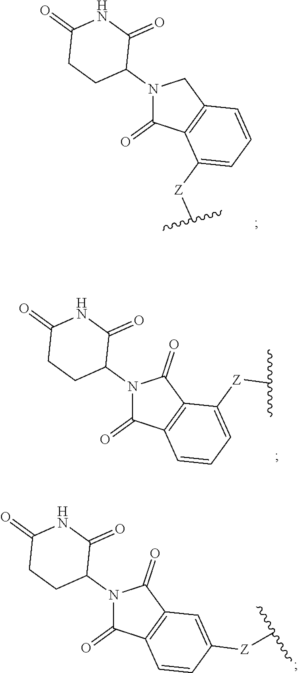

or a combination thereof;

wherein R₁ can be methyl,

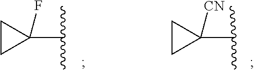 [SMILES: *C1(F)CC1 ; *C1(C#N)CC1]

or a combination thereof; and

wherein Z can be oxygen, NH, methylene, or a combination thereof.

In another aspect, L₁ in the disclosed compounds can be a C2-C8 alkyl group;

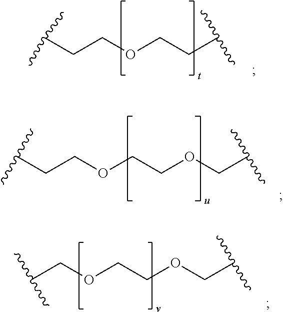

or a combination thereof, wherein t, u, and v are, independently, from 0 to 6. In another aspect, L₁ can be a C2, C4, C6, or C8 alkyl group.

In one aspect, L₁ can be

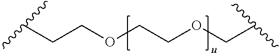

and u can be 1, 2, or 3. In another aspect, L₁ can be

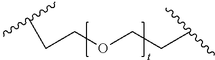

and t can be 1, 2, or 3.

In one aspect, Y can be

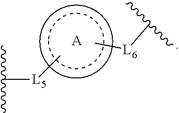

Further in this aspect, L₅ can be an amide group and L₆ can, in some aspects, be omitted.

In another aspect, L₂ can be a monocyclic aryl group such as, for example,

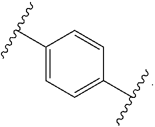 [SMILES: *c1ccc(*)cc1]

In another aspect, R can be a substituted or unsubstituted C1-C6 linear or branched alkyl group, a C3-C6 substituted or unsubstituted cycloalkyl group; or a combination thereof. In one aspect, R is

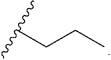 [SMILES: *CCC]

In another aspect, non-limiting examples of R can include, but are not limited to,

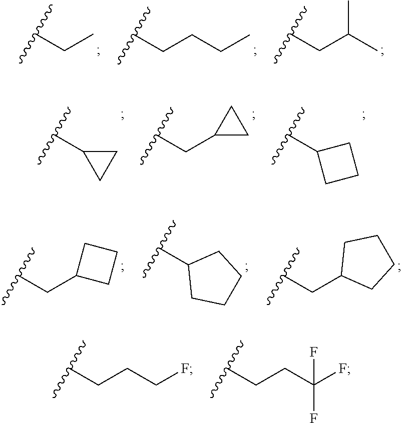

and combinations thereof.

In one aspect, the compound disclosed herein can have one of the following formulas:

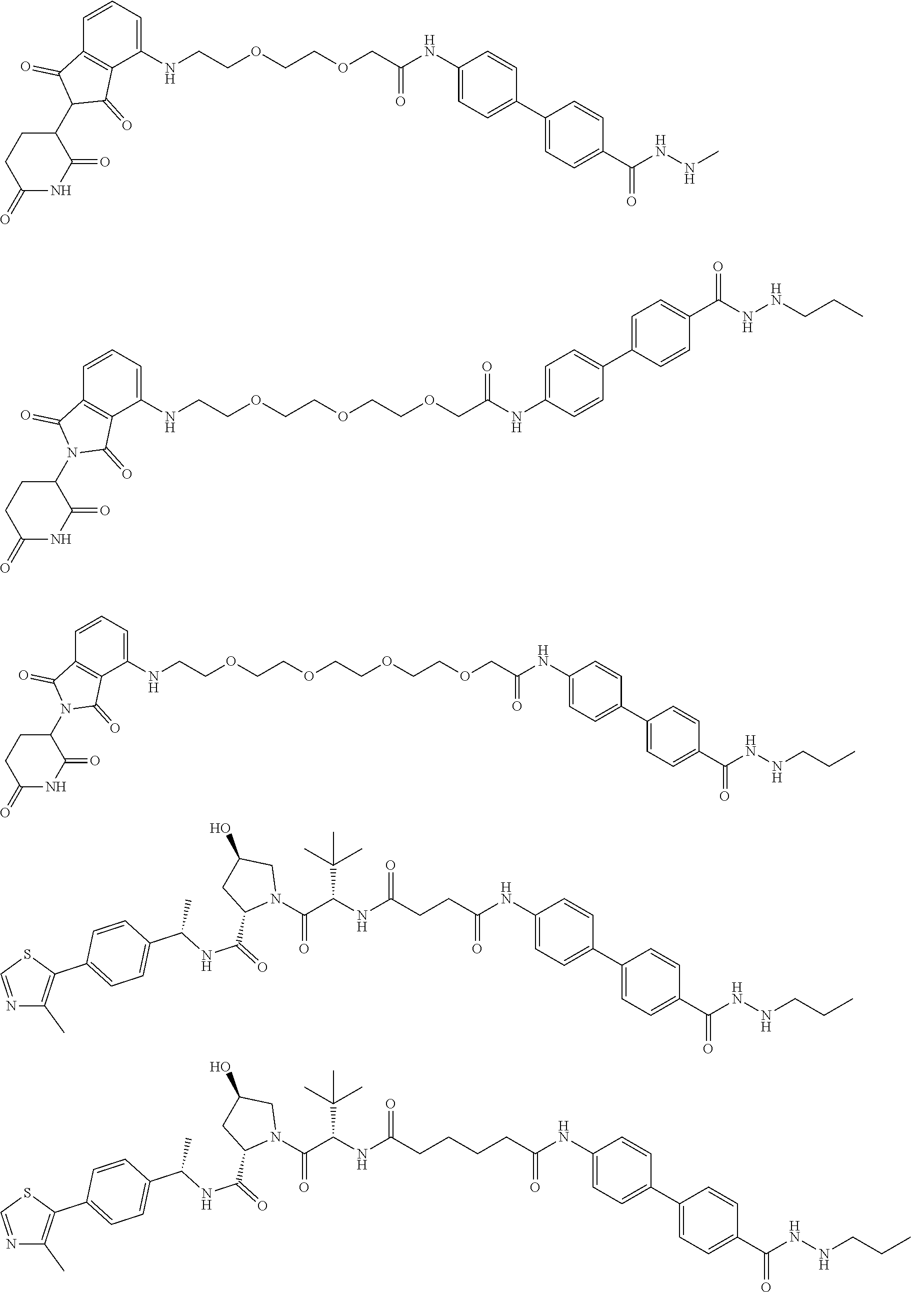 [SMILES: CNNC(=O)c1ccc(-c2ccc(NC(=O)COCCOCCNc3cccc4c3C(=O)C(C3CCC(=O)NC3=O)C4=O)cc2)cc1 ; CCCNNC(=O)c1ccc(-c2ccc(NC(=O)COCCOCCOCCNc3cccc4c3C(=O)N(C3CCC(=O)NC3=O)C4=O)cc2)cc1 ; CCCNNC(=O)c1ccc(-c2ccc(NC(=O)COCCOCCOCCOCCNc3cccc4c3C(=O)N(C3CCC(=O)NC3=O)C4=O)cc2)cc1 ; CCCNNC(=O)c1ccc(-c2ccc(NC(=O)CCC(=O)N[C@H](C(=O)N3C[C@H](O)C[C@H]3C(=O)N[C@@H](C)c3ccc(-c4scnc4C)cc3)C(C)(C)C)cc2)cc1 ; CCCNNC(=O)c1ccc(-c2ccc(NC(=O)CCCCC(=O)N[C@H](C(=O)N3C[C@H](O)C[C@H]3C(=O)N[C@@H](C)c3ccc(-c4scnc4C)cc3)C(C)(C)C)cc2)cc1]

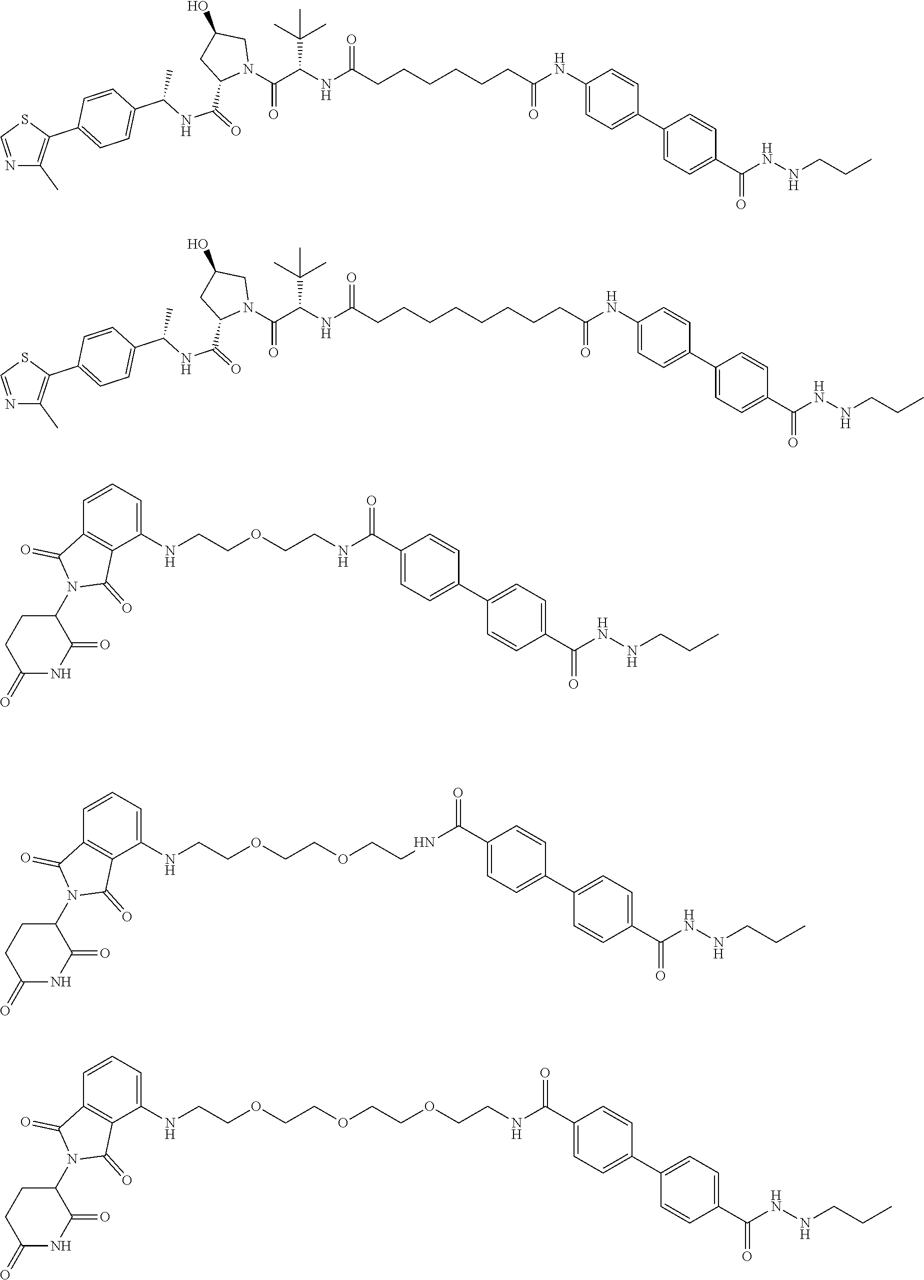 [SMILES: CCCNNC(=O)c1ccc(-c2ccc(NC(=O)CCCCCCC(=O)N[C@H](C(=O)N3C[C@H](O)C[C@H]3C(=O)N[C@@H](C)c3ccc(-c4scnc4C)cc3)C(C)(C)C)cc2)cc1 ; CCCNNC(=O)c1ccc(-c2ccc(NC(=O)CCCCCCCCC(=O)N[C@H](C(=O)N3C[C@H](O)C[C@H]3C(=O)N[C@@H](C)c3ccc(-c4scnc4C)cc3)C(C)(C)C)cc2)cc1 ; CCCNNC(=O)c1ccc(-c2ccc(C(=O)NCCOCCNc3cccc4c3C(=O)N(C3CCC(=O)NC3=O)C4=O)cc2)cc1 ; CCCNNC(=O)c1ccc(-c2ccc(C(=O)NCCOCCOCCNc3cccc4c3C(=O)N(C3CCC(=O)NC3=O)C4=O)cc2)cc1 ; CCCNNC(=O)c1ccc(-c2ccc(C(=O)NCCOCCOCCOCCNc3cccc4c3C(=O)N(C3CCC(=O)NC3=O)C4=O)cc2)cc1]

In one aspect, disclosed herein is a method for synthesizing a compound of formula I, the method including the steps of:
a) (i) reacting a compound having formula II with an aldehyde in a first solvent to produce and (ii) adding a reducing agent in a second solvent to produce a compound of formula III;

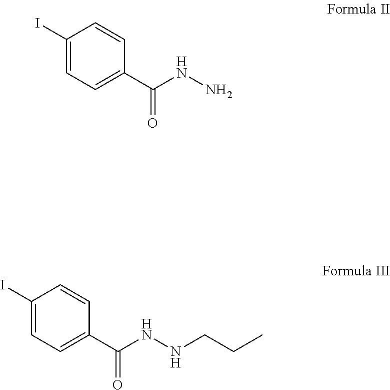

b) reacting the compound of formula III with a protecting group source and a first base in a third solvent to produce a compound of formula IV;

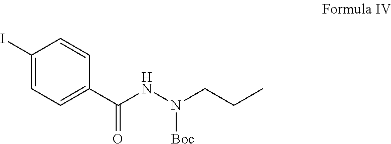 [SMILES: CCCN(NC(=O)c1ccc(I)cc1)C(=O)OC(C)(C)C]

c) reacting the compound of formula IV with a substituted aromatic compound, a first catalyst, and a second base in a fourth solvent at a first temperature to produce a compound of formula V;

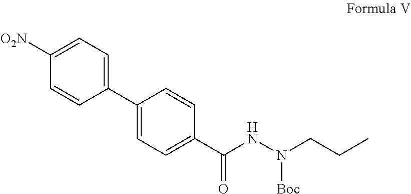 [SMILES: CCCN(NC(=O)c1ccc(-c2ccc([N+](=O)[O-])cc2)cc1)C(=O)OC(C)(C)C]

d) reacting the compound of formula V with a second catalyst and a hydrogenation agent in a fifth solvent to produce a compound of formula VI;

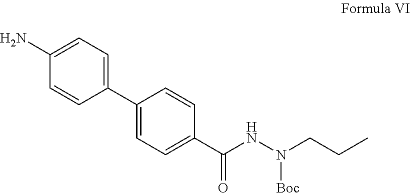 [SMILES: CCCN(NC(=O)c1ccc(-c2ccc(N)cc2)cc1)C(=O)OC(C)(C)C]

e) (i) reacting the compound of formula VI with an anhydride and a third base in a sixth solvent and (ii) followed by addition of a first acid to produce a compound of formula VII;

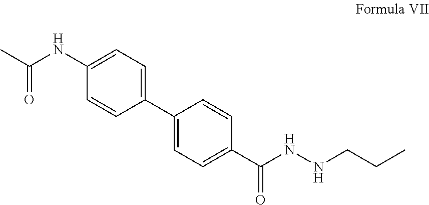 [SMILES: CCCNNC(=O)c1ccc(-c2ccc(NC(C)=O)cc2)cc1]

f) (i) reacting the compound of formula VII with an E3 ligase-targeting moiety of formula VIII or IX, a coupling agent, and a fourth base in a seventh solvent, (ii) followed by addition of a second acid to produce the compound of formula I;

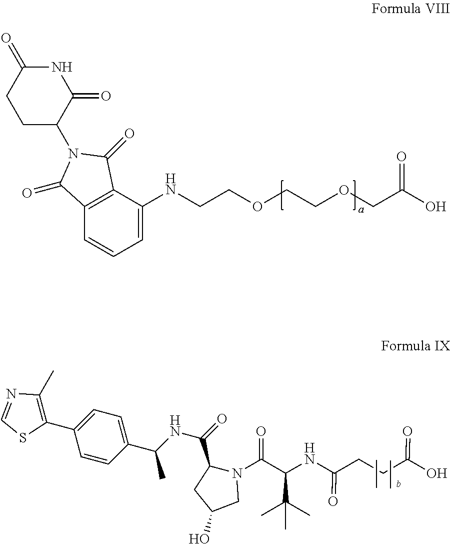

g) wherein a is from 1 to 3 and b is from 1 to 7.

In one aspect, the aldehyde can be propionaldehyde. In a further aspect, the first solvent can be methanol, ethanol, isopropanol, dichloromethane, tetrahydrofuran, 1,4-dioxane, or a combination thereof. In another aspect, the reducing agent can be sodium borohydride, sodium triacetoxyborohydride, sodium cyanoborohydride, or a combination thereof. In any of these aspects, the second solvent can be methanol, ethanol, isopropanol, methylene chloride, tetrahydrofuran, 1,4-dioxane, or a combination thereof. In one aspect, the protecting group source can be di-tert-butyl-dicarbonate. In one aspect, the first base can be trimethylamine, N,N-diisopropylethylamine, N-methylmorpholine, pyridine, 2,6-lutidine, or a combination thereof.

In one aspect, the third solvent can be dichloromethane, tetrahydrofuran, 1,4-dioxane, or a combination thereof, and the substituted aromatic compound can be 4-nitrophenylboronic acid. In one aspect, the first catalyst can be Pd(PPh₃)₄. In another aspect, the second base can be sodium carbonate, potassium carbonate, cesium carbonate, trimethylamine, N,N-diisopropylethylamine, N-methylmorpholine, pyridine, 2,6-lutidine, or a combination thereof. In still another aspect, the fourth solvent can be toluene, ethanol, water, tetrahydrofuran, 1,4-dioxane, dimethylformamide, or a combination thereof. In one aspect, the first temperature is from about 60 to about 120° C., or is about 60, 65, 70, 75, 80, 85, 90, 95, 100, 105, 110, 115, or about 120° C., or a combination of any of the foregoing values, or a range encompassing any of the foregoing values. In one aspect, the first temperature is about 90° C.

In another aspect, the second catalyst can be Pd/C and the hydrogenation agent can be H₂. In one aspect, the fifth solvent can be ethyl acetate methanol, ethanol, isopropanol, tetrahydrofuran, 1,4-dioxane, or a combination thereof. In one aspect, the E3-targeting moiety can have Formula VIII and a can be 1, 2, or 3. In another aspect, the E3-targeting moiety can be Formula IX and b can be 1, 3, 5, or 7. In one aspect, the anhydride can be acetic anhydride. In another aspect, the third base can be trimethylamine, N,N-diisopropylethylamine, N-methylmorpholine, pyridine, 2,6-lutidine, or a combination thereof. In one aspect, the sixth solvent can be dichloromethane, tetrahydrofuran, 1,4-dioxane, dimethylformamide, or a combination thereof. In one aspect, the first acid can be trifluoroacetic acid, methanesulfonic acid, p-toluenesulfonic acid, hydrochloric acid, or a combination thereof. In another aspect, the coupling agent can be hexafluorophosphate azabenzotriazole tetramethyl uranium (HATU), 1-ethyl-3-(3-dimethylaminopropyl)carbodiimide, N,N′-dicyclohexylcarbodiimide, propanephosphonic acid anhydride, benzotriazole-1-yl-oxy-tris-pyrrolidino-phosphonium hexafluorophosphate. In one aspect, the fourth base can be trimethylamine, N,N-diisopropylethylamine, N-methylmorpholine, pyridine, 2,6-lutidine, or a combination thereof, and the seventh solvent can be dichloromethane, tetrahydrofuran, 1,4-dioxane, dimethylformamide, or a combination thereof. In one aspect, the second acid can be trifluoroacetic acid, methanesulfonic acid, p-toluenesulfonic acid, hydrochloric acid, or a combination thereof.

In another aspect, disclosed is a method for synthesizing a compound of Formula I, the method including the following steps:
a) (i) reacting a compound having formula II with an aldehyde in a first solvent to produce and (ii) adding a reducing agent in a second solvent to produce a compound of formula III;

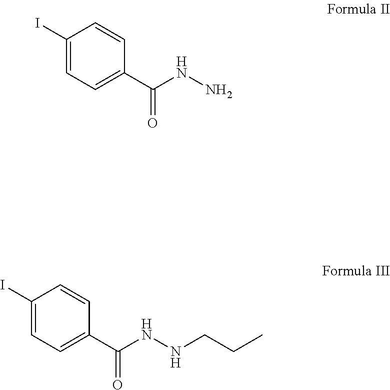

b) reacting the compound of formula III with a protecting group source and a first base in a third solvent to produce a compound of formula IV;

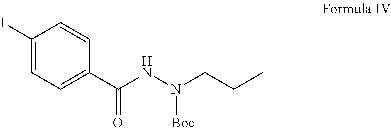 [SMILES: CCCN(NC(=O)c1ccc(I)cc1)C(=O)OC(C)(C)C]

c) reacting the compound of formula IV with an aromatic acid, a first catalyst, and a second base in a third solvent at a first temperature to produce a compound of formula X;

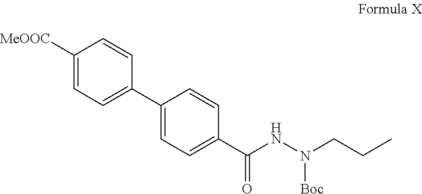 [SMILES: CCCN(NC(=O)c1ccc(-c2ccc(C(=O)OC)cc2)cc1)C(=O)OC(C)(C)C]

d) reacting the compound of formula X with a third base in a fourth solvent at a second temperature to produce a compound of formula XI;

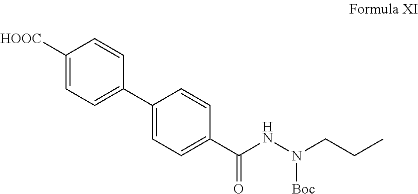 [SMILES: CCCN(NC(=O)c1ccc(-c2ccc(C(=O)O)cc2)cc1)C(=O)OC(C)(C)C]

e) reacting the compound of formula XI with an E3 ligase-targeting moiety of formula XII with a fourth base and a coupling agent in a fifth solvent followed by addition of an acid to produce the compound of formula I

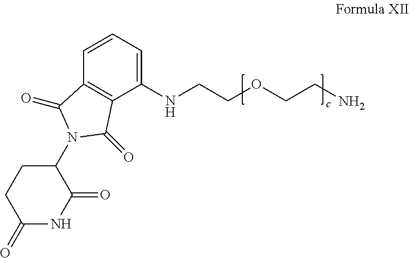

wherein c is from 1 to 3.

In one aspect, the aldehyde can be propionaldehyde and the first solvent can be methanol, ethanol, isopropanol, dichloromethane, tetrahydrofuran, 1,4-dioxane, or a combination thereof. In another aspect, the reducing agent can be sodium borohydride, sodium triacetoxyborohydride, sodium cyanoborohydride, or a combination thereof. In any of these aspects, the second solvent can be methanol, ethanol, isopropanol, methylene chloride, tetrahydrofuran, 1,4-dioxane, or a combination thereof. In one aspect, the protecting group source can be di-tert-butyl-dicarbonate. In one aspect, the first base is triethylamine, N,N-diisopropylethylamine, N-methylmorpholine, pyridine, 2,6-lutidine, or a combination thereof. In another aspect, the third solvent can be dichloromethane, tetrahydrofuran, 1,4-dioxane, or a combination thereof.

In one aspect, the aromatic acid can be (4-(methoxycarbonyl)phenyl)boronic acid. In one aspect, the first catalyst can be Pd(PPh₃)₄. In one aspect, the second base can be sodium carbonate, potassium carbonate, cesium carbonate, trimethylamine, N,N-diisopropylethylamine, N-methylmorpholine, pyridine, 2,6-lutidine, or a combination thereof. In another aspect, the third solvent can be toluene, ethanol, water, tetrahydrofuran, 1,4-dioxane, dimethylformamide, or a combination thereof. In one aspect, the first temperature is from about 60 to about 120° C., or is about 60, 65, 70, 76, 80, 85, 90, 95, 100, 105, 110, 115, or about 120° C., or a combination of any of the foregoing values, or a range encompassing any of the foregoing values. In another aspect, the third base can be lithium hydroxide, sodium hydroxide, potassium hydroxide, potassium carbonate, or a combination thereof.

In one aspect, the fourth solvent can be methanol, ethanol, isopropanol, tetrahydrofuran, 1,4-dioxane, water, or a combination thereof, and the second temperature can be about 25 to about 80° C., or can be about 25, 30, 35, 40, 45, 50, 55, 60, 65, 70, 75, or about 80° C., or a combination of any of the foregoing values, or a range encompassing any of the foregoing values. In one aspect, c can be 1, 2, or 3. In a further aspect, the fourth base can be trimethylamine, N,N-diisopropylethylamine, N-methylmorpholine, pyridine, 2,6-lutidine, or a combination thereof. In one aspect, the coupling agent can be hexafluorophosphate azobenzotriazole tetramethyo uronium (HATU), 1-ethyl-3-(3-dimethylaminopropyl)carbodiimide, N,N′-dicyclohexylcarbodiimide, propanephosphonic acid anhydride, benzotriazole-1-yl-oxy-tris-pyrrolidino-phosphonium hexafluorophosphate, or a combination thereof. In one aspect, the fifth solvent can be dichloromethane, tetrahydrofuran, 1,4-dioxane, dimethylformamide, or a combination thereof. In another aspect, the acid can be trifluoroacetic acid, methanesulfonic acid, p-toluenesulfonic acid, hydrochloric acid, or a combination thereof.

In another aspect, a method for synthesizing a compound of Formula I comprises:

reacting the compound of formula X with an HDAC-targeting moiety of formula IX to produce the compound of formula I;

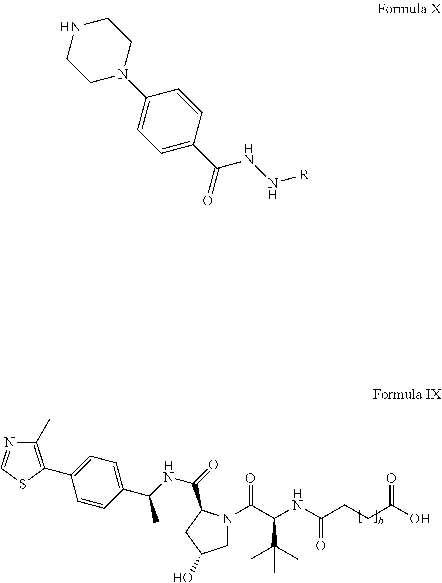

wherein b is from 1 to 10, and
R comprises a substituted or unsubstituted C1-C6 linear or branched alkyl group, a C3-C6 substituted or unsubstituted cycloalkyl group, or a combination thereof.

In another aspect, a method for synthesizing a compound of Formula I comprises:

reacting the compound of formula X with an HDAC-targeting moiety of formula IX to produce the compound of formula I;

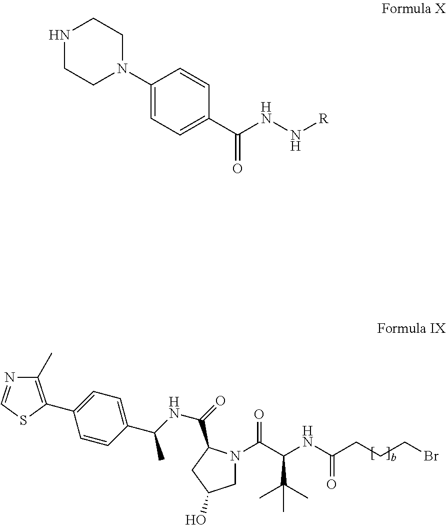

wherein b is from 1 to 10, and

R comprises a substituted or unsubstituted C1-C6 linear or branched alkyl group, a C3-C6 substituted or unsubstituted cycloalkyl group, or a combination thereof.

Non-limiting procedures for making and purifying the compounds described herein are provided in the Examples.

### Therapeutic Agents

As used herein, “histone deacetylase” and “HDAC” can be used interchangeably. In another aspect, “HDAC3” refers to an enzyme encoded by a gene in humans with a cytogenetic location of 5q31.3 and a molecular location of base pairs 141,620,875 to 141,636,855 on chromosome 5 (Homo sapiens Annotation Release 109, GRCh38.p12). The gene structure in humans includes 15 exons. HDAC3 has an EC classification of 3.5.1.98; an intracellular location within the nucleus and cytoplasm; and catalyzes the deacetylation of lysine residues on the N-terminal part of the core histones (H2A, H2B, H3, and H4) and several non-histone substrates, which may be important for epigenetic repression, transcriptional regulation, cell cycle progression, and other developmental events. HDAC has also been referred to as HD3, RPD3-2, and SMAP45. In one aspect, low levels of acetylation induced with HDAC activity may be associated with gene silencing. Further in this aspect, reducing aberrant HDAC activity may allow the expression of genes silenced by this mechanism. In one aspect, disclosed herein are selective inhibitors and/or PROTACs for HDAC3.

As used herein, “E3 ligase” (also known as “ubiquitin ligase” and “E3 ubiquitin ligase”) is a protein that recruits an E2 ubiquitin-conjugating enzyme that is loaded with ubiquitin. The E3 ligase recognizes a protein substrate and assists or directly catalyzes the transfer of ubiquitin from the E2 to the substrate. Once conjugated to ubiquitin, the protein substrate is targeted for destruction by the proteasome.

As used herein, “CRBN” is a gene that encodes the protein cereblon. Cereblon is involved in various activities including, but not limited to, gene expression and assembly of other proteins related to cell proliferation and metabolism. Cereblon further assist certain drugs in performing their immunomodulatory and anti-tumor effects. In one aspect, the compounds disclosed herein recruit CRBN as E3 ubiquitin ligase to induce proteasomal degradation.

As used herein, “VHL” is a tumor suppressor gene encoding the Von Hippel-Lindau protein, which has roles in functions ranging from cytokine signaling, regulation of senescence, oxygen sensing, and microtubule stability. In one aspect, the compounds disclosed herein may recruit VHL as E3 ubiquitin ligase to induce proteasomal degradation.

As used herein, a “PROTAC” is a proteolysis targeting chimera, or a small molecule having two active domains and a linker, wherein the PROTAC is capable of degrading or inactivating unwanted proteins. In a further aspect, as a mechanism of action, a PROTAC activates intracellular proteolysis. In one aspect, one of the active domains engages an E3 ubiquitin ligase and the other binds the target protein (e.g., HDAC3). Disclosed herein are PROTACs useful in recruiting E3 ligases (e.g. CRBN, VHL) to assist in the degradation of HDAC3.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain. In one aspect, “treating” and “treatment” includes an improved pharmacological and/or physiological effect when administered a compound described herein when compared to not administering the compound (i.e., the control).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Described herein are HDAC3 inhibitors and/or PROTACs that have therapeutic or clinical utility. Also described herein are methods of synthesizing the HDAC3 inhibitors and PROTACs. Also described herein are methods of administering the HDAC3 inhibitors and PROTACs to a subject in need thereof. In some aspects, the subject can have cancer. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compounds

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thomber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require inhibition or degradation of HDAC3 activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating HDAC3 activity (e.g., treatment of one or more cancers or other disorders associated with HDAC3 dysfunction) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an HDAC3 inhibitor or PROTAC. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional chemotherapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

In a further aspect, the present disclosure provides methods of treatment comprising administration of a therapeutically effective amount of a disclosed compound or pharmaceutical composition as disclosed herein above to a subject in need thereof.

In one aspect, disclosed herein is a pharmaceutical composition including a therapeutically effective amount of a compound disclosed herein or a pharmaceutically acceptable salt, solvate, or polymorph thereof, and a pharmaceutically acceptable carrier.

### Methods for Treatment of Disorders in Mammals

In one aspect, disclosed herein is a method for the treatment of a disorder in a mammal, the method including the step of administering to the mammal a therapeutically effective amount of at least one disclosed compound, or a pharmaceutically acceptable salt thereof, or the disclosed pharmaceutical composition. In some aspects, the mammal is a human. In another aspect, the mammal has been diagnosed with a need for treatment of the disorder prior to the administering step. In some aspects, the method further includes the step of identifying a mammal in need of treatment of the disorder. In one aspect, the disorder is selected from breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof.

In another aspect, disclosed herein is a method for inhibiting the activity of at least one histone deacetylase enzyme in a mammal, including the step of administering to the mammal a therapeutically effective amount of at least one disclosed compound, or a pharmaceutically acceptable salt thereof, or a disclosed pharmaceutical composition. In one aspect, the mammal is a human. In another aspect, the histone deacetylase enzyme is histone deacetylase 3 (HDAC3). In still another aspect, the compound exhibits an IC₅₀ of less than about 0.55 μM for HDAC3, or of less than 0.5, 0.45, 0.4, 0.35, 0.3, 0.25, 0.2, 0.15, or less than about 0.1 μM for HDAC3, or a combination of any of the foregoing values, or a range encompassing any of the foregoing values. In another aspect, the compound exhibits a lower IC₅₀ for HDAC3 than for HDAC2 or than for HDAC1. In one aspect, the IC₅₀ for HDAC2 is from about 4 to about 10 times higher than the IC₅₀ for HDAC3, or is about 4, 5, 6, 7, 8, 9, or about 10 times higher, or a combination of any of the foregoing values, or a range encompassing any of the foregoing values. In another aspect, the IC₅₀ for HDAC1 is from about 1.8 to about 3 times the IC₅₀ for HDAC3, or is about 1.8, 2, 2.25, 2.5, 2.75, or about 3 times the IC₅₀ for HDAC3, or a combination of any of the foregoing values, or a range encompassing any of the foregoing values. In a further aspect, the mammal has been diagnosed with a need for inhibiting the activity of at least one histone deacetylase enzyme prior to the administering step. In another aspect, the method further includes identifying a mammal with a need for inhibiting the activity of at least one histone deacetylase enzyme prior to the administering step.

### Kits

In a further aspect, the present disclosure relates to kits comprising at least one disclosed compound, or a pharmaceutically acceptable salt, hydrate, solvate, or polymorph thereof, and one or more of: (a) at least one agent known to decrease HDAC3 activity; (b) at least one agent known to treat a disorder associated with aberrant HDAC3 activity; (c) instructions for treating a disorder associated with aberrant HDAC3 activity; or (d) instructions for administering the compound in connection with another cancer therapy.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

In one aspect, disclosed herein is a kit containing at least one disclosed compound or a pharmaceutically acceptable salt thereof and one or more of (a) at least one agent known to increase the activity of at least one histone deacetylase enzyme, (b) at least one agent known to decrease the activity of at least one histone deacetylase enzyme, and (c) at least one agent known to treat breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof. In some aspects, the kit further includes instructions for treating breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease.

In one aspect, the disclosed compound and the at least one agent are co-formulated and/or co-packaged.

### Research Tools

The disclosed compounds and pharmaceutical compositions have activity as inhibitors of HDAC3 and/or as compounds that target HDAC3 by binding and, subsequently, by recruiting proteolytic enzymes to degrade HDAC3. As such, the disclosed compounds are also useful as research tools. Accordingly, one aspect of the present disclosure relates to a method of using a compound of the invention as a research tool, the method comprising conducting a biological assay using a compound of the invention. Compounds of the invention can also be used to evaluate new chemical compounds. Thus another aspect of the invention relates to a method of evaluating a test compound in a biological assay, comprising: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include an IC₅₀ assay that can be conducted in vitro or in a cell culture system. Still another aspect of the invention relates to a method of studying a biological system, e.g., a model animal for a clinical condition, or biological sample comprising an HDAC3 protein, the method comprising: (a) contacting the biological system or sample with a compound of the invention; and (b) determining the effects caused by the compound on the biological system or sample.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered Aspects, which should not be confused with the claims.

Aspect 1. A compound having a structure represented by Formula I or a pharmaceutically acceptable salt, solvate, or polymorph thereof:

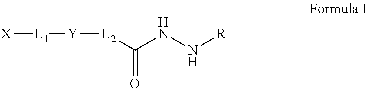

wherein X comprises an E3 ligase targeting moiety;
wherein L₁ comprises a C2-C12 alkyl group;

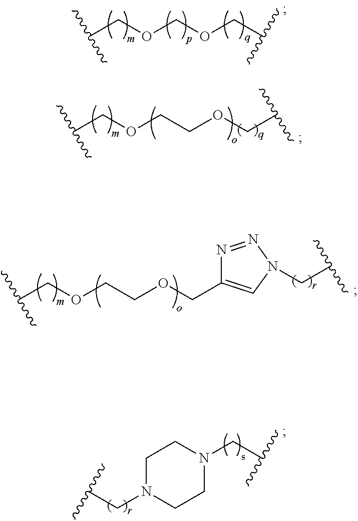

or a combination thereof;
   wherein m is from 1 to 11;
   is from 0 to 10;
   p is from 2 to 4;
   q is from 1 to 4;
   r is from 0 to 10; and
   s is from 1 to 10;
wherein Y comprises

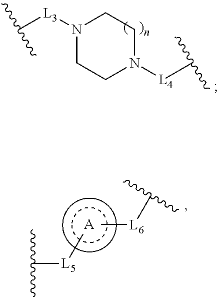

or a combination thereof;
   wherein L₃ is omitted or comprises a keto group, an amide group, a sulfonyl group, or a combination thereof;
   L₄ is omitted or comprises a keto group, a sulfonyl group, a C1-C2 alkyl group, —C(O)CH₂—, —CH═CH—, or a combination thereof;
   n is from 1 to 3;
   A comprises a substituted or unsubstituted monocyclic aryl group, a substituted or unsubstituted monocyclic heteroaryl group; or a combination thereof;
   L₅ is omitted or comprises an amide group, a sulfonamide group; a keto group; oxygen; —CH═CH—; —CH₂C(O)—NH—; or a combination thereof; and
   L₆ is omitted or comprises oxygen, a keto group, an amide group, a sulfonamide group, or a combination thereof;
wherein L₂ comprises a monocyclic aryl group, monocyclic heteroaryl group, or a combination thereof;
and wherein R comprises a substituted or unsubstituted C1-C6 linear or branched alkyl group, a C3-C6 substituted or unsubstituted cycloalkyl group, or a combination thereof.
   Aspect 2. The compound of aspect 1, wherein the E3 ligase targeting moiety comprises

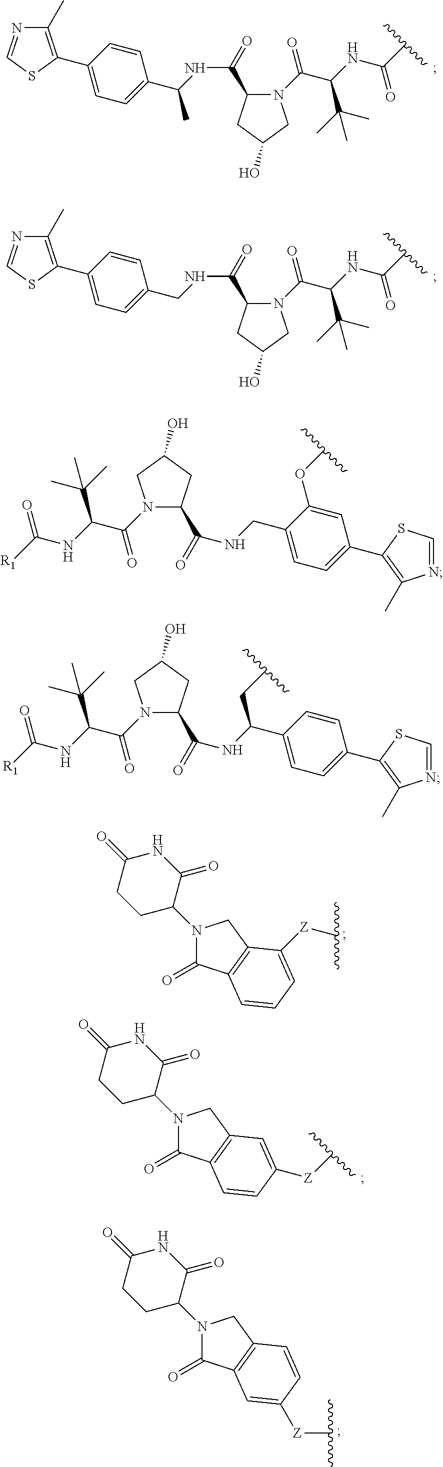

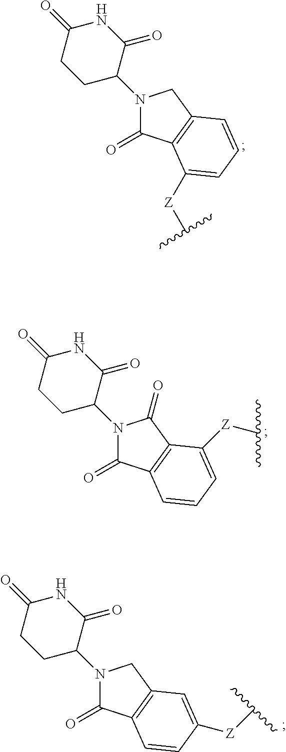

or a combination thereof;
wherein R₁ comprises methyl,

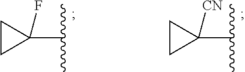 [SMILES: *C1(F)CC1 ; *C1(C#N)CC1]

or a combination thereof; and
wherein Z comprises oxygen, NH, methylene, or a combination thereof.
   Aspect 3. The compound of aspect 1 or 2, wherein the E3 ligase targeting moiety comprises

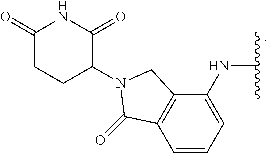 [SMILES: *Nc1cccc2c1CN(C1CCC(=O)NC1=O)C2=O]

Aspect 4. The compound of aspect 1 or 2, wherein the E3 ligase targeting moiety comprises

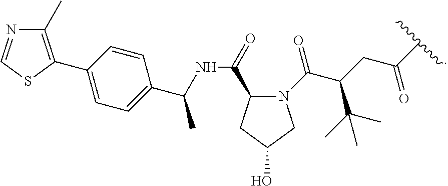 [SMILES: *C(=O)C[C@H](C(=O)N1C[C@H](O)C[C@H]1C(=O)N[C@@H](C)c1ccc(-c2scnc2C)cc1)C(C)(C)C]

Aspect 5. The compound of any of aspects 1-4, wherein L¬1 comprises a C2-C8 alkyl group;

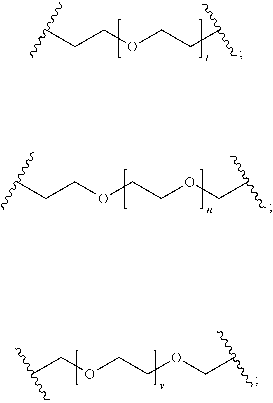

or a combination thereof; and
wherein t, u, and v are independently from 0 to 6.
   Aspect 6. The compound of aspect 5, wherein L₁ comprises a C2 alkyl group.
   Aspect 7. The compound of aspect 5, wherein L₁ comprises a C4 alkyl group.
   Aspect 8. The compound of aspect 5, wherein L₁ comprises a C6 alkyl group.
   Aspect 9. The compound of any of aspects 1-4, wherein L₁ comprises a C8 alkyl group.
   Aspect 10. The compound of aspect 5, wherein L₁ comprises

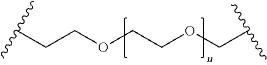

and u is 1.
Aspect 11. The compound of aspect 5, wherein L₁ comprises

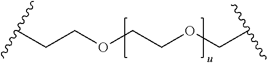

and u is 2.

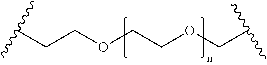

Aspect 12. The compound of aspect 5, wherein L₁ comprises

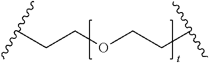

and u is 3.
Aspect 13. The compound of aspect 5, wherein L₁ comprises

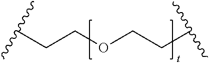

and t is 1.
Aspect 14. The compound of aspect 5, wherein L₁ comprises

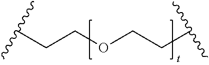

and t is 2.
Aspect 15. The compound of aspect 5, wherein L₁ comprises

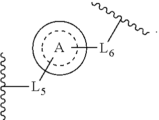

and t is 3.
Aspect 16. The compound of any of aspects 1-15, wherein Y comprises
Aspect 17. The compound of aspect 16, wherein L₅ comprises an amide group.
Aspect 18. The compound of aspect 16 or 17, wherein L₆ is omitted.
Aspect 19. The compound of any of aspects 1-18, wherein L₂ comprises a monocyclic aryl group.
Aspect 20. The compound of aspect 19, wherein L₂ comprises

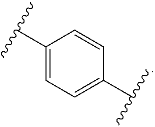 [SMILES: *c1ccc(*)cc1]

Aspect 21. The compound of any of aspects 1-20, wherein R comprises a substituted or unsubstituted C1-C6 linear or branched alkyl group, a C3-C6 substituted or unsubstituted cycloalkyl group, or a combination thereof.
Aspect 22. The compound of aspect 21, wherein R comprises

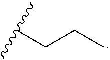 [SMILES: *CCC]

Aspect 23. The compound of aspect 1, having a structure represented by a formula:

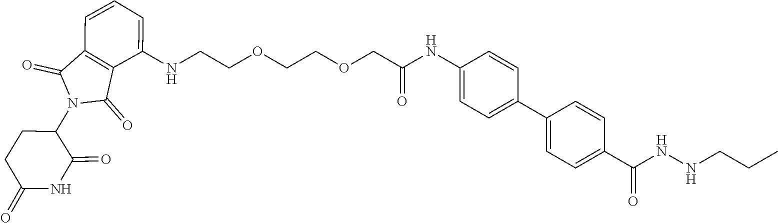 [SMILES: CCCNNC(=O)c1ccc(-c2ccc(NC(=O)COCCOCCNc3cccc4c3C(=O)N(C3CCC(=O)NC3=O)C4=O)cc2)cc1]

Aspect 24. The compound of aspect 1, having a structure represented by a formula:

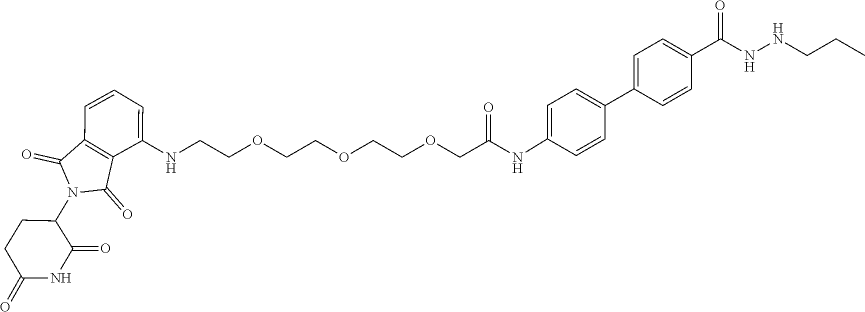 [SMILES: CCCNNC(=O)c1ccc(-c2ccc(NC(=O)COCCOCCOCCNc3cccc4c3C(=O)N(C3CCC(=O)NC3=O)C4=O)cc2)cc1]

Aspect 25. The compound of aspect 1, having a structure represented by a formula:

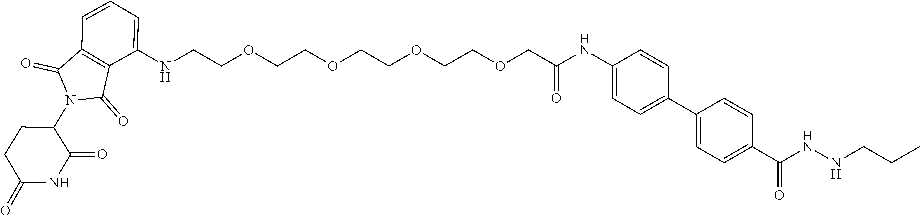 [SMILES: CCCNNC(=O)c1ccc(-c2ccc(NC(=O)COCCOCCOCCOCCNc3cccc4c3C(=O)N(C3CCC(=O)NC3=O)C4=O)cc2)cc1]

Aspect 26. The compound of aspect 1, having a structure represented by a formula:

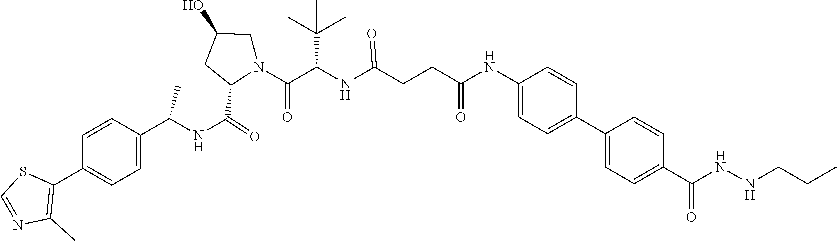 [SMILES: CCCNNC(=O)c1ccc(-c2ccc(NC(=O)CCC(=O)N[C@H](C(=O)N3C[C@H](O)C[C@H]3C(=O)N[C@@H](C)c3ccc(-c4scnc4C)cc3)C(C)(C)C)cc2)cc1]

Aspect 27. The compound of aspect 1, having a structure represented by a formula:

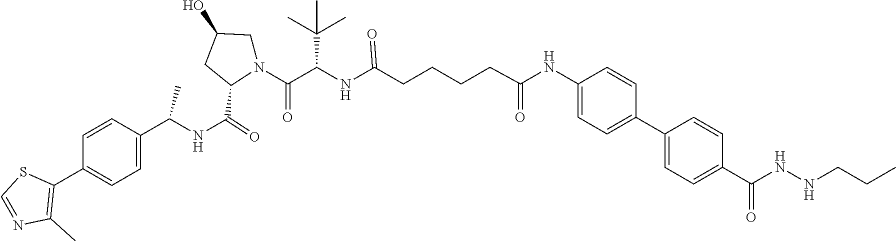 [SMILES: CCCNNC(=O)c1ccc(-c2ccc(NC(=O)CCCCC(=O)N[C@H](C(=O)N3C[C@H](O)C[C@H]3C(=O)N[C@@H](C)c3ccc(-c4scnc4C)cc3)C(C)(C)C)cc2)cc1]

Aspect 28. The compound of aspect 1, having a structure represented by a formula:

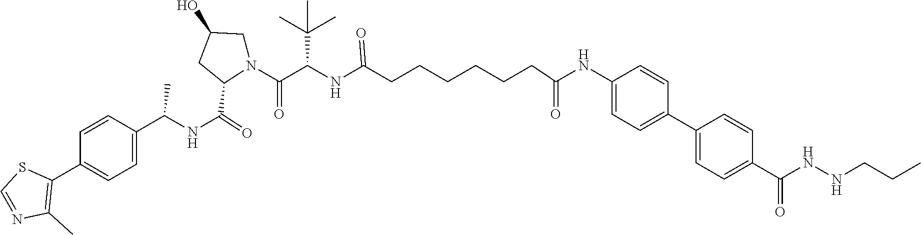 [SMILES: CCCNNC(=O)c1ccc(-c2ccc(NC(=O)CCCCCCC(=O)N[C@H](C(=O)N3C[C@H](O)C[C@H]3C(=O)N[C@@H](C)c3ccc(-c4scnc4C)cc3)C(C)(C)C)cc2)cc1]

Aspect 29. The compound of aspect 1, having a structure represented by a formula:

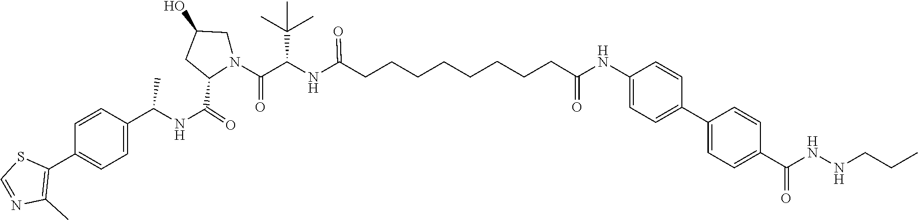 [SMILES: CCCNNC(=O)c1ccc(-c2ccc(NC(=O)CCCCCCCCC(=O)N[C@H](C(=O)N3C[C@H](O)C[C@H]3C(=O)N[C@@H](C)c3ccc(-c4scnc4C)cc3)C(C)(C)C)cc2)cc1]

Aspect 30. The compound of aspect 1, having a structure represented by a formula:

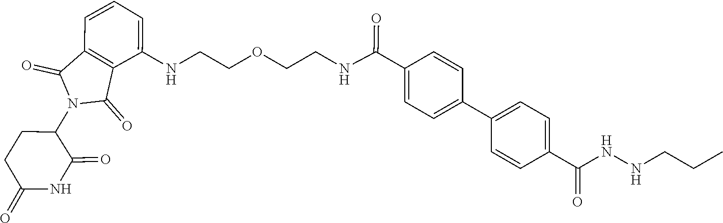 [SMILES: CCCNNC(=O)c1ccc(-c2ccc(C(=O)NCCOCCNc3cccc4c3C(=O)N(C3CCC(=O)NC3=O)C4=O)cc2)cc1]

Aspect 31. The compound of aspect 1, having a structure represented by a formula:

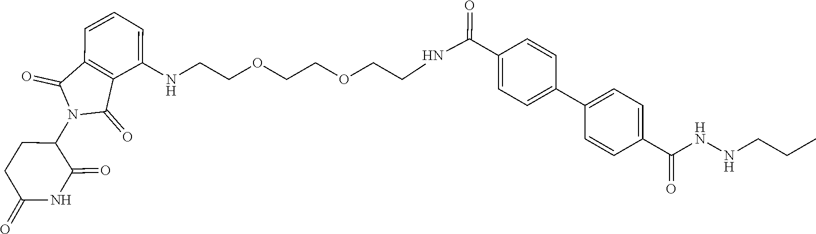 [SMILES: CCCNNC(=O)c1ccc(-c2ccc(C(=O)NCCOCCOCCNc3cccc4c3C(=O)N(C3CCC(=O)NC3=O)C4=O)cc2)cc1]

Aspect 32. The compound of aspect 1, having a structure represented by a formula:

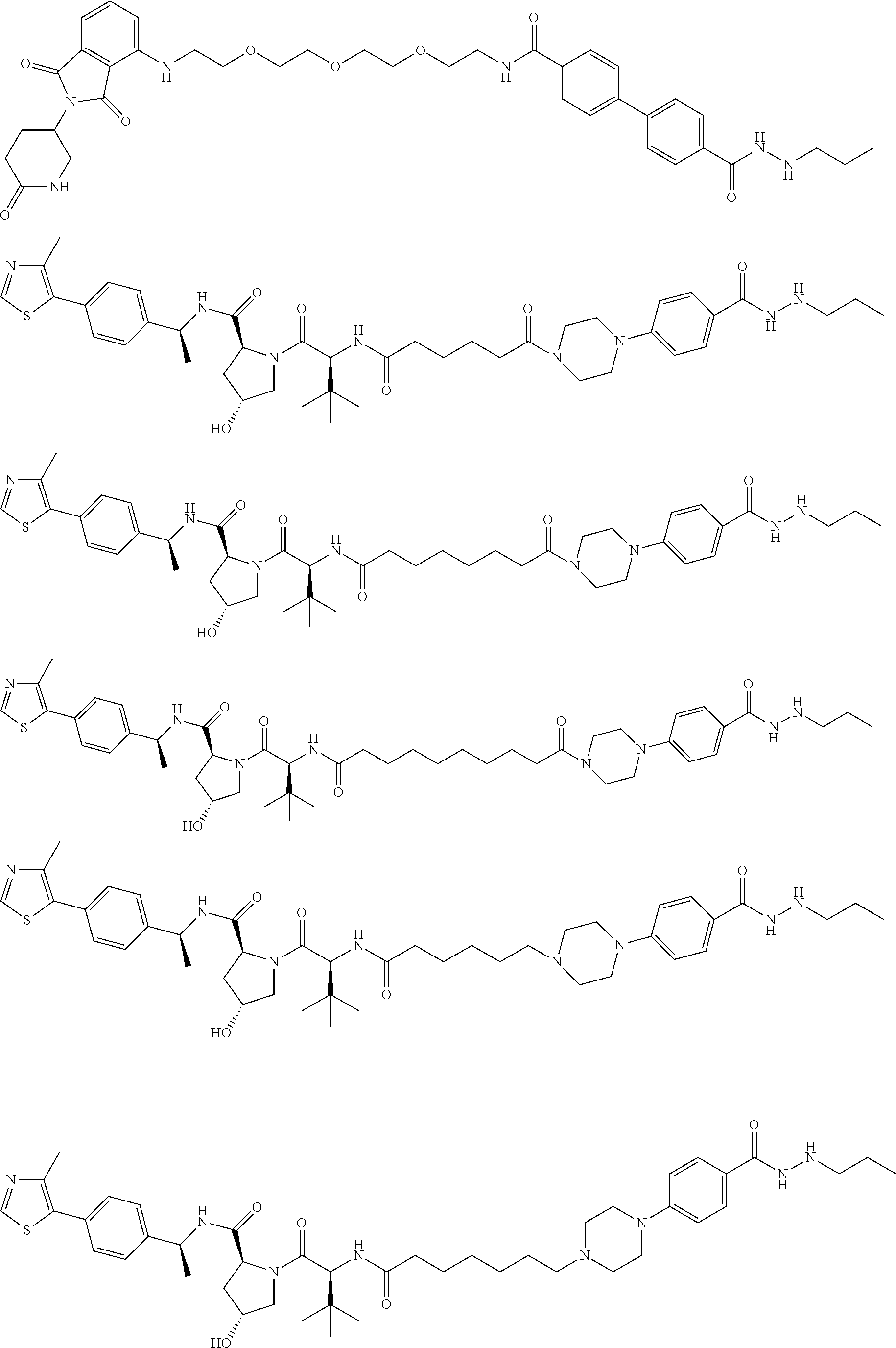

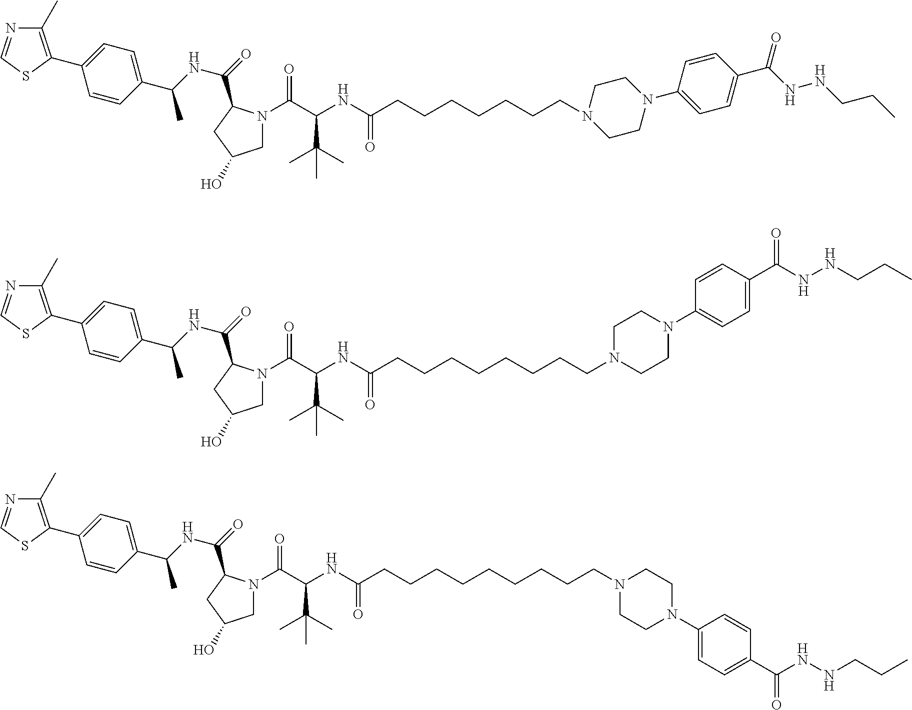 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1 ; CCCNNC(=O)c1ccc(N2CCN(CCCCCCCCC(=O)N[C@H](C(=O)N3C[C@H](O)C[C@H]3C(=O)N[C@@H](C)c3ccc(-c4scnc4C)cc3)C(C)(C)C)CC2)cc1 ; CCCNNC(=O)c1ccc(N2CCN(CCCCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

Aspect 33. The compound of aspect 1, having a structure represented by a formula:

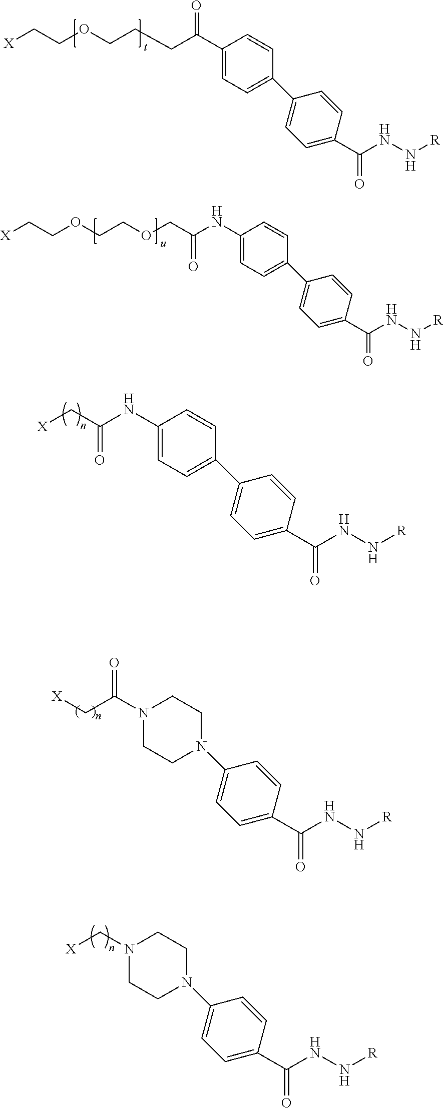

wherein
   X is the E3 ligase targeting moiety;
   t is 1, 2, or 3;
   u is 1, 2, or 3; and
   n is an integer from 1 to 10.
      Aspect 34. The compound of aspect 33, wherein R is a propyl group.
      Aspect 35. The compound of aspects 33 or 34, wherein X is

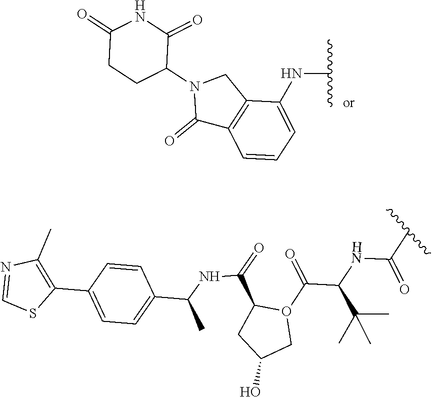

Aspect 36. A pharmaceutical composition comprising a therapeutically effective amount of a compound of any of aspects 1-35, or a pharmaceutically acceptable salt, solvate, or polymorph thereof, and a pharmaceutically acceptable carrier.
Aspect 37. A method for the treatment of a disorder in a mammal, comprising the step of administering to the mammal a therapeutically effective amount of at least one compound of any of aspects 1-35, or a pharmaceutically acceptable salt thereof, or the pharmaceutical composition of aspect 36.
Aspect 38. The method of aspect 37, wherein the mammal is a human.
Aspect 39. The method of aspect 37, wherein the mammal has been diagnosed with a need for treatment of the disorder prior to the administering step.
Aspect 40. The method of aspect 37, further comprising the step of identifying a mammal in need of treatment of the disorder.
Aspect 41. The method of aspect 37, wherein the disorder is selected from breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof.
Aspect 42. A method for inhibiting the activity of at least one histone deacetylase enzyme in a mammal, comprising the step of administering to the mammal a therapeutically effective amount of at least one compound of any of aspects 1-35, or a pharmaceutically acceptable salt thereof, or the pharmaceutical composition of aspect 36.
Aspect 43. The method of aspect 42, wherein the mammal is a human.
Aspect 44. The method of aspect 42, wherein the histone deacetylase enzyme is histone deacetylase 3 (HDAC3).
Aspect 45. The method of aspect 44, wherein the compound exhibits an IC₅₀ of less than 0.55 μM for HDAC3.
Aspect 46. The method of aspect 44, wherein the compound exhibits an IC₅₀ of less than 0.35 μM for HDAC3.
Aspect 47. The method of aspect 44, wherein the compound exhibits an IC₅₀ of less than 0.1 μM for HDAC3.
Aspect 48. The method of any of aspects 44-47, wherein the compound exhibits a lower IC₅₀ for HDAC3 than for histone deacetylase 2 (HDAC2).
Aspect 49. The method of aspect 48, wherein the IC₅₀ for HDAC2 is from about 4 to about 10 times the IC₅₀ for HDAC3.
Aspect 50. The method of any of aspects 44-47, wherein the compound exhibits a lower IC₅₀ for HDAC3 than for histone deacetylase 1 (HDAC1).
Aspect 51. The method of aspect 50, wherein the IC₅₀ for HDAC1 is from about 1.8 to about 3 times the IC₅₀ for HDAC3.
Aspect 52. The method of aspect 42, wherein the mammal has been diagnosed with a need for inhibiting the activity of at least one histone deacetylase enzyme prior to the administering step.
Aspect 53. The method of aspect 42, further comprising the step of identifying a mammal with a need for inhibiting the activity of at least one histone deacetylase enzyme.
Aspect 54. A kit comprising at least one compound of any of aspects 1-35, or a pharmaceutically acceptable salt thereof, and one or more of:
a. at least one agent known to increase the activity of at least one histone deacetylase enzyme;
b. at least one agent known to decrease the activity of at least one histone deacetylase enzyme; and
c. at least one agent known to treat breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof.
   Aspect 55. The kit of aspect 54, further comprising instructions for treating breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof
   Aspect 56. The method of aspect 54, wherein the at least one compound and the at least one agent are co-formulated.
   Aspect 57. The method of aspect 54, wherein the at least one compound and the at least one agent are co-packaged.
   Aspect 58. A method for synthesizing a compound of Formula I, the method comprising:
a. (i) reacting a compound having formula II with an aldehyde in a first solvent to produce and (ii) adding a reducing agent in a second solvent to produce a compound of formula III;

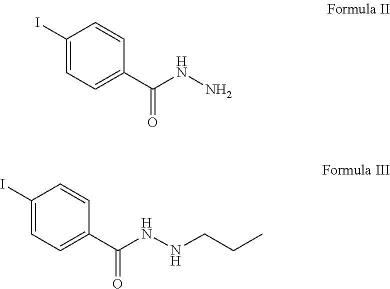

b. reacting the compound of formula III with a protecting group source and a first base in a third solvent to produce a compound of formula IV;

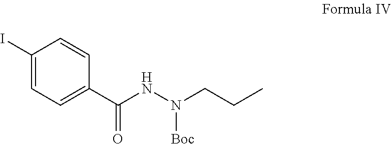 [SMILES: CCCN(NC(=O)c1ccc(I)cc1)C(=O)OC(C)(C)C]

c. reacting the compound of formula IV with a substituted aromatic compound, a first catalyst, and a second base in a fourth solvent at a first temperature to produce a compound of formula V;

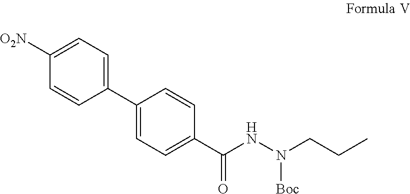 [SMILES: CCCN(NC(=O)c1ccc(-c2ccc([N+](=O)[O-])cc2)cc1)C(=O)OC(C)(C)C]

d. reacting the compound of formula V with a second catalyst and a hydrogenation agent in a fifth solvent to produce a compound of formula VI;

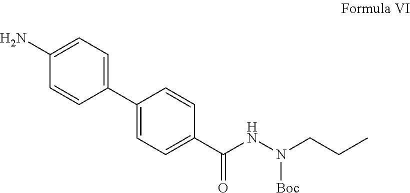 [SMILES: CCCN(NC(=O)c1ccc(-c2ccc(N)cc2)cc1)C(=O)OC(C)(C)C]

e. (i) reacting the compound of formula VI with an anhydride and a third base in a sixth solvent and (ii) followed by addition of a first acid to produce a compound of formula VII,

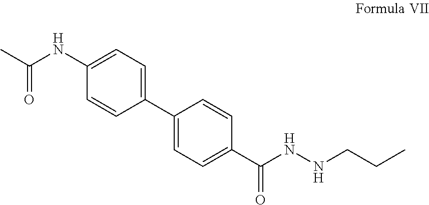 [SMILES: CCCNNC(=O)c1ccc(-c2ccc(NC(C)=O)cc2)cc1]

f. (i) reacting the compound of formula VII with an HDAC-targeting moiety of formula VIII or IX, a coupling agent, and a fourth base in a seventh solvent, (ii) followed by addition of a second acid to produce the compound of formula I;

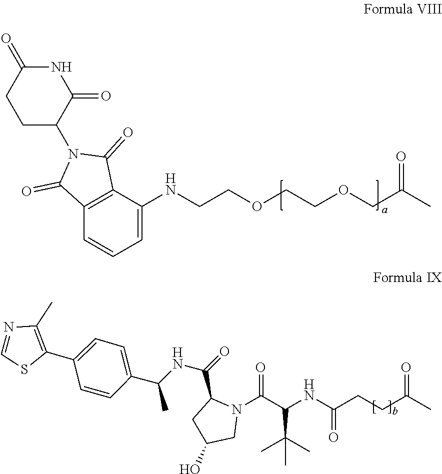

wherein a is from 1 to 3 and b is from 1 to 7.
   Aspect 59. The method of aspect 58, wherein the aldehyde comprises propionaldehyde.
   Aspect 60. The method of aspect 58 or 59, wherein the first solvent comprises methanol, ethanol, isopropanol, dichloromethane, tetrahydrofuran, 1,4-dioxane, or a combination thereof.
   Aspect 61. The method of any of aspects 58-60, wherein the reducing agent comprises sodium borohydride, sodium triacetoxyborohydride, sodium cyanoborohydride, or a combination thereof.
   Aspect 62. The method of any of aspects 58-61, wherein the second solvent comprises methanol, ethanol, isopropanol, methylene chloride, tetrahydrofuran, 1,4-dioxane, or a combination thereof.
   Aspect 63. The method of any of aspects 58-62, wherein the protecting group source comprises di-tert-butyl-dicarbonate.
   Aspect 64. The method of any of aspects 58-63, wherein the first base comprises triethylamine, N,N-diisopropylethylamine, N-methylmorpholine, pyridine, 2,6-lutidine, or a combination thereof.
   Aspect 65. The method of any of aspects 58-64, wherein the third solvent comprises dichloromethane, tetrahydrofuran, 1,4-dioxane, or a combination thereof.
   Aspect 66. The method of any of aspects 58-65, wherein the substituted aromatic compound comprises 4-nitrophenylboronic acid.
   Aspect 67. The method of any of aspects 58-66, wherein the first catalyst comprises Pd(PPh₃)₄.
   Aspect 68. The method of any of aspects 58-67, wherein the second base comprises sodium carbonate, potassium carbonate, cesium carbonate, trimethylamine, N,N-diisopropylethylamine, N-methylmorpholine, pyridine, 2,6-lutidine, or a combination thereof.
   Aspect 69. The method of any of aspects 58-68, wherein the fourth solvent comprises toluene, ethanol, water, tetrahydrofuran, 1,4-dioxane, dimethylformamide, or a combination thereof.
   Aspect 70. The method of any of aspects 58-69, wherein the first temperature is from about 60 to about 120° C.
   Aspect 71. The method of any of aspects 58-70, wherein the second catalyst comprises Pd/C.
   Aspect 72. The method of any of aspects 58-71, wherein the hydrogenation agent comprises H₂.
   Aspect 73. The method of any of aspects 58-72, wherein the fifth solvent comprises ethyl acetate, methanol, ethanol, isopropanol, tetrahydrofuran, 1,4-dioxane, or a combination thereof.
   Aspect 74. The method of any of aspects 58-73, wherein the E3 ligase-targeting moiety comprises Formula VIII and a is 1.
   Aspect 75. The method of any of aspects 58-73, wherein the E3 ligase-targeting moiety comprises Formula VIII and a is 2.
   Aspect 76. The method of any of aspects 58-73, wherein the E3 ligase-targeting moiety comprises Formula VIII and a is 3.
   Aspect 77. The method of any of aspects 58-73, wherein the E3 ligase-targeting moiety comprises Formula IX and b is 1.
   Aspect 78. The method of any of aspects 58-73, wherein the E3 ligase-targeting moiety comprises Formula IX and b is 3.
   Aspect 79. The method of any of aspects 58-73, wherein the E3 ligase-targeting moiety comprises Formula IX and b is 5.
   Aspect 80. The method of any of aspects 58-73, wherein the E3 ligase-targeting moiety comprises Formula IX and b is 7.
   Aspect 81. The method of any of aspects 58-80, wherein the anhydride comprises acetic anhydride.
   Aspect 82. The method of any of aspects 58-81, wherein the third base comprises triethylamine, N,N-diisopropylethylamine, N-methylmorpholine, pyridine, 2,6-lutidine, or a combination thereof.
   Aspect 83. The method of any of aspects 58-82, wherein the sixth solvent comprises dichloromethane, tetrahydrofuran, 1,4-dioxane, dimethylformamide, or a combination thereof.
   Aspect 84. The method of any of aspects 58-83, wherein the first acid comprises trifluoroacetic acid, methanesulfonic acid, p-toluenesulfonic acid, hydrochloric acid, or a combination thereof.
   Aspect 85. The method of any of aspects 58-84, wherein the coupling agent comprises hexafluorophosphate azabenzotriazole tetramethyl uronium (HATU), 1-ethyl-3-(3-dimethylaminopropyl)carbodiimide, N,N′-dicyclohexylcarbodiimide, propanephosphonic acid anhydride, benzotriazole-1-yl-oxy-tris-pyrrolidino-phosphonium hexafluorophosphate, or a combination thereof.
   Aspect 86. The method of any of aspects 58-85, wherein the fourth base comprises trimethylamine, N,N-diisopropylethylamine, N-methylmorpholine, pyridine, 2,6-lutidine, or a combination thereof.
   Aspect 87. The method of any of aspects 58-86, wherein the seventh solvent comprises dichloromethane, tetrahydrofuran, 1,4-dioxane, dimethylformamide, or a combination thereof.
   Aspect 88. The method of any of aspects 58-87, wherein the second acid comprises trifluoroacetic acid, methanesulfonic acid, p-toluenesulfonic acid, hydrochloric acid, or a combination thereof.
   Aspect 89. A method for synthesizing a compound of Formula I, the method comprising:
a. (i) reacting a compound having formula II with an aldehyde in a first solvent to produce and (ii) adding a reducing agent in a second solvent to produce a compound of formula III;

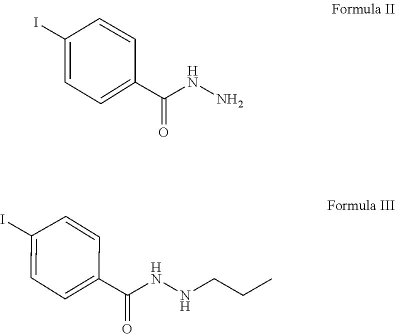

b. reacting the compound of formula III with a protecting group source and a first base in a third solvent to produce a compound of formula IV;

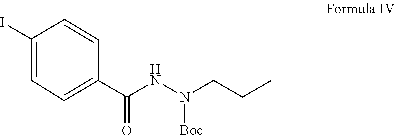 [SMILES: CCCN(NC(=O)c1ccc(I)cc1)C(=O)OC(C)(C)C]

c. reacting the compound of formula IV with an aromatic acid, a first catalyst, and a second base in a third solvent at a first temperature to produce a compound of formula X;

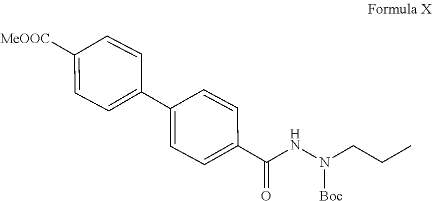 [SMILES: CCCN(NC(=O)c1ccc(-c2ccc(C(=O)OC)cc2)cc1)C(=O)OC(C)(C)C]

d. reacting the compound of formula X with a third base in a fourth solvent at a second temperature to produce a compound of formula XI;

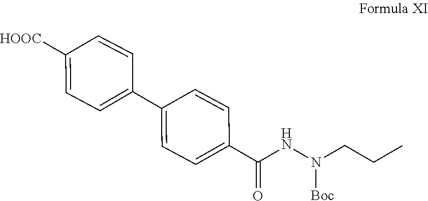 [SMILES: CCCN(NC(=O)c1ccc(-c2ccc(C(=O)O)cc2)cc1)C(=O)OC(C)(C)C]

e. reacting the compound of formula XI with an HDAC-targeting moiety of formula XII with a fourth base and a coupling agent in a fifth solvent followed by addition of an acid to produce the compound of formula I

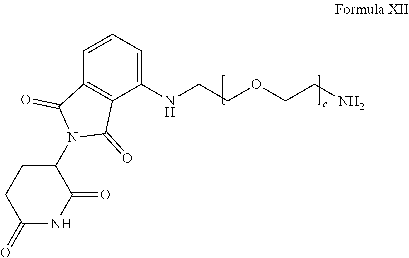

wherein c is from 1 to 3.
   Aspect 90. The method of aspect 89, wherein the aldehyde comprises propionaldehyde.
   Aspect 91. The method of aspect 89 or 90, wherein the first solvent comprises methanol, ethanol, isopropanol, dichloromethane, tetrahydrofuran, 1,4-dioxane, or a combination thereof.
   Aspect 92. The method of any of aspects 89-91, wherein the reducing agent comprises sodium borohydride, sodium triacetoxyborohydride, sodium cyanoborohydride, or a combination thereof.
   Aspect 93. The method of any of aspects 89-92, wherein the second solvent comprises methanol, ethanol, isopropanol, methylene chloride, tetrahydrofuran, 1,4-dioxane, or a combination thereof.
   Aspect 94. The method of any of aspects 89-93, wherein the protecting group source comprises di-tert-butyl-dicarbonate.
   Aspect 95. The method of any of aspects 89-94, wherein the first base comprises trimethylamine, N,N-diisopropylethylamine, N-methylmorpholine, pyridine, 2,6-lutidine, or a combination thereof.
   Aspect 96. The method of any of aspects 89-95, wherein the third solvent comprises dichloromethane, tetrahydrofuran, 1,4-dioxane, or a combination thereof.
   Aspect 97. The method of any of aspects 89-96, wherein the aromatic acid comprises (4-(methoxycarbonyl)phenyl)boronic acid.
   Aspect 98. The method of any of aspects 89-97, wherein the first catalyst comprises Pd(PPh₃)₄.
   Aspect 99. The method of any of aspects 89-98, wherein the second base comprises sodium carbonate, potassium carbonate, cesium carbonate, trimethylamine, N,N-diisopropylethylamine, N-methylmorpholine, pyridine, 2,6-lutidine, or a combination thereof.
   Aspect 100. The method of any of aspects 89-99, wherein the third solvent comprises toluene, ethanol, water, tetrahydrofuran, 1,4-dioxane, dimethylformamide, or a combination thereof.
   Aspect 101. The method of any of aspects 89-100, wherein the first temperature is from about 60 to about 120° C.
   Aspect 102. The method of any of aspects 89-101, wherein the third base comprises lithium hydroxide, sodium hydroxide, potassium hydroxide, potassium carbonate, or a combination thereof.
   Aspect 103. The method of any of aspects 89-102, wherein the fourth solvent comprises methanol, ethanol, isopropanol, tetrahydrofuran, 1,4-dioxane, water, or a combination thereof.
   Aspect 104. The method of any of aspects 89-103, wherein the second temperature is from about 25 to about 80° C.
   Aspect 105. The method of any of aspects 89-104, wherein c is 1.
   Aspect 106. The method of any of aspects 89-104, wherein c is 2.
   Aspect 107. The method of any of aspects 89-104, wherein c is 3.
   Aspect 108. The method of any of aspects 89-107, wherein the fourth base comprises trimethylamine, N,N-diisopropylethylamine, N-methylmorpholine, pyridine, 2,6-lutidine, or a combination thereof.
   Aspect 109. The method of any of aspects 89-108, wherein the coupling agent comprises hexafluorophosphate azabenzotriazole tetramethyl uronium (HATU), 1-ethyl-3-(3-dimethylaminopropyl)carbodiimide, N,N′-dicyclohexylcarbodiimide, propanephosphonic acid anhydride, benzotriazole-1-yl-oxy-tris-pyrrolidino-phosphonium hexafluorophosphate, or a combination thereof.
   Aspect 110. The method of any of aspects 89-109, wherein the fifth solvent comprises dichloromethane, tetrahydrofuran, 1,4-dioxane, dimethylformamide, or a combination thereof.
   Aspect 111. The method of any of aspects 89-110, wherein the acid comprises trifluoroacetic acid, methanesulfonic acid, p-toluenesulfonic acid, hydrochloric acid, or a combination thereof.
   Aspect 112. A method for synthesizing a compound of Formula I, the method comprising:

reacting the compound of formula X with an HDAC-targeting moiety of formula IX to produce the compound of formula I;

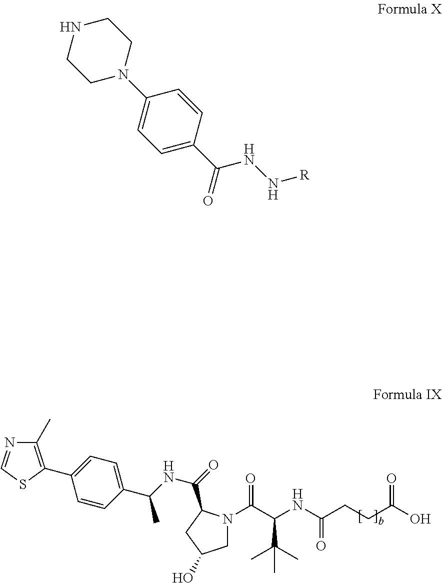

wherein b is from 1 to 10, and
R comprises a substituted or unsubstituted C1-C6 linear or branched alkyl group, a C3-C6 substituted or unsubstituted cycloalkyl group, or a combination thereof.
   Aspect 113. A method for synthesizing a compound of Formula I, the method comprising:

reacting the compound of formula X with an HDAC-targeting moiety of formula IX to produce the compound of formula I;

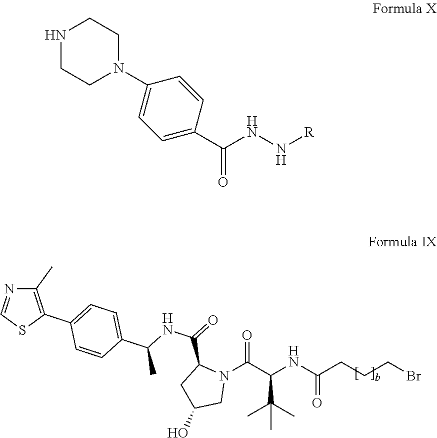

wherein b is from 1 to 10, and

R comprises a substituted or unsubstituted C1-C6 linear or branched alkyl group, a C3-C6 substituted or unsubstituted cycloalkyl group, or a combination thereof.

### REFERENCES

Alam, M. S. et al, Science Translational Medicine, 2016, 8, 326ra323-326ra323.
An, Z. et al., Protein Cell, 2019, 10, 606-609.
Angiolilli, C. et al, Ann Rheum Dis, 2017, 76, 277-285.
Aune, S. E. et al, J Mol Cell Cardiol, 2014, 72, 138-145.
Bhaskara, S. et al., Cancer Cell, 2010, 18, 436-447.
Caslini, C. et al., Oncogene, 2019, 38, 6599-6614.
Chen, X. et al, Proc Natl Acad Sci USA, 2012, 109, E2865-2874.
Christensen, D. P. et al, Proc Natl Acad Sci USA, 2014, 111, 1055-1059.
Conti, C. et al., Cancer Res., 2010, 70, 4470-4480.
Cui, Z. et al., Med. Sci. Monit., 2018, 24, 2456-2464.
Dirice, E. et al, J Biol Chem, 2017, 292, 17598-17608.
Emmett, M. J. et al, Nat Rev Mol Cell Biol, 2019, 20, 102-115.
Fass, D. M. et al, Neuroscience, 2014, 264, 112-130.
Felice, C. et al, Aliment Pharmacol Ther, 2015, 41, 26-38.
Grabiec, A. M. et al, Crit Rev Immunol, 2011, 31, 233-263.
Graff, J. et al, Annu Rev Pharmacol Toxicol, 2013, 53, 311-330.
Graff, J. et al, Cell, 2014, 156, 261-276.
Gryder, B. E. et al., Fut. Med. Chem., 2012, 4, 505-524.
Gryder, B. E. et al., Nat. Commun., 2019, 10, 3004.
Guha, M., Nat Rev Drug Discov, 2015, 14, 225-226.
Jiang, Z. et al., Lancet Oncol., 2019, 20, 806-815.
Kazantsev, A. G. et al, Nat Rev Drug Discov, 2008, 7, 854-868.
Khan, S. et al., Nat. Med., 2019, 25, 1938-1947.
Kwak, S. M. et al., Cells, 2019, 8, 930.
Lehmann, L. H. et al, Cell Mol Life Sci, 2014, 71, 1673-1690.
Leus, N. G. et al, Curr Opin Chem Biol, 2016, 33, 160-168.
Li, X. et al., J. Med. Chem., 2018, 61, 2589-2603.
Lundh, M. et al, Diabetes Obes Metab, 2015, 17, 703-707.
Matteucci, E. et al., Mol. Cancer Res., 2007, 5, 833-845.
McClure, J. J. et al., J. Med. Chem., 2016, 59, 9942-9959.
Mukhamejanova, Z. et al., Curr. Med. Chem., 2020, DOI: 10.2174/0929867327666200312112412.
Muller, B. M. et al., BMC Cancer, 2013, 13, 215.
Paiva, S.-L. et al., Curr. Opin. Chem. Biol., 2019, 50, 111-119.
Ridolfi, E. et al., Br. J. Cancer, 2008, 99, 1623-1634.
Sandi, C. et al, Neurobiol Dis, 2011, 42, 496-505.
Smalley, J. P. et al., Chem. Commun., 2020, 56, 4476-4479.
Summers, A. R. et al., J. Clin. Invest., 2013, 123, 3112-3123.
Sun, Z. et al., Mol. Cell, 2013, 52, 769-782.
Tang, X. et al., Nucleic Acids Res., 2020, 48, 2912-2923.
Wagner, F. F. et al, Chem Sci, 2015, 6, 804-815.
Wagner, F. F. et al., Neurotherapeutics, 2013, 10, 589-604.
Wang, Y. et al., Chem. Biol., 2015, 22, 273-284.
Watson, P. J. et al., Nature, 2012, 481, 335-340.
Wells, C. E. et al., PLoS One, 2013, 8, e68915.
West, A. C. et al., J Clin Invest, 2014, 124, 30-39.
Wu H. et al., J. Med. Chem., 2019, 62, 7042-7057.
Xu, C. et al, Chem Biol, 2009, 16, 980-989.
Yang, H. et al., Chem. Commun., 2019, 55, 14848-14851.
Yang, H. et al., Mol Cancer Ther, 2013, 12, 610-620.
Yang, K. et al., Bioorg. Med. Chem. Lett., 2018, 28, 2493-2497.
Yang, K. et al., ACS Med. Chem. Lett., 2020, 11, 575-581.
Yang, X. J. et al., Mol. Cell, 2008, 31, 449-461.
Yang, X. J. et al, Nat Rev Mol Cell Biol, 2008, 9, 206-218.
Yardley, D. A. et al., J. Clin. Oncol., 2013, 31, 2128-2135.
Zhang, L. et al, Med. Res. Rev., 2015, 35, 63-84.
Zhang, X. et al., Chem. Commun., 2019, 55, 14765-14768.
Zhang, X. et al., Eur. J. Med. Chem., 2020, 192, 112186.
Zou, Y. et al., Cell Biochem. Funct., 2019, 37, 21-30.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthesis and Characterization

### Synthesis Strategy

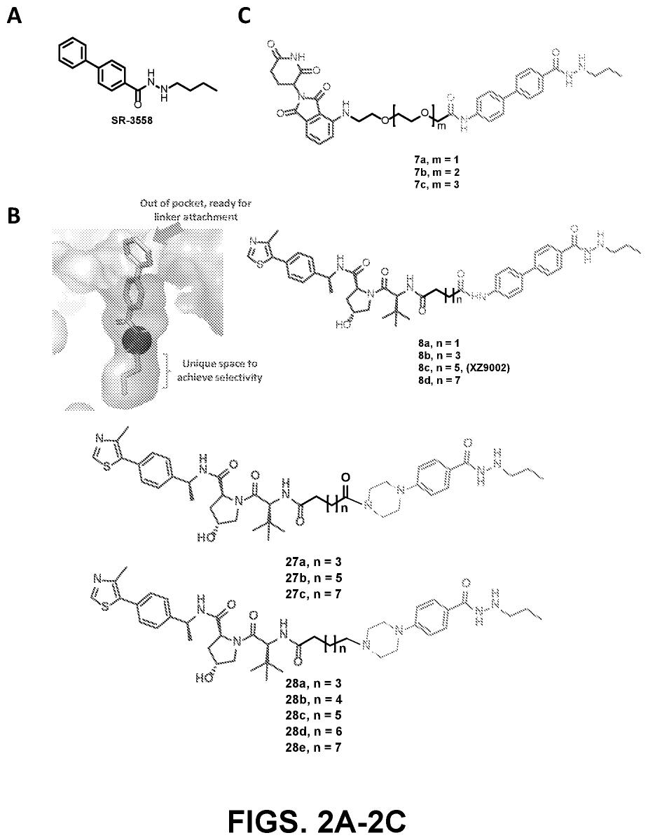

SR-3558 had previously been identified as a selective inhibitor of class I HDACs with a novel benzoylhydrazide zinc-binding group (ZBG) (FIG. 2A). Molecular docking studies using AutoDock Vina were conducted wherein it was found that the alkane tail in the ZBG occupies the bottom space of binding pocket in class I HDACs (FIG. 2B). This binding mode explained the unique selectivity of SR-3558, since other HDACs such as HDAC6 do not have this extra space. Moreover, the lowest binding energy conformation of SR-3558 also revealed that the terminal phenyl ring is exposed to solvent, which can be utilized for linker attachment to synthesize PROTACs. Based on these findings, we designed PROTACs 7a-c and 8a-d which recruit CRBN and VHL E3 ligases, respectively (FIG. 2C).

To efficiently synthesize the designed PROTACs, a novel synthetic strategy is required, since the reported synthetic methods of benzoylhydrazide-containing HDACi involve a multi-step procedure using advanced benzoic acid/ester and hydrazine as starting materials. Furthermore, due to the structural complexity of designed PROTACs, it can be quiet challenging to introduce ZBG in the late stage. Considering these factors, we developed a new strategy to build an advanced intermediate 13 in four steps with an excellent yield.

Briefly, 4-iodobenzohydrazide (9) was converted to 10 through a reductive amidation with propionaldehyde. The basic nitrogen in 10 was protected with Boc to give 11, which was coupled with 4-nitrophenylboronic acid through a Suzuki reaction followed by reduction of the nitro group in 12 via hydrogenolysis to afford precursor 13. The amidation between 13 and acid 14 in the presence of HATU, as well as the following cleavage of Boc group under acidic condition went smoothly to afford 8c (XZ9002). PROTACs 7a-c and 8a-d were synthesized using the same method (Scheme 1 and Scheme 2) and YX series compounds were synthesized according to scheme 3:

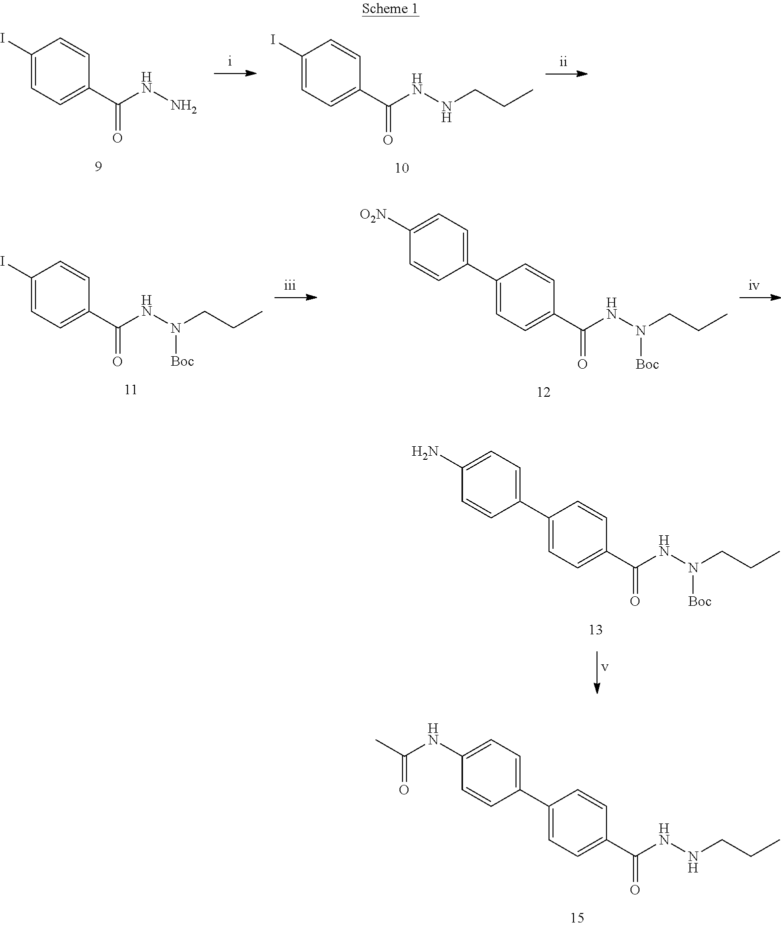 [SMILES: NNC(=O)c1ccc(I)cc1 ; CCCNNC(=O)c1ccc(I)cc1 ; CCCN(NC(=O)c1ccc(I)cc1)C(=O)OC(C)(C)C ; CCCN(NC(=O)c1ccc(-c2ccc([N+](=O)[O-])cc2)cc1)C(=O)OC(C)(C)C ; CCCN(NC(=O)c1ccc(-c2ccc(N)cc2)cc1)C(=O)OC(C)(C)C ; CCCNNC(=O)c1ccc(-c2ccc(NC(C)=O)cc2)cc1]

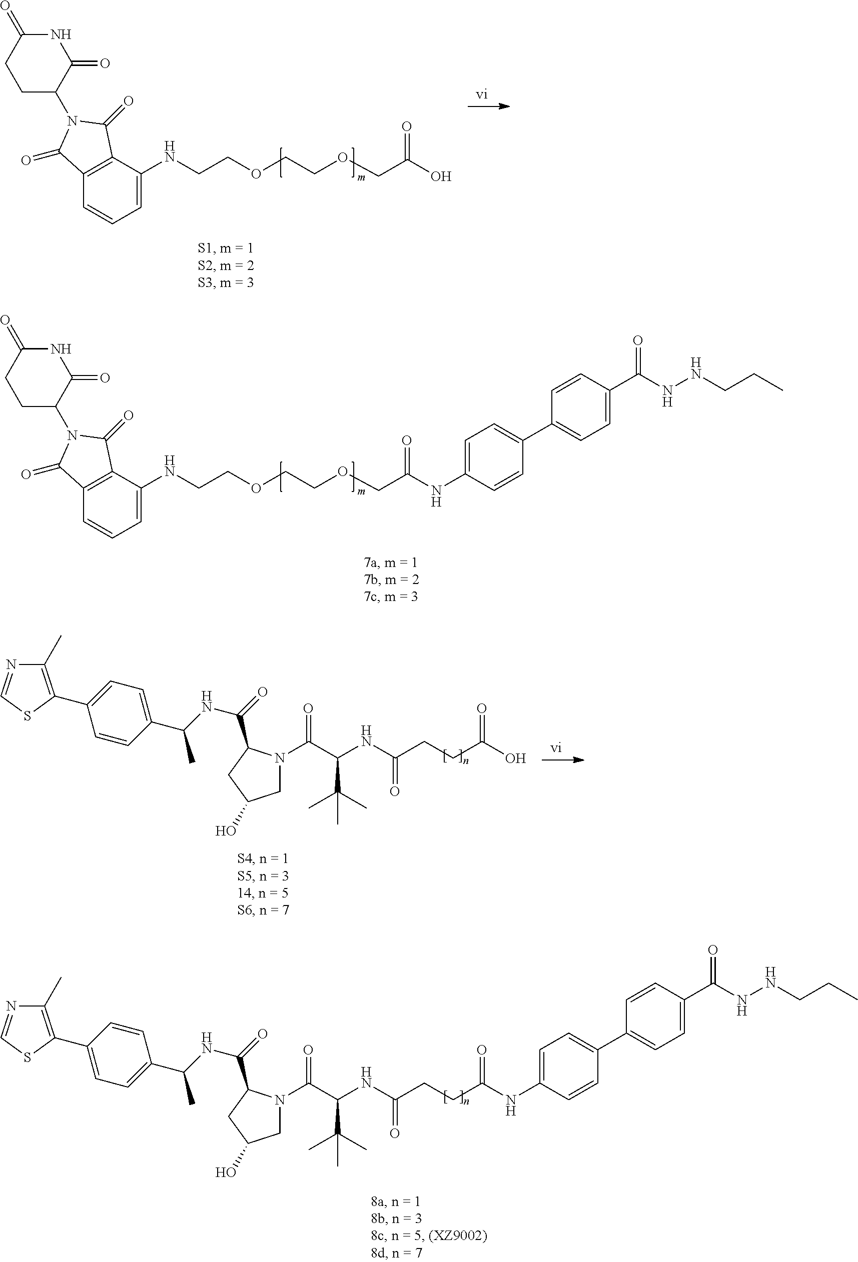

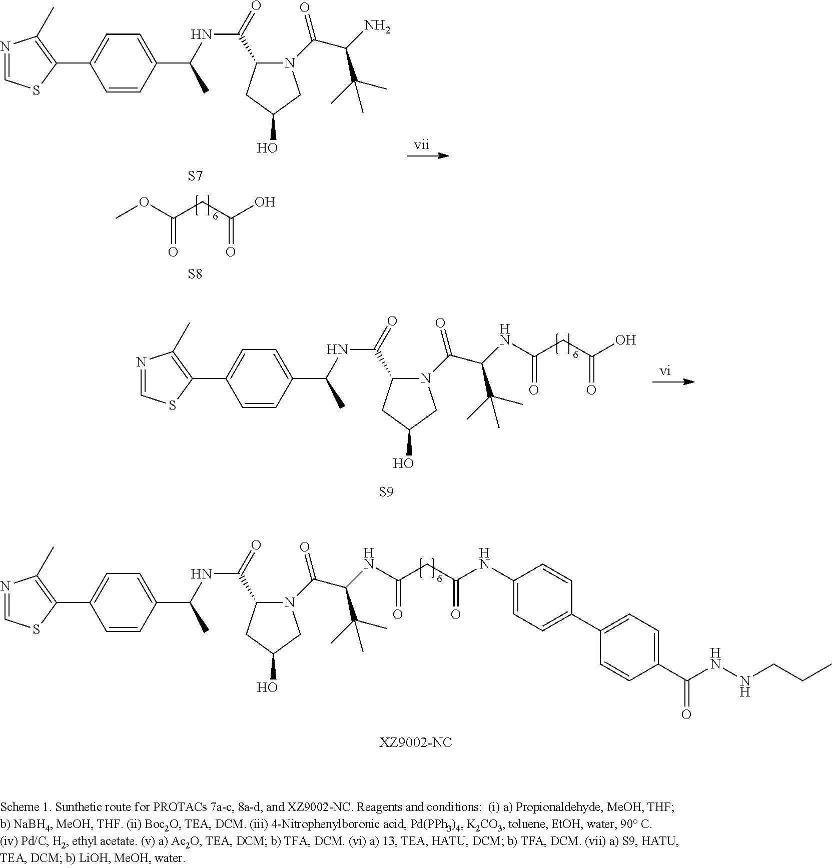

### Reagents

THF, DCM, and toluene were obtained via a solvent purification system by filtering through two columns packed with activated alumina and 4 Å molecular sieves, respectively. All other chemicals obtained from commercial sources were used without further purification. Flash chromatography was performed using silica gel (230-400 mesh) as the stationary phase. Reaction progress was monitored by thin-layer chromatography (silica-coated glass plates) and visualized by UV light, and/or by LC-MS. ¹H NMR spectra were recorded in CDCl3, DMSO-d₆, or CD₃OD at 600 MHz. Chemical shifts δ are given in ppm using tetramethylsilane as an internal standard. Multiplicities of NMR signals are designated as singlet (s), broad singlet (br s), doublet (d), doublet of doublets (dd), triplet (t), quartet (q), and multiplet (m). All final compounds for biological testing were of ≥95.0% purity as analyzed by LC-MS, performed on an Advion AVANT LC system with the expression CMS using a Thermo Accucore™ Vanquish™ C18+ UHPLC Column (1.5 μm, 50×2.1 mm) at 40° C. Gradient elution was used for UHPLC with a mobile phase of acetonitrile and water containing 0.1% formic acid.

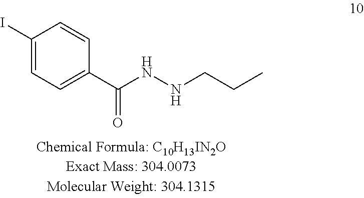

### 4-Iodo-N′-propylbenzohydrazide (10)

A mixture of compound 9 (262 mg, 1.0 mmol) and propionaldehyde (87 mg, 1.5 mmol) in MeOH-THF (1:1, v/v, 10 mL) was stirred at room temperature for 2 h. The solvent was removed under reduced pressure and the residue was dissolved in MeOH-THF (1:1, v/v, 10 mL). The solution was treated with NaBH₄ (60 mg, 1.6 mmol) and stirred for 30 min. Then it was diluted with water and extracted with ethyl acetate. The organic phase was washed with water x 1, brine×1, dried over Na₂SO₄, filtered, and evaporated to dryness. The residue was used directly in the next step (270 mg, yield 89%). ¹H NMR (600 MHz, CDCl₃) δ 7.82 (d, J=8.4 Hz, 2H), 7.49 (d, J=8.5 Hz, 2H), 2.93 (t, J=7.2 Hz, 2H), 1.64-1.53 (m, 2H), 1.00 (t, J=7.2 Hz, 3H). LC-MS (ESI): m/z 305.0 [M+H]⁺.

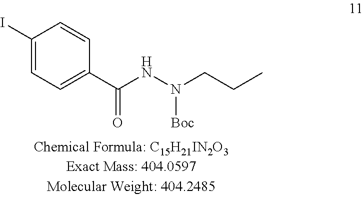 [SMILES: CCCN(NC(=O)c1ccc(I)cc1)C(=O)OC(C)(C)C]

### Tert-Butyl 2-(4-iodobenzoyl)-1-propylhydrazine-1-carboxylate (11)

A mixture of compound 10 (100 mg, 0.32 mmol), Boc₂O (108 mg, 0.50 mmol), and TEA (140 μL, 1.0 mmol) in DCM (5 mL) was stirred at room temperature for 16 h. Then it was diluted with water and extracted with DCM. The organic phase was washed with water×1, brine×1, dried over Na₂SO₄, filtered and evaporated to dryness. The residue was further purified by column chromatography to afford the title compound (123 mg, yield 95%). ¹H NMR (600 MHz, DMSO-d₆) δ 10.58 (s, 1H), 8.00-7.84 (m, 2H), 7.73-7.44 (m, 2H), 3.44-3.34 (m, 2H), 1.61-1.28 (m, 11H), 0.99-0.83 (m, 3H). LC-MS (ESI): m/z 405.1 [M+H]⁺.

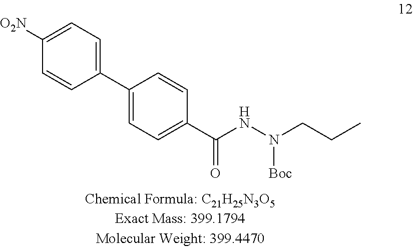 [SMILES: CCCN(NC(=O)c1ccc(-c2ccc([N+](=O)[O-])cc2)cc1)C(=O)OC(C)(C)C]

### Tert-Butyl 2-(4′-nitro-[1,1′-biphenyl]-4-carbonyl)-1-prophylydrazine-1-carboxylate (12)

A mixture of compound 11 (300 mg, 0.74 mmol), 4-nitrophenylboronic acid (150 mg, 0.90 mmol), Pd(PPh₃)₄ (40 mg, 0.03 mmol), and K₂CO₃ (204 mg, 1.48 mmol) was stirred in toluene-EtOH-water (9:1:1, v/v/v, 2 mL) at 90° C. under an argon atmosphere for 16 h. The reaction mixture was cooled to room temperature and poured into water. The resulting solution was extracted with ethyl acetate. The organic phase was washed with water×1, brine×1, dried over Na₂SO₄, filtered and evaporated to dryness. The residue was further purified by column chromatography to afford the title compound (243 mg, yield 82%). ¹H NMR (600 MHz, CDCl₃) δ 8.35 (d, J=8.8 Hz, 2H), 7.94 (d, J=7.9 Hz, 2H), 7.81-7.76 (m, 2H), 7.76-7.62 (m, 2H), 3.61 (t, J=7.2 Hz, 2H), 1.72-1.64 (m, 2H), 1.56-1.39 (m, 9H), 0.98 (t, J=7.2 Hz, 3H). LC-MS (ESI): m/z 400.1 [M+H]⁺.

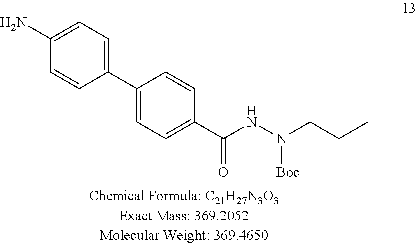 [SMILES: CCCN(NC(=O)c1ccc(-c2ccc(N)cc2)cc1)C(=O)OC(C)(C)C]

### Tert-Butyl 2-(4′-amino-[1,1′-biphenyl]-4-carbonyl)-1-propylhydrazine-1-carboxylate (13)

A mixture of compound 12 (243 mg, 0.61 mmol) and Pd/C (10% w/w, 30 mg) in ethyl acetate (20 mL) was stirred at room temperature for 2 h. After solid was removed by filtration, the filtrate was evaporated to dryness to afford the title compound (225 mg, 100% yield). ¹H NMR (600 MHz, CDCl₃) δ 7.90-7.69 (m, 3H), 7.66-7.59 (m, 2H), 7.50-7.45 (m, 2H), 6.81-6.77 (m, 2H), 3.83 (s, 2H), 3.60 (t, J=7.2 Hz, 2H), 1.71-1.63 (m, 2H), 1.55-1.41 (m, 9H), 0.97 (t, J=7.2 Hz, 3H). LC-MS (ESI): m/z 370.2 [M+H]⁺.

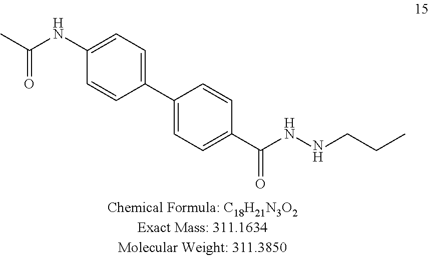 [SMILES: CCCNNC(=O)c1ccc(-c2ccc(NC(C)=O)cc2)cc1]

### N-(4′-(2-propylhydrazine-1-carbonyl)[1,1′-biphenyl]-4-yl)acetamide (15)

A mixture of 13 (15 mg, 0.04 mmol), Ac₂O (4.6 μL, 0.05 mmol), and TEA (45 μL, 0.32 mmol) in DCM (3 mL) was stirred at room temperature for 16 h. The mixture was poured into water and extracted with DCM. The combined organic layers were washed with NH₄Cl (aq.)×1, brine x 1, dried over anhydrous Na₂SO₄, filtered, and concentrated under vacuum. The residue was dissolved in DCM (2 mL) and treated with TFA (0.2 mL). The mixture was stirred at room temperature for 4 h and concentrated under reduced pressure. The crude product was purified by flash column chromatography to afford the title compound (3.9 mg, yield 31%). ¹H NMR (600 MHz, CD₃OD) δ 7.95-7.90 (m, 2H), 7.81-7.76 (m, 2H), 7.72-7.65 (m, 4H), 3.10 (t, J=7.5 Hz, 2H), 2.17 (s, 3H), 1.78-1.67 (m, 2H), 1.06 (t, J=7.5 Hz, 3H). LC-MS (ESI): m/z 312.1 [M+H]⁺.

General Method A for Compounds 7a-c, 8a-d, and XZ9002-NC

Acids S1-6 and 14 were prepared according to a previously published method. A mixture of corresponding acid (1.0 equiv.), amine (1.0 equiv.), HATU (1.05 equiv.) and Et₃N (5.0 equiv.) in DCM was stirred at room temperature for 1 h. The mixture was poured into water and extracted with DCM. The combined organic layers were washed with NH₄Cl (aq.)×1, brine x 1, dried over anhydrous Na₂SO₄, filtered, and concentrated under vacuum. The residue was dissolved in DCM and treated with TFA (20 equiv.). The mixture was stirred at room temperature for 4 h and concentrated under reduced pressure. The crude product was purified by flash column chromatography to afford the desired compound.

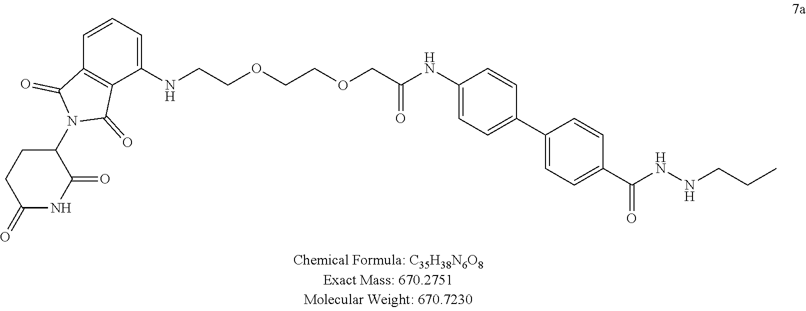 [SMILES: CCCNNC(=O)c1ccc(-c2ccc(NC(=O)COCCOCCNc3cccc4c3C(=O)N(C3CCC(=O)NC3=O)C4=O)cc2)cc1]

### 2-(2-(2-((2-(2,6-dioxopiperidin-3-yl)-1,3-dioxoisoindolin-4-yl)ethoxy)ethoxy)-N-(4′-(2-propylhydrazine-1-carbonyl)-[1,1′-biphenyl]-4-yl)acetamide (7a)

Following the general method above, compound 7a was obtained from S1 and 13 (6.9 mg, yield 38%). ¹H NMR (600 MHz, CD₃OD) δ 7.96 (d, J=8.4 Hz, 2H), 7.73 (d, J=8.5 Hz, 2H), 7.62 (d, J=8.6 Hz, 2H), 7.52 (d, J=8.6 Hz, 2H), 7.48 (dd, J=8.5, 7.1 Hz, 1H), 7.05 (d, J=8.6 Hz, 1H), 6.90 (d, J=7.0 Hz, 1H), 4.96-4.91 (m, 1H), 4.18 (s, 2H), 3.92-3.79 (m, 6H), 3.59-3.54 (m, 2H), 3.32-3.29 (m, 2H), 2.80-2.54 (m, 3H), 2.01-1.96 (m, 1H), 1.86-1.79 (m, 2H), 1.10 (t, J=7.5 Hz, 3H). LC-MS (ESI): m/z 671.3 [M+H]⁺.

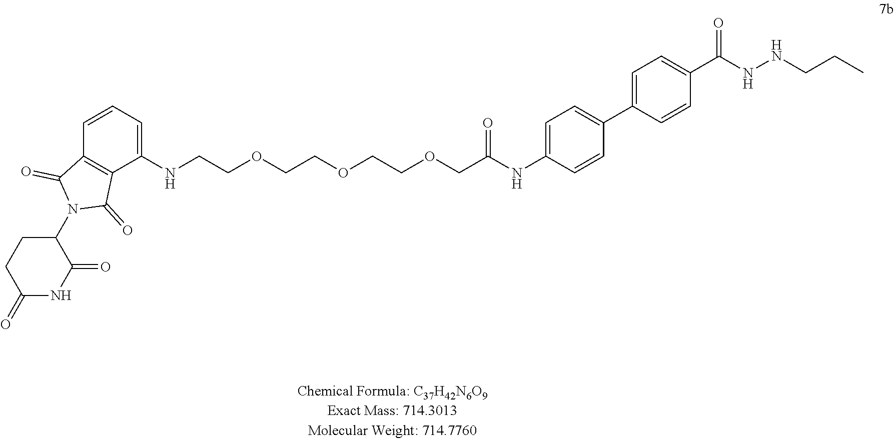 [SMILES: CCCNNC(=O)c1ccc(-c2ccc(NC(=O)COCCOCCOCCNc3cccc4c3C(=O)N(C3CCC(=O)NC3=O)C4=O)cc2)cc1]

### 2-(2-(2-(2-((2-(2,6-dioxopiperidin-3-yl)-1,3-dioxoisoindolin-4-yl)amino)ethoxy)ethoxy)ethoxy)-N-(4′-(20propylhydrazine-1-carbonyl)[1,1′-biphenyl]-4-yl)acetamide (7b)

Following the general method above, compound 7b was obtained from S2 and 13 (7.7 mg, yield 40%). ¹H NMR (600 MHz, CD₃OD) δ 7.94 (d, J=8.4 Hz, 2H), 7.76-7.70 (m, 4H), 7.63-7.57 (m, 2H), 7.44 (dd, J=8.5, 7.1 Hz, 1H), 6.98 (d, J=8.6 Hz, 1H), 6.94 (d, J=7.0 Hz, 1H), 4.99 (dd, J=12.8, 5.5 Hz, 1H), 4.16 (s, 2H), 3.84-3.69 (m, 10H), 3.44 (t, J=5.2 Hz, 2H), 3.32-3.30 (m, 2H), 2.89-2.60 (m, 3H), 2.11-2.01 (m, 1H), 1.89-1.78 (m, 2H), 1.11 (t, J=7.5 Hz, 3H). LC-MS (ESI): m/z 715.4 [M+H]⁺.

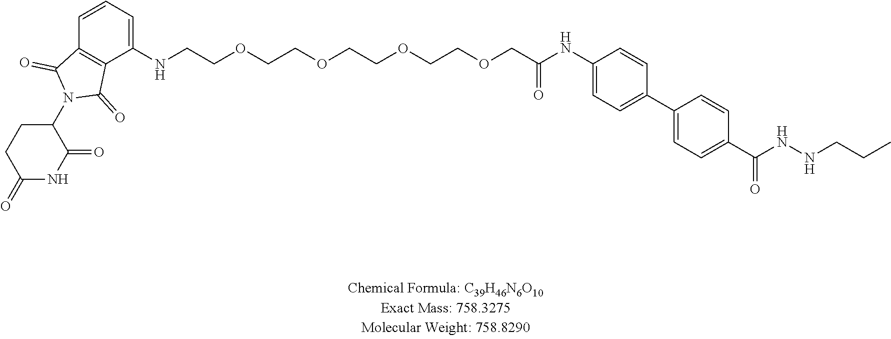 [SMILES: CCCNNC(=O)c1ccc(-c2ccc(NC(=O)COCCOCCOCCOCCNc3cccc4c3C(=O)N(C3CCC(=O)NC3=O)C4=O)cc2)cc1]

### 14-((2-(2,6-dioxopiperidin-3-yl)-1,3-dioxoisoindolin-4-yl)amino)-N-(4′-(2-propylhydrazine-1-carbonyl)-[1,1′-biphenyl]-4-yl)-3,6,9,12-tetraoxatetradecanamide (7c)

Following the general method above, compound 7c was obtained from S3 and 13 (6.8 mg, yield 33%). ¹H NMR (600 MHz, CD₃OD) δ 7.94 (d, J=8.5 Hz, 2H), 7.81-7.76 (m, 4H), 7.68 (d, J=8.7 Hz, 2H), 7.49 (dd, J=8.6, 7.1 Hz, 1H), 7.03-6.97 (m, 2H), 5.03 (dd, J=12.8, 5.5 Hz, 1H), 4.16 (s, 2H), 3.81-3.61 (m, 14H), 3.43 (t, J=5.3 Hz, 2H), 3.32-3.29 (m, 2H), 2.89-2.64 (m, 3H), 2.12-2.04 (m, 1H), 1.87-1.79 (m, 2H), 1.10 (t, J=7.5 Hz, 3H). LC-MS (ESI): m/z 759.4 [M+H]⁺.

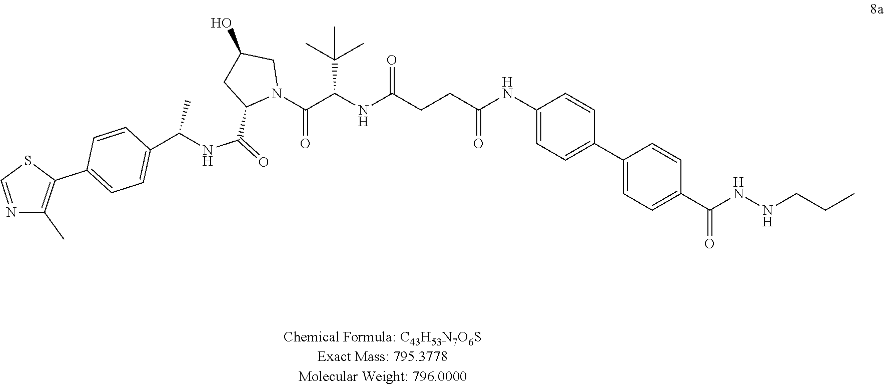 [SMILES: CCCNNC(=O)c1ccc(-c2ccc(NC(=O)CCC(=O)N[C@H](C(=O)N3C[C@H](O)C[C@H]3C(=O)N[C@@H](C)c3ccc(-c4scnc4C)cc3)C(C)(C)C)cc2)cc1]

N¹—((S)-1-((2S,4R)-4-hydroxy-2-(((S)-1-(4-(4-methylthiazol-5-yl)phenyl)ethyl)carbamoyl)pyrrolidine-1-yl)-3,3-dimethyl-1-oxobutan-2-yl)-N⁴-(4′-(2-propylhydrazine-1-carbonyl)-[1,1′-biphenyl]-4-yl)succinimide (8a)

Following the general method above, compound 8a was obtained from S4 and 13 (6.1 mg, yield 28%). ¹H NMR (600 MHz, CD₃OD) δ 8.89 (s, 1H), 8.56 (d, J=7.5 Hz, 1H), 8.00 (d, J=8.9 Hz, 1H), 7.91-7.81 (m, 2H), 7.77-7.61 (m, 7H), 7.48-7.41 (m, 4H), 5.07-5.00 (m, 1H), 4.66-4.58 (m, 2H), 4.48-4.42 (m, 1H), 3.94-3.86 (m, 1H), 3.77 (dd, J=11.1, 4.0 Hz, 1H), 3.01

-2.83 (m, 2H), 2.76-2.63 (m, 4H), 2.49 (s, 3H), 2.25-2.17 (m, 1H), 2.01-1.94 (m, 1H), 1.67-1.57 (m, 2H), 1.53 (d, J=7.0 Hz, 3H), 1.07 (s, 9H), 1.02 (t, J=7.2 Hz, 3H). LC-MS (ESI): m/z 796.4 [M+H]⁺.

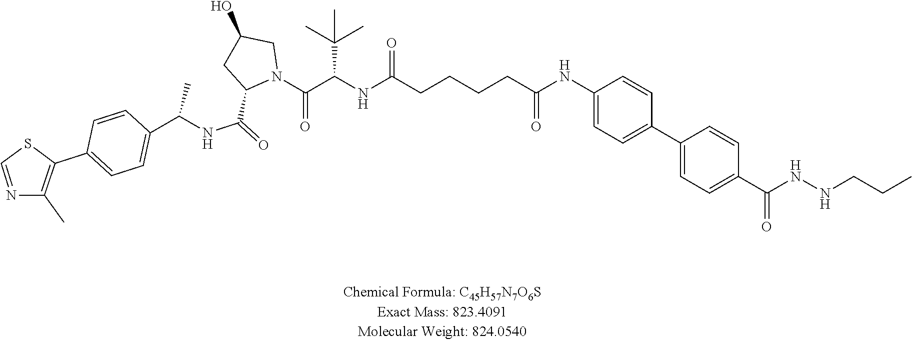 [SMILES: CCCNNC(=O)c1ccc(-c2ccc(NC(=O)CCCCC(=O)N[C@H](C(=O)N3C[C@H](O)C[C@H]3C(=O)N[C@@H](C)c3ccc(-c4scnc4C)cc3)C(C)(C)C)cc2)cc1]

### N¹—((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁶-(4′-(2-propylhydrazine-1-carbonyl)-[1,1′-biphenyl]-4-yl)adipamide (8b)

Following the general method above, compound 8b was obtained from S5 and 13 (3.8 mg, yield 17%). ¹H NMR (600 MHz, CD₃OD) δ 8.88 (s, 1H), 8.55 (d, J=7.5 Hz, 1H), 7.94-7.79 (m, 3H), 7.76-7.60 (m, 7H), 7.48-7.40 (m, 4H), 5.05-4.98 (m, 1H), 4.64 (d, J=9.0 Hz, 1H), 4.62-4.57 (m, 1H), 4.48-4.42 (m, 1H), 3.94-3.89 (m, 1H), 3.77 (dd, J=11.0, 4.0 Hz, 1H), 2.93-2.87 (m, 2H), 2.50-2.35 (m, 7H), 2.23-2.17 (m, 1H), 2.00-1.94 (m, 1H), 1.79-1.59 (m, 6H), 1.50 (d, J=7.1 Hz, 3H), 1.09-1.02 (m, 12H). LC-MS (ESI): m/z 824.4 [M+H]⁺.

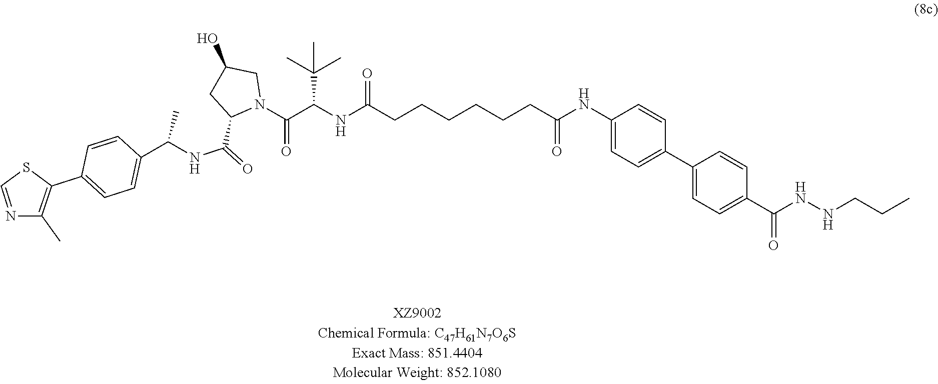 [SMILES: CCCNNC(=O)c1ccc(-c2ccc(NC(=O)CCCCCCC(=O)N[C@H](C(=O)N3C[C@H](O)C[C@H]3C(=O)N[C@@H](C)c3ccc(-c4scnc4C)cc3)C(C)(C)C)cc2)cc1]

N¹—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁵-(4′-(2-propylhydrazine-1-carbonyl)-[1,1′-biphenyl]-4-yl)octanediamide (8c, XZ9002)

Following the general method above, compound XZ9002 was obtained from 14 and 13 (3.6 mg, yield 16%). ¹H NMR (600 MHz, CD₃OD) δ 8.88 (s, 1H), 7.90-7.81 (m, 3H), 7.76-7.64 (m, 6H), 7.46-7.41 (m, 4H), 5.04-4.97 (m, 1H), 4.68-4.60 (m, 2H), 4.48-4.43 (m, 1H), 3.95-3.88 (m, 1H), 3.77 (dd, J=11.0, 4.0 Hz, 1H), 2.90 (t, J=7.3 Hz, 2H), 2.49 (s, 3H), 2.45-2.40 (m, 2H), 2.37-2.27 (m, 2H), 2.25-2.19 (m, 1H), 2.00-1.93 (m, 1H), 1.79-1.71 (m, 2H), 1.69-1.59 (m, 4H), 1.50 (d, J=7.0 Hz, 3H), 1.47-1.38 (m, 4H), 1.08-1.02 (m, 12H). LC-MS (ESI): m/z 852.6 [M+H]⁺.

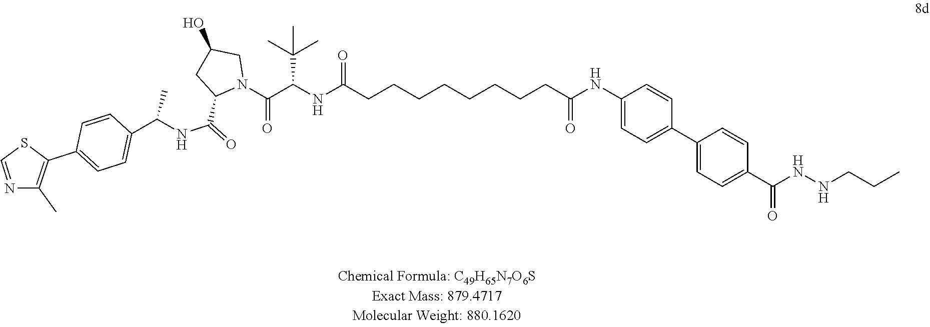 [SMILES: CCCNNC(=O)c1ccc(-c2ccc(NC(=O)CCCCCCCCC(=O)N[C@H](C(=O)N3C[C@H](O)C[C@H]3C(=O)N[C@@H](C)c3ccc(-c4scnc4C)cc3)C(C)(C)C)cc2)cc1]

### N¹—((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹⁰-(4′-(2-propylhydrazine-1-carbonyl)-[1,1′-biphenyl]-4-yl)decanediamide (8d)

Following the general method above, compound 8d was obtained from S6 and 13 (2.9 mg, yield 12%). ¹H NMR (600 MHz, CD₃OD) δ 8.89 (s, 1H), 7.91-7.86 (m, 2H), 7.82 (d, J=9.0 Hz, 1H), 7.75-7.64 (m, 6H), 7.47-7.41 (m, 4H), 5.06-4.98 (m, 1H), 4.67-4.62 (m, 1H), 4.60-4.58 (m, 1H), 4.47-4.42 (m, 1H), 3.94-3.84 (m, 1H), 3.76 (dd, J=11.0, 4.0 Hz, 1H), 2.90 (t, J=7.3 Hz, 2H), 2.49 (s, 3H), 2.44-2.40 (m, 2H), 2.34-2.24 (m, 2H), 2.23-2.18 (m, 1H), 2.00-1.93 (m, 1H), 1.78-1.71 (m, 2H), 1.65-1.57 (m, 4H), 1.52 (d, J=7.1 Hz, 3H), 1.43-1.34 (m, 8H), 1.07-1.02 (m, 12H). LC-MS (ESI): m/z 880.6 [M+H]⁺.

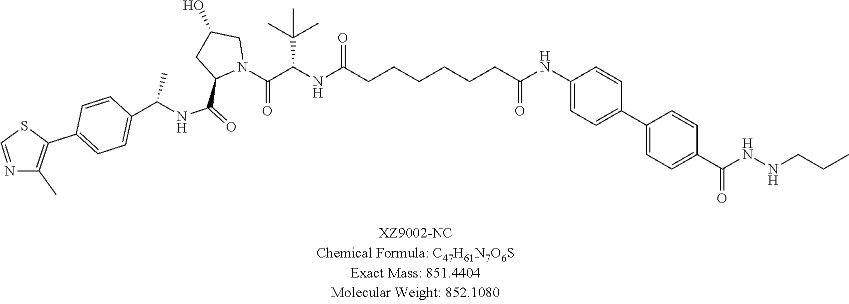 [SMILES: CCCNNC(=O)c1ccc(-c2ccc(NC(=O)CCCCCCC(=O)N[C@H](C(=O)N3C[C@@H](O)C[C@@H]3C(=O)N[C@@H](C)c3ccc(-c4scnc4C)cc3)C(C)(C)C)cc2)cc1]

### N¹—((S)-1-((2R,4S)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁵-(4′-(2-propylhydrazine-1-carbonyl)-[1,1′-biphenyl]-4-yl)octanediamide (XZ9002-NC)

Starting with S7 and S8, compound S9 was synthesized according to a previously published method. Following the general method, compound XZ9002-NC was obtained from S9 and 13 (3.2 mg, yield 16%). ¹H NMR (600 MHz, CD₃OD) δ 8.90 (s, 1H), 7.99-7.89 (m, 3H), 7.83-7.78 (m, 2H), 7.72-7.65 (m, 4H), 7.51-7.46 (m, 2H), 7.46-7.41 (m, 2H), 5.05-4.98 (m, 1H), 4.59-4.54 (m, 1H), 4.54-4.49 (m, 1H), 4.49-4.44 (m, 1H), 3.97 (dd, J=10.8, 5.0 Hz, 1H), 3.70 (dd, J=10.7, 3.1 Hz, 1H), 3.29-3.23 (m, 2H), 2.49 (s, 3H), 2.39-2.20 (m, 5H), 2.16-2.09 (m, 1H), 1.85-1.77 (m, 2H), 1.71-1.59 (m, 4H), 1.45 (d, J=7.0 Hz, 3H), 1.41-1.35 (m, 4H), 1.11-1.05 (m, 12H). LC-MS (ESI): m/z 852.5 [M+H]⁺.

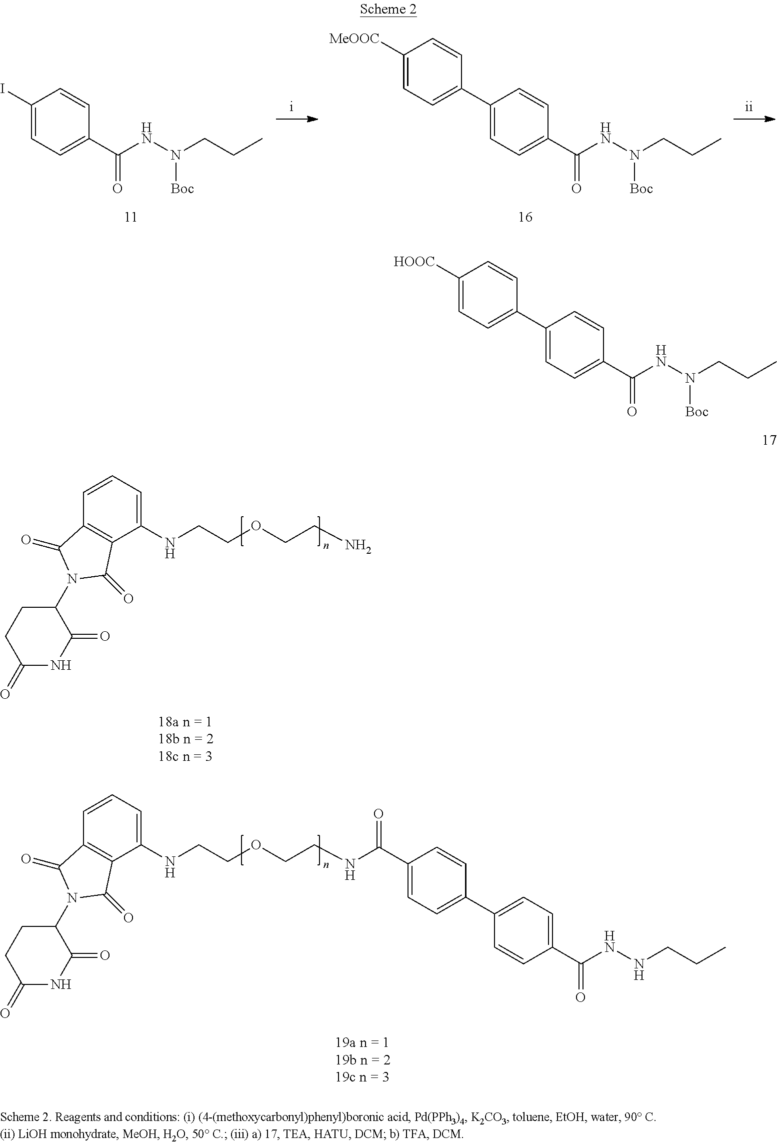

### Tert-Butyl 2-(4′-(methoxycarbonyl)[1,1′-biphenyl]-4-carbonyl)-1-propylhydrazine-1-carboxylate (16)

A mixture of compound 11 (300 mg, 0.74 mmol), (4-(methoxycarbonyl)phenyl)boronic acid (450 mg, 2.50 mmol), Pd(PPh₃)₄ (40 mg, 0.03 mmol), and K₂CO₃ (204 mg, 1.48 mmol) was stirred in toluene-EtOH-water (9:1:1, v/v/v, 2 mL) at 90° C. under an argon atmosphere for 16 h. The reaction mixture was cooled to room temperature and poured into water. The resulting solution was extracted with ethyl acetate. The organic phase was washed with water x 1, brine x 1, dried over Na₂SO₄, filtered, and evaporated to dryness. The residue was further purified by column chromatography to afford the title compound (290 mg, yield 95%). ¹H NMR (600 MHz, CDCl₃) δ 8.15 (d, J=8.1 Hz, 2H), 7.93-7.86 (m, 2H), 7.85-7.57 (m, 4H), 3.97 (s, 3H), 3.60 (t, J=7.2 Hz, 2H), 1.71-1.64 (m, 2H), 1.59-1.38 (m, 9H), 0.98 (t, J=7.4 Hz, 3H). LC-MS (ESI): m/z 413.2 [M+H]⁺.

### 4′-(2-(tert-butoxycarbonyl)-2-propylhydrazine-1-carbonyl)[1,1′-biphenyl]-4-carboxylic Acid (17)

A mixture of 16 (120 mg, 0.29 mmol) and LiOH monohydrate (75 mg, 1.79 mmol) was stirred in MeOH-water (4/1, v/v, 3 mL) at 50° C. for 2 h. The reaction mixture was cooled to room temperature and poured into water. The resulting solution was extracted with ethyl acetate. The organic phase was washed with water x 1, brine x 1, dried over Na₂SO₄, filtered, and evaporated to dryness. The residue was used directly in the next step (88 mg, yield 76%). LC-MS (ESI): m/z 399.1 [M+H]⁺.

### General Method B for Compounds 19a-c

Amines 18a-c were prepared according to a previously published procedure. A mixture of corresponding acid (1.0 equiv.), amine (1.0 equiv.), HATU (1.05 equiv.) and Et₃N (5.0 equiv.) in DCM was stirred at room temperature for 1 h. The mixture was poured into water and extracted with DCM. The combined organic layers were washed with NH₄Cl (aq.)×1, brine x 1, dried over anhydrous Na₂SO₄, filtered, and concentrated under vacuum. The residue was dissolved in DCM and treated with TFA (20 equiv.). The mixture was stirred at room temperature for 4 h and concentrated under reduced pressure. The crude product was purified by flash column chromatography to afford the desired compound.

### N-(2-(2-((2-(2,6-dioxopiperidin-3-yl)-1,3-dioxoisoindolin-4-yl)amino)ethoxy)ethyl)-4′-(2-propylhydrazine-1-carbonyl)-[1,1′-biphenyl]-4-carboxamide (19a)

Following the general method B above, compound 19a was obtained from 18a and 17 (22.2 mg, yield 92%). ¹H NMR (600 MHz, CD₃OD) δ 8.02-7.96 (m, 2H), 7.91-7.82 (m, 4H), 7.78-7.73 (m, 2H), 7.51 (dd, J=8.6, 7.1 Hz, 1H), 7.11 (d, J=8.6 Hz, 1H), 6.98 (d, J=7.0 Hz, 1H), 4.96 (dd, J=12.6, 5.5 Hz, 1H), 3.86-3.70 (m, 4H), 3.69-3.59 (m, 2H), 3.58-3.52 (m, 2H), 3.25-3.16 (m, 2H), 2.79-2.57 (m, 3H), 2.07-1.96 (m, 1H), 1.84-1.72 (m, 2H), 1.08 (t, J=7.5 Hz, 3H). LC-MS (ESI): m/z 641.3 [M+H]⁺.

### N-(2-(2-(2-((2-(2,6-dioxopiperidin-3-yl)-1,3-dioxoisoindolin-4-yl)amino)ethoxy)ethoxy)ethyl)-4′-(2-propylhydrazine-1-carbonyl)-[1,1′-biphenyl]-4-carboxamide (19b)

Following the general method B above, compound 19b was obtained from 18b and 17 (12.2 mg, yield 47%). ¹H NMR (600 MHz, CD₃OD) δ 7.95 (d, J=8.5 Hz, 2H), 7.91 (d, J=8.5 Hz, 2H), 7.78 (d, J=8.4 Hz, 2H), 7.70 (d, J=8.4 Hz, 2H), 7.46 (dd, J=8.6, 7.1 Hz, 1H), 7.01 (d, J=8.5 Hz, 1H), 6.96 (d, J=7.0 Hz, 1H), 5.02 (dd, J=12.7, 5.5 Hz, 1H), 3.82-3.68 (m, 8H), 3.66-3.58 (m, 2H), 3.45 (t, J=5.2 Hz, 2H), 3.15 (t, J=7.6 Hz, 2H), 2.91-2.60 (m, 3H), 2.11-2.02 (m, 1H), 1.80-1.70 (m, 2H), 1.07 (t, J=7.4 Hz, 3H). LC-MS (ESI): m/z 685.2 [M+H]⁺.

### N-(2-(2-(2-(2-((2-(2,6-dioxopiperidin-3-yl)-1,3-dioxoisoindolin-4-yl)amino)ethoxy)ethoxy)ethoxy)ethyl)-4′-(2-propylhydrazine-1-carbonyl)-[1,1′-biphenyl]-4-carboxamide (19c)

Following the general method B above, compound 19c was obtained from 18c and 17 (17.8 mg, yield 65%). ¹H NMR (600 MHz, CD₃OD) δ 7.98-7.92 (m, 4H), 7.80 (d, J=8.5 Hz, 2H), 7.77 (d, J=8.4 Hz, 2H), 7.49 (dd, J=8.5, 7.1 Hz, 1H), 7.04-6.97 (m, 2H), 5.04 (dd, J=12.8, 5.5 Hz, 1H), 3.74-3.59 (m, 14H), 3.42 (t, J=5.3 Hz, 2H), 3.25-3.18 (m, 2H), 2.91-2.64 (m, 3H), 2.13-2.05 (m, 1H), 1.82-1.73 (m, 2H), 1.08 (t, J=7.4 Hz, 3H). LC-MS (ESI): m/z 729.4 [M+H]⁺.

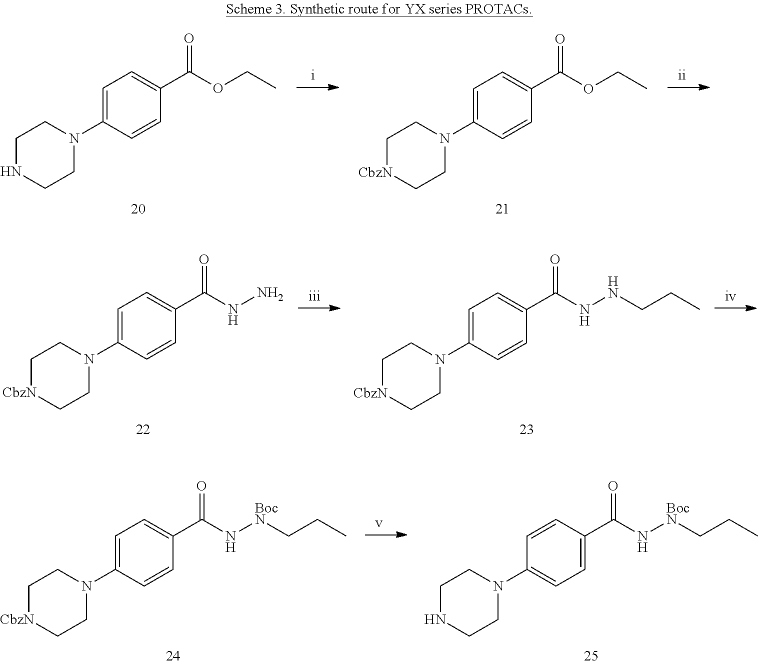 [SMILES: CCOC(=O)c1ccc(N2CCNCC2)cc1 ; CCOC(=O)c1ccc(N2CCN(C(=O)OCc3ccccc3)CC2)cc1 ; NNC(=O)c1ccc(N2CCN(C(=O)OCc3ccccc3)CC2)cc1 ; CCCNNC(=O)c1ccc(N2CCN(C(=O)OCc3ccccc3)CC2)cc1 ; CCCN(NC(=O)c1ccc(N2CCN(C(=O)OCc3ccccc3)CC2)cc1)C(=O)OC(C)(C)C ; CCCN(NC(=O)c1ccc(N2CCNCC2)cc1)C(=O)OC(C)(C)C]

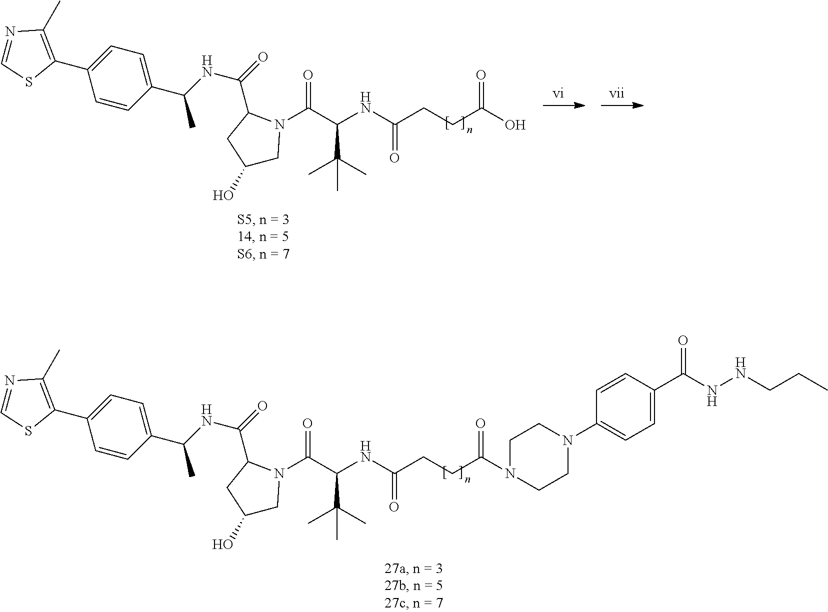

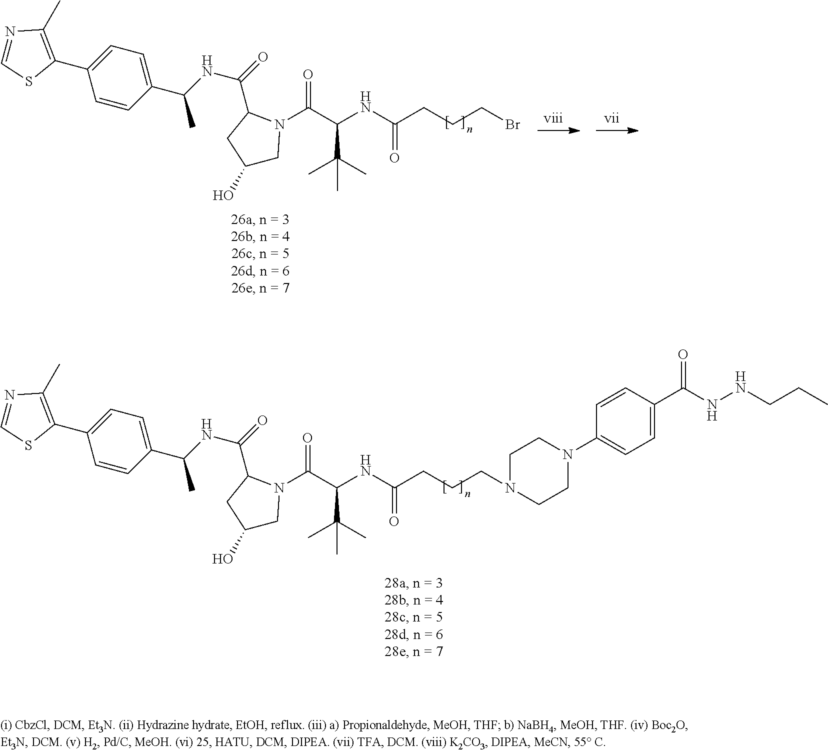

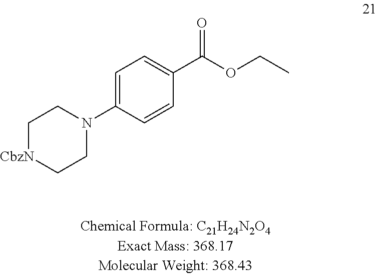 [SMILES: CCOC(=O)c1ccc(N2CCN(C(=O)OCc3ccccc3)CC2)cc1]

A mixture of 20 (2.0 g, 8.55 mmol), CbzCl (1.67 g, 9.83 mmol), and Et₃N (3.5 mL) in DCM (30 mL) was stirred at room temperature for 12 h. The mixture was poured into water and extracted with EA. The combined organic layers were washed with NaHCO₃(aq.)×1, brine x 1, dried over anhydrous Na₂SO₄, filtered, and concentrated under vacuum. The crude product was purified by flash column chromatography to afford the title compound (2.74 g, yield 87%). ¹H NMR (600 MHz, Chloroform-d) δ 7.97-7.92 (m, 2H), 7.40-7.31 (m, 5H), 6.89-6.84 (m, 2H), 5.17 (s, 2H), 4.33 (q, J=7.1 Hz, 2H), 3.71-3.62 (m, 4H), 3.31 (s, 4H), 1.37 (t, J=7.1 Hz, 3H). LC-MS (ESI): m/z 369.2 [M+H]⁺.

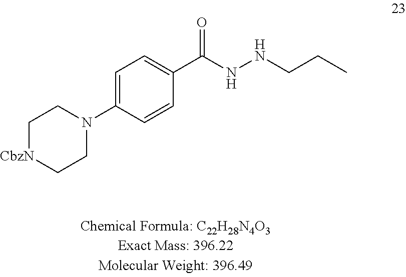 [SMILES: CCCNNC(=O)c1ccc(N2CCN(C(=O)OCc3ccccc3)CC2)cc1]

A mixture of 21 (2.0 g, 5.4 mmol), Hydrazine monohydrate (2.7 g, 54 mmol) in ethanol (30 mL) was refluxed for 24 h. The mixture was cooled to room temperature and concentrated under vacuum, the resulting crude 22 was used without further purification. A mixture of 22 (2.0 g, 8.5 mmol), Propionaldehyde (990 mg, 17.1 mmol) in MeOH-THF (1:1, v/v, 30 mL) was stirred at room temperature for 2 h. The solvent was removed under reduced pressure and the residue was dissolved in MeOH-THF (1:1, v/v, 30 mL). The solution was treated with NaBH₄ (965 mg, 25.5 mmol) and stirred for 30 min. Then it was diluted with water and extracted with ethyl acetate. The organic phase was washed with water x 1, brine×1, dried over Na₂SO₄, filtered, and evaporated to dryness. The crude product was purified by flash column chromatography to afford the title compound (1.7 g, yield 51%). LC-MS (ESI): m/z 397.2 [M+H]⁺.

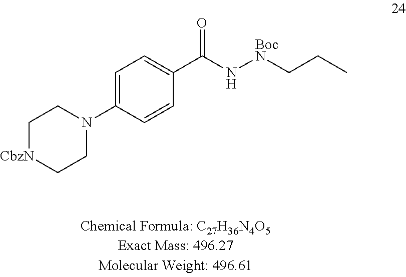 [SMILES: CCCN(NC(=O)c1ccc(N2CCN(C(=O)OCc3ccccc3)CC2)cc1)C(=O)OC(C)(C)C]

A mixture of 23 (1.6 g, 4.04 mmol), Boc₂O (969 mg, 4.45 mmol), and Et₃N (1.3 mL) in DCM (30 mL) was stirred at room temperature for 12 h. The mixture was poured into water and extracted with EA. The combined organic layers were washed with brine x 1, dried over anhydrous Na₂SO₄, filtered, and concentrated under vacuum. The crude product was purified by flash column chromatography to afford the title compound (1.64 g, yield 83%). ¹H NMR (600 MHz, Chloroform-d) δ 7.76-7.68 (m, 2H), 7.41-7.31 (m, 5H), 6.93-6.77 (m, 2H), 5.17 (s, 2H), 3.73-3.62 (m, 4H), 3.54 (t, J=7.3 Hz, 2H), 3.27 (s, 4H), 1.65-1.34 (m, 11H), 0.92 (t, J=7.4 Hz, 3H). LC-MS (ESI): m/z 497.2 [M+H]⁺.

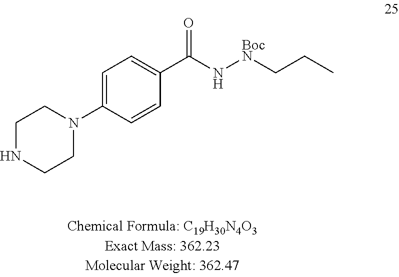 [SMILES: CCCN(NC(=O)c1ccc(N2CCNCC2)cc1)C(=O)OC(C)(C)C]

A mixture of 24 (1.6 g, 3.25 mmol), Pd/C (35 mg) in MeOH (30 mL) was purged by nitrogen then hydrogen and stirred at room temperature for 12 h. The mixture was filtered and concentrated under vacuum. The crude product was used without further purification. LC-MS (ESI): m/z 363.2 [M+H]⁺.

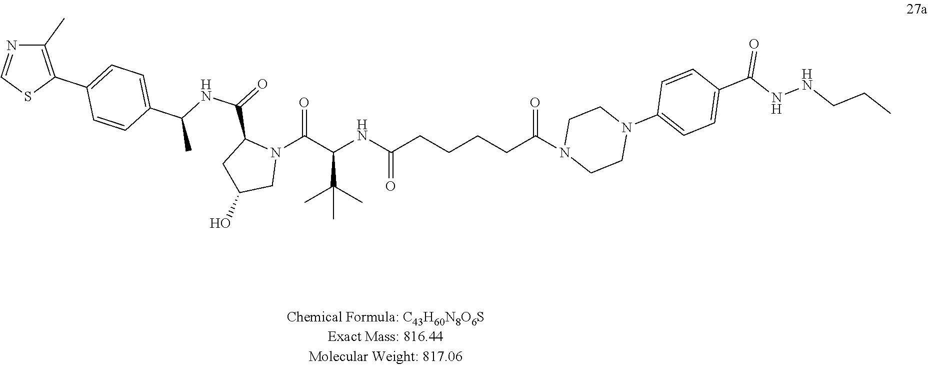 [SMILES: CCCNNC(=O)c1ccc(N2CCN(C(=O)CCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(6-oxo-6-(4-(4-(2-propylhydrazine-1-carbonyl)phenyl)piperazin-1-yl)hexanamido)butanoyl)-4-hydroxy-N—((S)-1-(4-(4-methylthiazol-5-yl)phenyl)ethyl)pyrrolidine-2-carboxamide (27a)

Following the general method A above, compound 27a was obtained from S5 and 25 (12.1 mg, yield 59%). ¹H NMR (600 MHz, Chloroform-d) δ 8.67 (s, 1H), 7.72 (s, 1H), 7.69-7.64 (m, 2H), 7.48 (d, J=7.9 Hz, 1H), 7.42-7.34 (m, 4H), 6.88-6.84 (m, 2H), 6.50 (d, J=8.7 Hz, 1H), 5.13-5.06 (m, 1H), 4.74 (t, J=8.0 Hz, 1H), 4.58 (d, J=8.7 Hz, 1H), 4.52-4.47 (m, 1H), 4.10 (d, J=10.9 Hz, 1H), 3.74 (q, J=4.7 Hz, 2H), 3.64-3.57 (m, 3H), 3.31-3.22 (m, 4H), 2.89 (t, J=7.3 Hz, 2H), 2.56-2.48 (m, 4H), 2.39-2.19 (m, 4H), 2.10-2.05 (m, 1H), 1.70-1.54 (m, 6H), 1.47 (d, J=6.9 Hz, 3H), 1.05 (s, 9H), 0.97 (t, J=7.4 Hz, 3H).). LC-MS (ESI): m/z 817.5 [M+H]⁺.

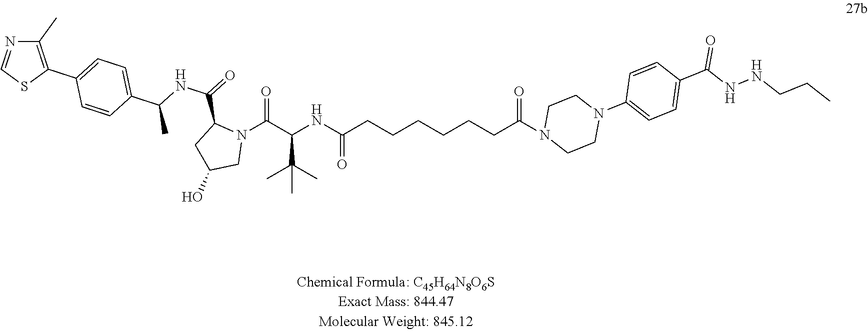 [SMILES: CCCNNC(=O)c1ccc(N2CCN(C(=O)CCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(8-oxo-8-(4-(4-(2-propylhydrazine-1-carbonyl)phenyl)piperazin-1-yl)octanamido)butanoyl)-4-hydroxy-N—((S)-1-(4-(4-methylthiazol-5-yl)phenyl)ethyl)pyrrolidine-2-carboxamide (27b)

Following the general method A above, compound 27b was obtained from 14 and 25 (11.0 mg, yield 52%). ¹H NMR (600 MHz, Chloroform-d) δ 8.68 (s, 1H), 7.69 (t, J=8.3 Hz, 3H), 7.49 (d, J=7.9 Hz, 1H), 7.39 (dd, J=20.1, 8.3 Hz, 4H), 6.87 (d, J=8.9 Hz, 2H), 6.25 (d, J=8.8 Hz, 1H), 5.12-5.06 (m, 1H), 4.73 (t, J=8.0 Hz, 1H), 4.58 (d, J=8.9 Hz, 1H), 4.52-4.47 (m, 1H), 4.10 (d, J=11.4 Hz, 1H), 3.80-3.72 (m, 2H), 3.64-3.56 (m, 3H), 3.32-3.22 (m, 4H), 2.89 (t, J=7.3 Hz, 2H), 2.57-2.46 (m, 4H), 2.38-2.30 (m, 2H), 2.28-2.16 (m, 2H), 2.12-2.05 (m, 1H), 1.67-1.52 (m, 6H), 1.48 (d, J=6.9 Hz, 3H), 1.39-1.29 (m, 4H), 1.04 (s, 9H), 0.97 (t, J=7.4 Hz, 3H). LC-MS (ESI): m/z 845.2 [M+H]⁺.

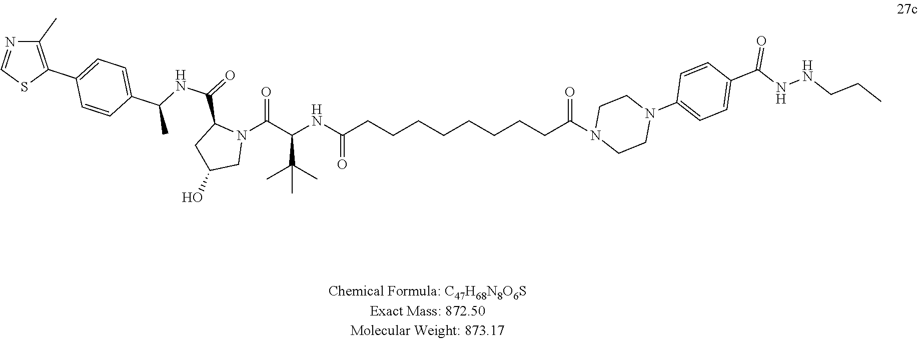 [SMILES: CCCNNC(=O)c1ccc(N2CCN(C(=O)CCCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(10-oxo-10-(4-(4-(2-propylhydrazine-1-carbonyl)phenyl)piperazin-1-yl)decanamido)butanoyl)-4-hydroxy-N—((S)-1-(4-(4-methylthiazol-5-yl)phenyl)ethyl)pyrrolidine-2-carboxamide (27c)

Following the general method A above, compound 27c was obtained from S6 and 25 (3.8 mg, yield 27%). ¹H NMR (600 MHz, Chloroform-d) δ 8.67 (s, 1H), 7.74-7.55 (m, 3H), 7.52-7.30 (m, 5H), 6.88 (d, J=8.8 Hz, 2H), 6.14 (d, J=8.7 Hz, 1H), 5.12-5.06 (m, 1H), 4.74 (t, J=7.9 Hz, 1H), 4.58-4.49 (m, 2H), 4.13 (d, J=11.5 Hz, 1H), 3.80-3.71 (m, 2H), 3.68-3.55 (m, 3H), 3.36-3.20 (m, 4H), 2.94-2.83 (m, 2H), 2.61-2.42 (m, 4H), 2.40-2.32 (m, 2H), 2.25-2.14 (m, 2H), 2.11-2.05 (m, 1H), 1.80-1.51 (m, 6H), 1.48 (d, J=6.9 Hz, 3H), 1.39-1.21 (m, 8H), 1.04 (s, 9H), 0.97 (t, J=7.4 Hz, 3H). LC-MS (ESI): m/z 873.5 [M+H]⁺.

### General Method C for 28a, 28b, 28c, 28d and 28e

26a-d were synthesized according to method B. A mixture of 21 (1 equivalent), 26a-d (1.0 equiv.), KI (0.1 equiv.), DI PEA (3.0 equiv.) and K₂CO₃ (3.0 equiv.) in MeCN was stirred at 55° C. for 24 h. The mixture was poured into water and extracted with EA. The combined organic layers were washed with brine x 1, dried over anhydrous Na₂SO₄, filtered, and concentrated under vacuum. The crude product was purified by flash column chromatography. The product was dissolved in DCM and treated with TFA (20 equiv.). The mixture was stirred at room temperature for 4 h and concentrated under reduced pressure. EA was added. The combined organic layers were washed with NaHCO₃(aq.)×1, brine×1, and dried over anhydrous Na₂SO₄, filtered, and concentrated under vacuum. The crude product was purified by flash column chromatography to afford the title compound.

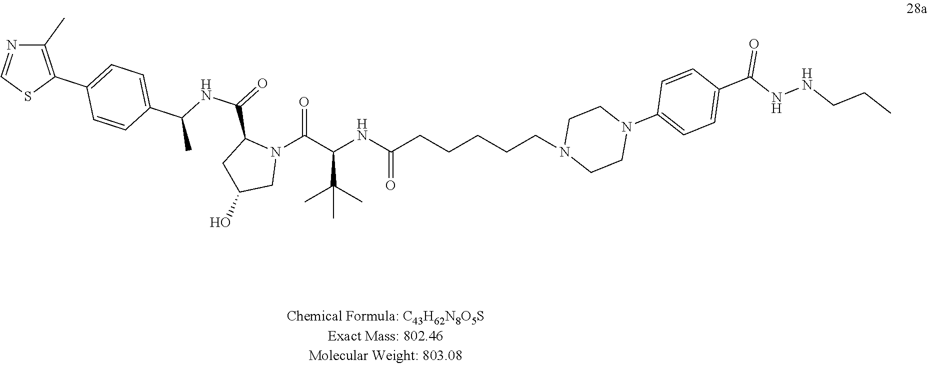 [SMILES: CCCNNC(=O)c1ccc(N2CCN(CCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(6-(4-(4-(2-propylhydrazine-1-carbonyl)phenyl)piperazin-1-yl)hexanamido)butanoyl)-4-hydroxy-N—((S)-1-(4-(4-methylthiazol-5-yl)phenyl)ethyl)pyrrolidine-2-carboxamide (28a)

Following the general method C above, compound 28a was obtained from 26a and 25 (8.1 mg, yield 37%). ¹H NMR (600 MHz, Chloroform-d) δ 8.68 (s, 1H), 7.69-7.57 (m, 3H), 7.44-7.34 (m, 5H), 6.90-6.85 (m, 2H), 6.17 (d, J=8.7 Hz, 1H), 5.12-5.05 (m, 1H), 4.73 (t, J=7.9 Hz, 1H), 4.57 (d, J=8.8 Hz, 1H), 4.53-4.48 (m, 1H), 4.12-4.08 (m, 1H), 3.59 (dd, J=11.4, 3.6 Hz, 1H), 3.29 (t, J=5.2 Hz, 4H), 2.89 (t, J=7.3 Hz, 2H), 2.62-2.50 (m, 8H), 2.41-2.35 (m, 2H), 2.28-2.18 (m, 2H), 2.09-2.03 (m, 1H), 1.69-1.45 (m, 9H), 1.39-1.31 (m, 2H), 1.05 (s, 9H), 0.97 (t, J=7.4 Hz, 3H). LC-MS (ESI): m/z 803.5 [M+H]⁺.

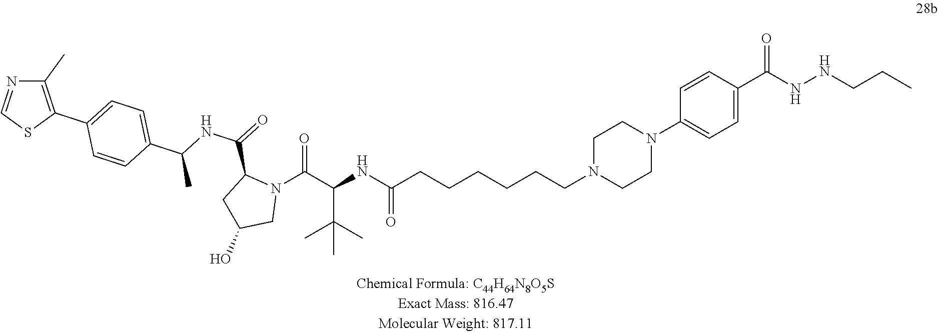 [SMILES: CCCNNC(=O)c1ccc(N2CCN(CCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(7-(4-(4-(2-propylhydrazine-1-carbonyl)phenyl)piperazin-1-yl)heptanamido)butanoyl)-4-hydroxy-N—((S)-1-(4-(4-methylthiazol-5-yl)phenyl)ethyl)pyrrolidine-2-carboxamide (28b)

Following the general method above, compound 28b was obtained from 26b and 25 (15.0 mg, yield 45%). ¹H NMR (600 MHz, Chloroform-d) δ 8.68 (s, 1H), 7.68-7.58 (m, 3H), 7.44-7.36 (m, 5H), 6.91-6.85 (m, 2H), 6.11 (d, J=8.7 Hz, 1H), 5.12-5.06 (m, 1H), 4.74 (t, J=7.9 Hz, 1H), 4.58-4.50 (m, 2H), 4.18-4.11 (m, 1H), 3.59 (dd, J=11.4, 3.7 Hz, 1H), 3.30 (t, J=5.2 Hz, 4H), 2.92-2.86 (m, 2H), 2.64-2.51 (m, 8H), 2.38 (t, J=7.7 Hz, 2H), 2.24-2.20 (m, 2H), 2.10-2.04 (m, 1H), 1.66-1.46 (m, 9H), 1.38-1.31 (m, 4H), 1.05 (s, 9H), 0.97 (t, J=7.4 Hz, 3H). LC-MS (ESI): m/z 817.5 [M+H]⁺.

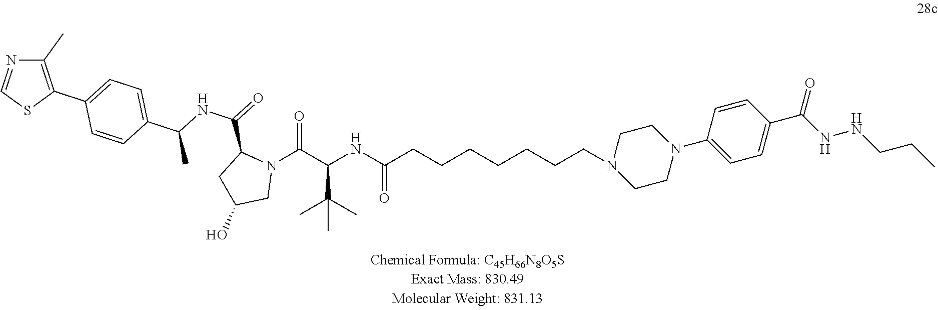 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(8-(4-(4-(2-propylhydrazine-1-carbonyl)phenyl)piperazin yl)octanamido)butanoyl)-4-hydroxy-N—((S)-1-(4-(4-methylthiazol-5-yl)phenyl)ethyl)pyrrolidine carboxamide (28c)

Following the general method above, compound 28c was obtained from 26c and 25 (10.2 mg, yield 45%). ¹H NMR (600 MHz, Chloroform-d) δ 8.68 (s, 1H), 7.68-7.63 (m, 2H), 7.56 (s, 1H), 7.45-7.34 (m, 5H), 6.92-6.84 (m, 2H), 6.12 (d, J=8.7 Hz, 1H), 5.11-5.05 (m, 1H), 4.73 (t, J=7.9 Hz, 1H), 4.57 (d, J=8.7 Hz, 1H), 4.54-4.50 (m, 1H), 4.11 (dt, J=11.4, 2.0 Hz, 1H), 3.58 (dd, J=11.4, 3.6 Hz, 1H), 3.35-3.26 (m, 4H), 2.89 (t, J=7.3 Hz, 2H), 2.65-2.49 (m, 8H), 2.42-2.35 (m, 2H), 2.25-2.18 (m, 2H), 2.08-2.03 (m, 1H), 1.66-1.45 (m, 9H), 1.35-1.27 (m, 6H), 1.05 (s, 9H), 0.97 (t, J=7.4 Hz, 3H). LC-MS (ESI): m/z 831.5 [M+H]⁺.

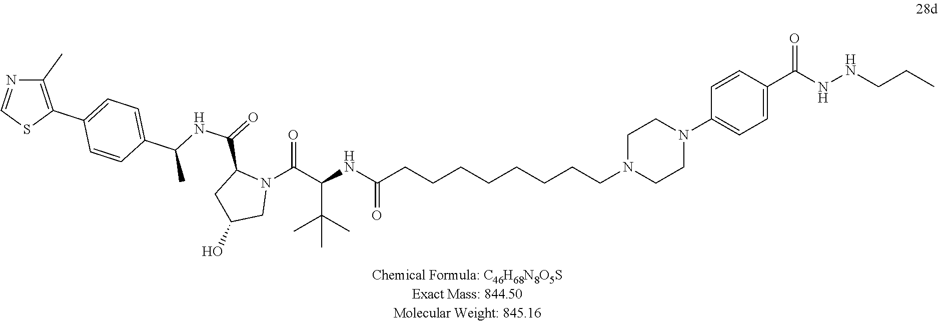 [SMILES: CCCNNC(=O)c1ccc(N2CCN(CCCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(9-(4-(4-(2-propylhydrazine-1-carbonyl)phenyl)piperazin-1-yl)nonanamido)butanoyl)-4-hydroxy-N—((S)-1-(4-(4-methylthiazol-5-yl)phenyl)ethyl)pyrrolidine-2-carboxamide (28d)

Following the general method above, compound 28d was obtained from 26d and 25 (12.3 mg, yield 32%). ¹H NMR (600 MHz, Chloroform-d)¹H NMR (600 MHz, Chloroform-d) δ 8.68 (s, 1H), 7.72-7.62 (m, 3H), 7.45-7.36 (m, 5H), 6.92-6.86 (m, 2H), 6.15 (d, J=8.7 Hz, 1H), 5.12-5.06 (m, 1H), 4.73 (t, J=7.9 Hz, 1H), 4.56 (d, J=8.8 Hz, 1H), 4.51 (dt, J=4.1, 2.1 Hz, 1H), 4.12 (dt, J=11.4, 1.9 Hz, 1H), 3.59 (dd, J=11.3, 3.7 Hz, 1H), 3.31 (t, J=5.2 Hz, 4H), 2.89 (t, J=7.3 Hz, 2H), 2.63-2.53 (m, 8H), 2.39 (dd, J=6.1, 3.9 Hz, 2H), 2.23-2.19 (m, 2H), 2.09-2.05 (m, 1H), 1.64-1.47 (m, 9H), 1.34-1.26 (m, 8H), 1.04 (s, 9H), 0.97 (t, J=7.4 Hz, 3H). LC-MS (ESI): m/z 845.6 [M+H]⁺.

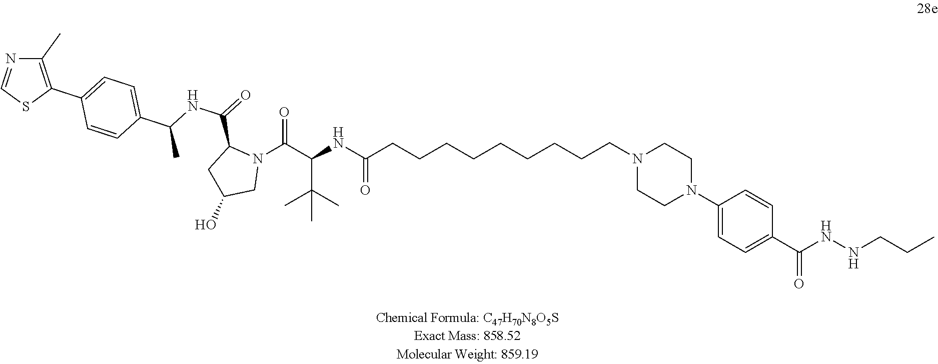 [SMILES: CCCNNC(=O)c1ccc(N2CCN(CCCCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### (2S,4R)-1-((S)-3,3-dimethyl-2-(10-(4-(4-(2-propylhydrazine-1-carbonyl)phenyl)piperazin-1-yl)decanamido)butanoyl)-4-hydroxy-N—((S)-1-(4-(4-methylthiazol-5-yl)phenyl)ethyl)pyrrolidine-2-carboxamide (28e)

Following the general method above, compound 28e was obtained from 26e and 25 (11.0 mg, yield 46%). ¹H NMR (600 MHz, Chloroform-d) δ 8.68 (s, 1H), 7.70-7.63 (m, 3H), 7.46-7.43 (m, 1H), 7.43-7.34 (m, 4H), 6.91-6.84 (m, 2H), 6.15 (d, J=8.7 Hz, 1H), 5.11-5.06 (m, 1H), 4.72 (t, J=7.9 Hz, 1H), 4.56 (d, J=8.8 Hz, 1H), 4.54-4.50 (m, 1H), 4.13-4.08 (m, 1H), 3.59 (dd, J=11.4, 3.7 Hz, 1H), 3.31 (t, J=5.1 Hz, 4H), 2.88 (t, J=7.3 Hz, 2H), 2.63-2.50 (m, 8H), 2.41-2.36 (m, 2H), 2.19 (t, J=7.6 Hz, 2H), 2.08-2.03 (m, 1H), 1.64-1.45 (m, 9H), 1.34-1.23 (m, 10H), 1.04 (s, 9H), 0.97 (t, J=7.4 Hz, 3H). LC-MS (ESI): m/z 859.6 [M+H]⁺.

### Example 2: Biological Methods

### In Vitro HDAC Activity Assay

Purified HDAC1, HDAC2, and HDAC3 (in complex with the deacetylase activation domain of the human NCOR2 (amino acids 395-498)) were obtained from Enzo Life Sciences (HDAC1, catalog # BML-SE456-0050) and BPS Bioscience (HDAC2, catalog #50002 and HDAC3, catalog #50003), respectively. The enzyme activities were tested using the HDAC-Glo I/II reagents (Promega) according to a protocol provided by the manufacturer. Select assays results are shown in Table 1.

### Cell Culture, Viability Assays, Clonogenic Growth Assays, and Western Blotting

Cell lines were obtained from ATCC and subsequently authenticated. Cells were cultured with DMEM supplemented with 10% bovine calf serum, penicillin to 10 units/mL, and streptomycin to 10 μg/mL. For viability assays, 5,000 cells/well were seeded in 96-well plates. Compounds at a specified concentration or DMSO were added 24 h later. Viability assays were performed 72 h after compound addition using the CellTiter-Glo reagents (Promega). For colony formation assay, cells (˜1,000 cells/well) were seeded in a 24-well plate in quadruplicates. At 24 h after seeding, DMSO, XZ9002, or XZ9002-NC were added at a specified concentration. Medium was replaced with freshly prepared medium containing DMSO, XZ9002 or XZ9002-NC every five days until visible colonies appeared, typically in 15 to 20 days after initial cell seeding. Cells were fixed with 4% paraformaldehyde for 15 min and stained with 2% methylene blue in 20% ethanol for 30 min to 1 h. The cells were washed twice with distilled water and dried in air. For western blotting, cell cultures were exposed to compounds as indicated in relevant figures. Medium was removed from culture plate, and 1 x passive lysis buffer was then added, the plates were frozen at −80° C. overnight and thawed at room temperature. The total cell lysates were mixed with a one fifth volume of 6x SDS sample buffer, heated at 95° C. for 5 min and cooled on ice. The lysates were cleared by centrifugation and then subjected to SDS-PAGE and western blotting essentially as described. Antibodies used for Western blotting include anti-HDAC3 (Abcam, Ab32369, 1:5,000 dilution), anti-HDAC1 (Sigma-Aldrich, H3284, 1:10,000 dilution), anti-HDAC2 (Santa Cruz Biotechnology, SC-7899, 1:10,000 dilution), anti-HDAC6 (Cell Signaling Technology, 7558, 1:1,000 dilution), anti-histone H3 acetylated at lysine 27 (H3K27ac, Cell Signaling Technology, 8173, 1:10,000 dilution), anti-histone H4 acetylated at lysine 5 (H4K5ac, Abcam, ab51997, 1:10,000 dilution), anti-acetylated lysine (Cell Signaling Technology, 9441, 1:10,000 dilution), anti-a-tubulin (Sigma-Aldrich, T5168, 1:20,000 dilution).

### Example 3: In Vitro Inhibitory Potencies of New PROTACs Against HDAC1-3

The HDAC binding affinities were measured using the HDAC-Glo™ I/II Assays.24 Compound 15 exhibited good inhibitory activities against tested HDAC isoforms with at least a 13-fold preference for HDAC3 (Table S1, ESI†), confirming the linker connection position is suitable. CRBN-based PROTACs 7a-c with PEG linkers maintained the in vitro HDAC inhibitory activities whereas VHL-based PROTACs 8a-d with alkane linkers exhibited compromised HDAC binding affinities. Besides, with the increase of carbon linker length in 8a-d series, the HDAC binding activities slightly decreased.

In vitro inhibitory potencies of the new PROTACs disclosed herein against HDAC1-3 are presented in Table 1:

TABLE 1
In vitro Inhibitory Potencies of PROTACs against HDAC1-3

| | IC₅₀ (μM)^{a} | | |
|---|---|---|---|
| Compound | HDAC3 | HDAC1 | HDAC2 |
| 15 | 0.013 ± 0.001 | 0.072 ± 0.010 | 0.082 ± 0.025 |
| 7a | 0.043 ± 0.018 | 0.082 ± 0.025 | 0.410 ± 0.014 |
| 7b | 0.038 ± 0.023 | 0.090 ± 0.014 | 0.400 ± 0.030 |
| 7c | 0.072 ± 0.015 | 0.125 ± 0.035 | 0.450 ± 0.070 |
| 8a | 0.14 ± 0.04 | 0.30 ± 0.07 | 1.40 ± 0.14 |
| 8b | 0.32 ± 0.03 | 0.36 ± 0.06 | 1.65 ± 0.07 |
| XZ9002 | 0.35 ± 0.07 | 0.65 ± 0.07 | 1.55 ± 0.07 |
| 8d | 0.55 ± 0.07 | 1.50 ± 0.14 | 1.42 ± 0.25 |

^{a}Each value is the average of three independent assays ± standard deviation.

### Example 4: Compound Selectivity for HDAC3

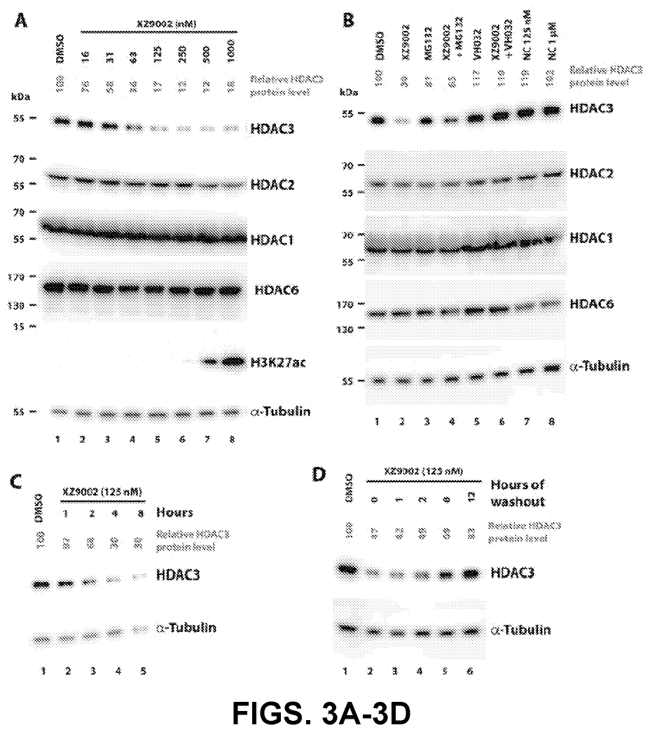

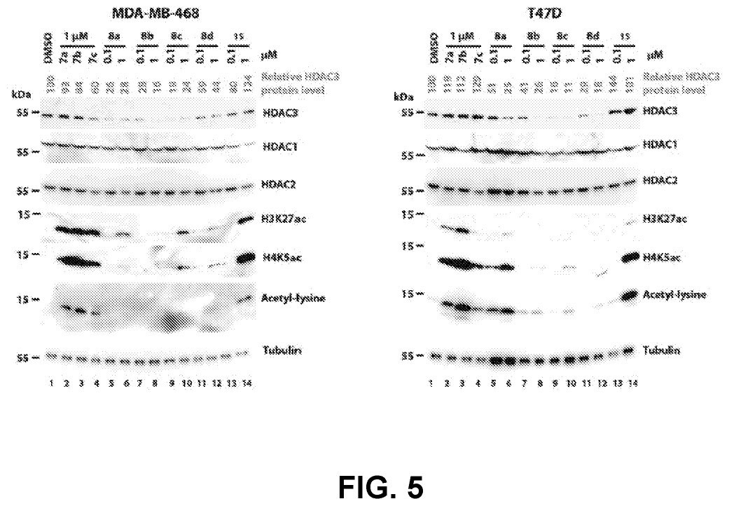

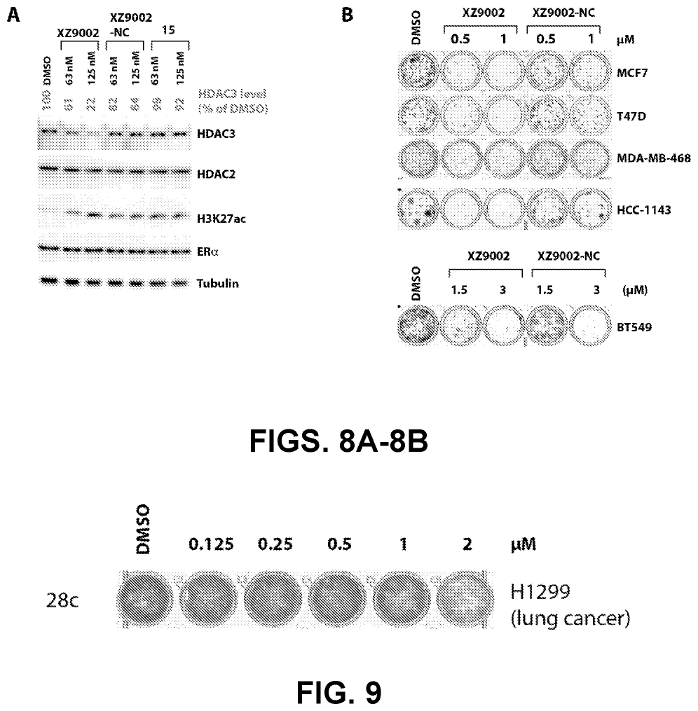

The HDAC degradation ability of all designed PROTACs was examined in triple-negative breast cancer cell line MDA-MB-468 and the ER+ breast cancer cell lines T47D and MCF7 (FIGS. 3, 5 and 8A). While most reported HDAC PROTACs only degrade HDAC6 or had little effect on HDAC3 at high concentration, the VHL-based PROTACs 8a-d could induce HDAC3 degradation at 100 nM or less in MDA-MB-468, T47D and MCF7 cells despite their compromised HDAC inhibitory activities, whereas these compounds had small or no effects on the protein levels of HDAC1 and HDAC2. The HDAC ligand 15 does not significantly degrade HDAC3 or 2 (FIG. 8A). In contrast, among PROTACs derived from CRBN, only 7c could degrade HDAC3 at 1.0 μM. Notably, HDAC6 degradation can be achieved using either HDAC6 selective inhibitor or pan-HDAC1 as warhead. Our PROTACs are derived from SR-3558, an HDAC1 with a unique benzoylhydrazide ZBG that preferentially binds to HDAC3, which enables efficient degradation of this isoform. In our case, VHL-recruiting degraders appeared to be more potent and selective in inducing HDAC3 degradation, and PROTAC 8c (XZ9002) was the best candidate among its analogs (FIGS. 3 and 5). Thus, we decided to further characterize this compound.

XZ9002 dose-dependently induced HDAC3 degradation in MDA-MB-468 cells, with a DC₅₀ value (the concentration for 50% protein degradation) of 42 nM under 14 h treatment (FIG. 3A). Other compounds with HDAC3 DC₅₀ values of less than 0.1 μM are shown in Table 2. In contrast, no significant changes in the protein levels of HDACs 1, 2, and 6 were observed under the same condition. Pre-incubation of MDA-MB-468 cells with an excess of VHL ligand VH032 (10 μM) or a proteasome inhibitor MG132 blocked XZ9002-induced HDAC3 degradation (FIG. 3B), indicating that the PROTAC-mediated degradation depends on both VHL E3 ligase and the ubiquitin proteasome system (UPS). To further confirm that VHL E3 ligase is involved in XZ9002-induced HDAC3 degradation, we synthesized XZ9002-NC as a negative control compound, in which two chiral centers in VHL binding motif of XZ9002 are reversed to abolish the interaction with VHL E3 ligase. As expected, XZ9002-NC did not induce HDAC3 degradation in MDA-MB-468 cells (FIG. 3B). Besides, the HDAC3 degradation induced by XZ9002 in MDA-MB-468 was time-dependent, starting within 2 h and after drug treatment for 8 h, 70% HDAC3 was degraded with 125 nM of XZ9002 (FIG. 3C). The effects of XZ9002 on HDAC3 protein levels in MDA-MB-468 were long-lasting and reversible. As indicated in the “washout” assay (FIG. 3D), it took more than 12 h for HDAC3 to rebound to the steady level. XZ9002 dose-dependently increased histone acetylation albeit with only moderate effects compared to 15 and CRNB-based PROTACs (FIGS. 3A and 5).

TABLE 2
HDAC3 degradation potency of XZ9002 analogs in T47D cells

| Compound | DC₅₀ (μM) |
|---|---|
| 27a | <1.0 |
| 27b | <0.1 |
| 27c | <0.1 |
| 28a | <0.1 |
| 28b | <0.1 |
| 28c | <0.1 |
| 28d | <0.1 |
| 28e | <1.0 |

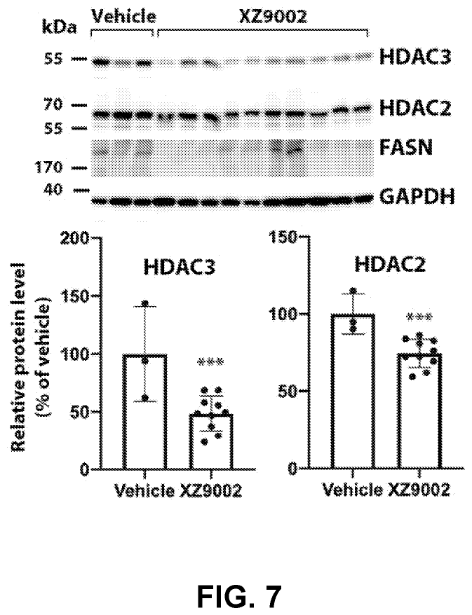

XZ9002 can effectively degrade HDAC3 and to a less extent, HDAC2 in xenograft tumor in female NSG mice bearing MDA-MB-231 xenografts in mammary fat pads (FIG. 7). These data indicate that XZ9002 exhibits the expected pharmacology in vivo and achieves HDAC3 effective degradation in tumors, supporting in vivo dosing of this class of PROTACs for treating human cancers.

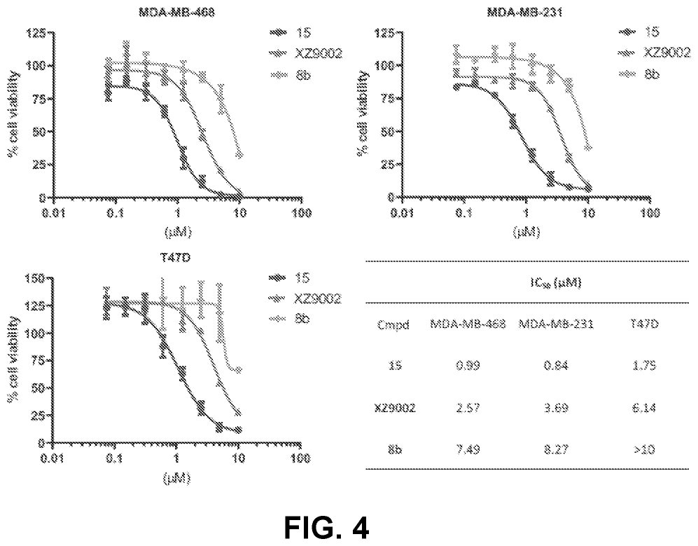

HDACs 1-3 are frequently overexpressed in various cancer types. HDAC3 exhibits biological functions distinct from HDAC1 and HDAC2. HDAC3 requires a cofactor such as NCOR2 for its catalytic activity. It is involved in DNA replication and DNA damage responses. Inhibition of HDAC3 induces DNA replication stress in cutaneous T cell lymphomas. Several studies have suggested that HDAC3 may be a critical factor for breast cancer metastatic progression. In an analysis of clinical breast cancer samples, it has been shown that HDAC3 is highly expressed in tumors with features of aggressive subtypes and that HDAC3 expression levels inversely correlate with patient survival rate. Inhibition of HDAC3 attenuates breast cancer cell proliferation and suppresses the expression of genes underlying cancer stem cell phenotypes. Encouragingly, class I selective HDACi provided survival benefits in combination with the aromatase inhibitor exemestane for patients with hormone receptor-positive breast cancers. Notably, HDAC3 also has deacetylase activity-independent function, which could not be blocked by conventional HDACi. These studies suggest that HDAC3 inhibition can induce distinct cellular response and likely exerts underappreciated anticancer effects. We next evaluated the effects of XZ9002 on the viability of breast cancer cell lines along with 15 and PROTAC 8b. As shown in FIG. 4, HDACi 15 showed potent cytotoxicity against MDA-MB-468 cell line with an IC₅₀ value of 0.99 μM. PROTAC 8b displayed decreased antiproliferative activity, which is likely due to compromised HDAC inhibitory activity. In contrast, XZ9002 with similar HDAC binding profile was more potent in killing cancer cells, suggesting HDAC3 degradation impairs cancer cell survival. Similar trends have been observed in MDA-MB-231 and T47D cells. Clonogenic growth assays were used to further assess the antiproliferative effects of XZ9002 and analogs. XZ9002 is more potent to suppress colony formation of breast cancer cell lines MCF7, T47D, MDA-MB-468, HCC-1143 and BT479 than XZ9002-NC (FIG. 8B), indicating that HDAC3 degradation is important for the anti-proliferative effects of XZ9002. Compound 28c is also effective to inhibit clonogenic growth of the non-small cell line H1299 (FIG. 9). These data collectively highlight a translational potential for HDAC3 degraders in cancer treatment.

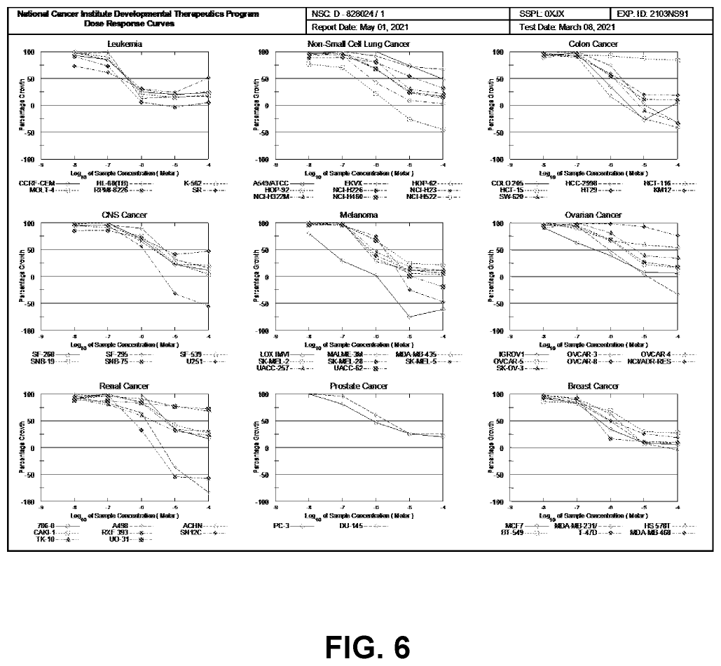

More broadly, XZ9002 effectively inhibits the proliferation of diverse cancer types including leukemia, non-small cell lung cancer, colon cancer, central nervous system cancer, melanoma, ovarian cancer, renal cancer, prostate cancer and breast cancer in a study using the NCI-60 panel of 60 cancer lines (FIG. 6). These data show that XZ9002 and analogs may be applicable for treating diverse cancer types.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A compound having a structure represented by Formula I or a pharmaceutically acceptable salt, solvate, or polymorph thereof: 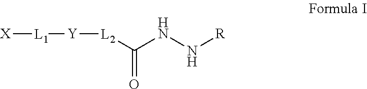
wherein X comprises an E3 ligase targeting moiety;
wherein L₁ comprises a C2-C12 alkyl group; 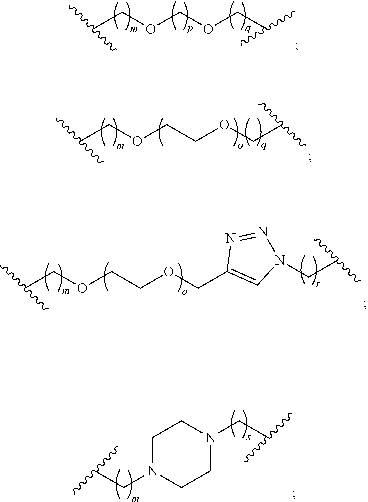
or a combination thereof;
wherein m is from 1 to 11;
is from 0 to 10;
p is from 2 to 4;
q is from 1 to 4;
r is from 0 to 10; and
s is from 1 to 10;
wherein Y comprises 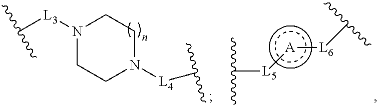
or a combination thereof;
wherein L₃ is omitted or comprises a keto group, an amide group, a sulfonyl group, or a combination thereof;
L₄ is omitted or comprises a keto group, a sulfonyl group, a C1-C2 alkyl group, —C(O)CH₂—, —CH═CH—, or a combination thereof;
n is from 1 to 3;
A comprises a substituted or unsubstituted monocyclic aryl group, a substituted or unsubstituted monocyclic heteroaryl group; or a combination thereof;
L₅ is omitted or comprises an amide group, a sulfonamide group; a keto group; oxygen; —CH═CH—; —CH₂C(O)—NH—; or a combination thereof; and
L₆ is omitted or comprises oxygen, a keto group, an amide group, a sulfonamide group, or a combination thereof;
wherein L₂ comprises a monocyclic aryl group, monocyclic heteroaryl group, or a combination thereof;
and wherein R comprises a substituted or unsubstituted C1-C6 linear or branched alkyl group, a C3-C6 substituted or unsubstituted cycloalkyl group, or a combination thereof.

2. The compound of claim 1, wherein the E3 ligase targeting moiety comprises 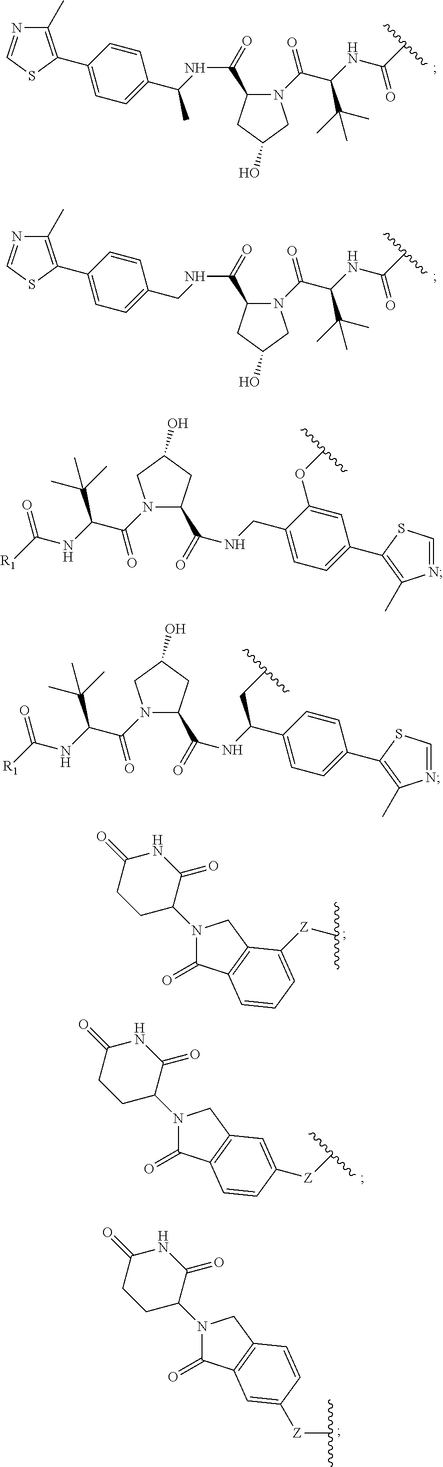 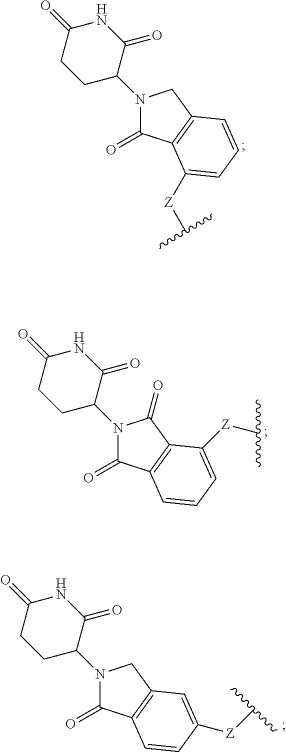
or a combination thereof;
wherein R₁ comprises methyl, 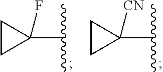
or a combination thereof; and
wherein Z comprises oxygen, NH, methylene, or a combination thereof.

3. The compound of claim 1, wherein the E3 ligase targeting moiety comprises

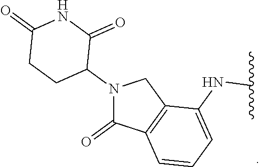 [SMILES: *Nc1cccc2c1CN(C1CCC(=O)NC1=O)C2=O]

4. The compound of claim 1, wherein the E3 ligase targeting moiety comprises

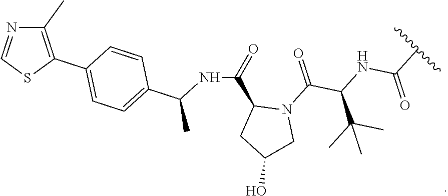 [SMILES: *C(=O)N[C@H](C(=O)N1C[C@H](O)C[C@H]1C(=O)N[C@@H](C)c1ccc(-c2scnc2C)cc1)C(C)(C)C]

5. The compound of claim 1, wherein L₁ comprises a C2-C8 alkyl group; 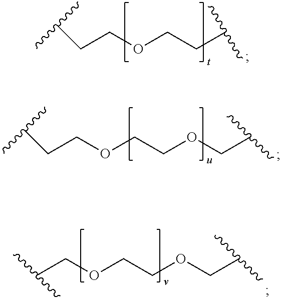
or a combination thereof; and
wherein t, u, and v are independently from 0 to 6.

6. The compound of claim 1, wherein L₁ comprises a C2 alkyl group, C4 alkyl group, a C6 alkyl group, or a C4 alkyl group.

7. The compound of claim 1, wherein L₁ comprises 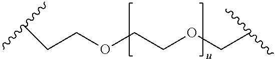
and u is 1, 2, or 3.

8. The compound of claim 1, wherein L₁ comprises 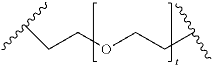
and t is 1, 2, or 3.

9. The compound of claim 1, wherein L₂ comprises a monocyclic aryl group.

10. The compound of claim 1, wherein L₂ comprises

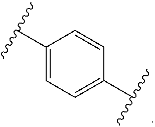 [SMILES: *c1ccc(*)cc1]

11. The compound of claim 1, wherein R comprises a propyl group.

12. The compound of claim 1, having a structure represented by a formula: 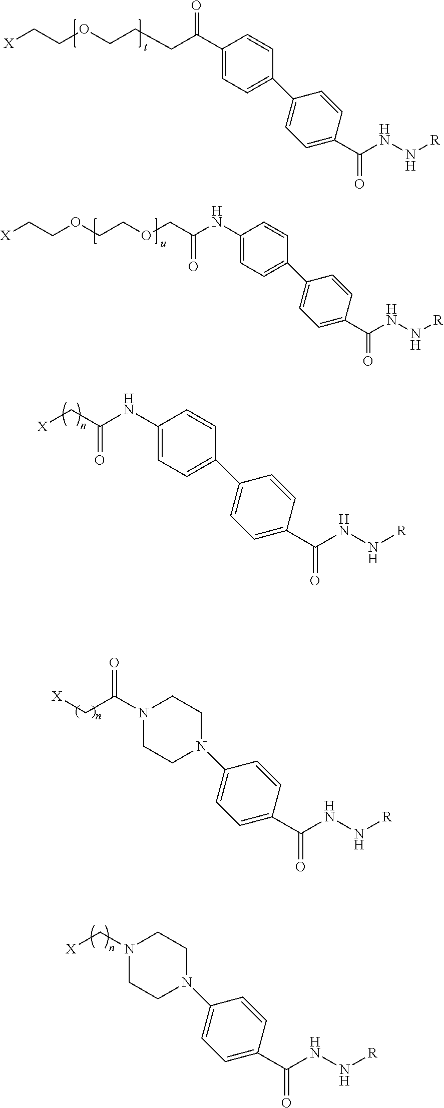
wherein
X is the E3 ligase targeting moiety;
t is 1, 2, or 3;
u is 1, 2, or 3; and
n is an integer from 1 to 10.

13. The compound of claim 12, wherein R is a propyl group.

14. The compound of claim 12, wherein X is

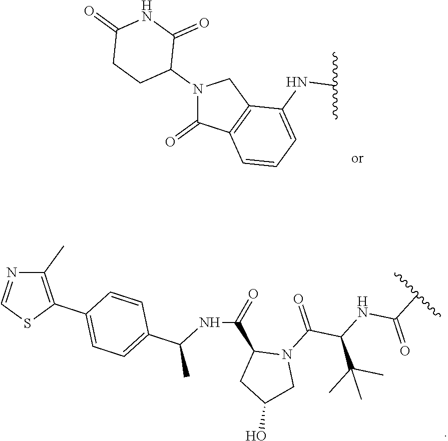 [SMILES: *Nc1cccc2c1CN(C1CCC(=O)NC1=O)C2=O ; *C(=O)N[C@H](C(=O)N1C[C@H](O)C[C@H]1C(=O)N[C@@H](C)c1ccc(-c2scnc2C)cc1)C(C)(C)C]

15. The compound of claim 1, having a structure represented by a formula:

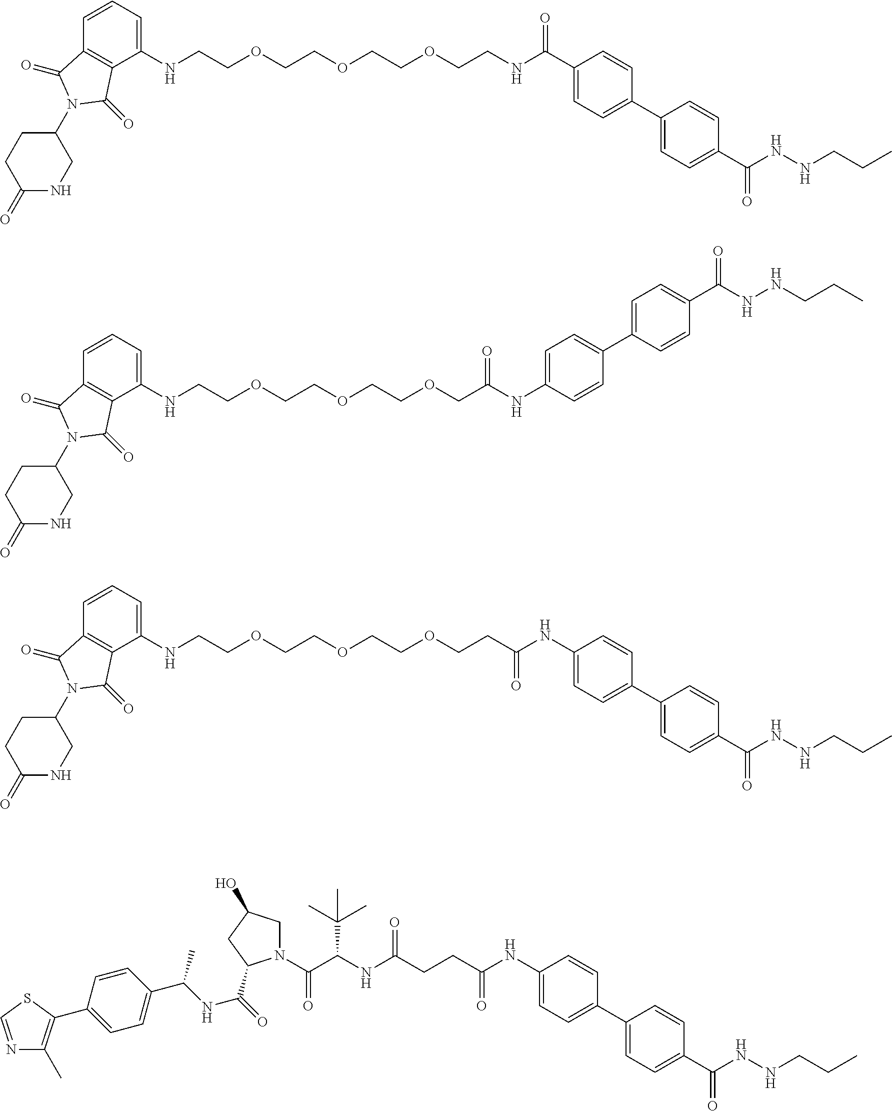 [SMILES: CCCNNC(=O)c1ccc(-c2ccc(C(=O)NCCOCCOCCOCCNc3cccc4c3C(=O)N(C3CCC(=O)NC3)C4=O)cc2)cc1 ; CCCNNC(=O)c1ccc(-c2ccc(NC(=O)COCCOCCOCCNc3cccc4c3C(=O)N(C3CCC(=O)NC3)C4=O)cc2)cc1 ; CCCNNC(=O)c1ccc(-c2ccc(NC(=O)CCOCCOCCOCCNc3cccc4c3C(=O)N(C3CCC(=O)NC3)C4=O)cc2)cc1 ; CCCNNC(=O)c1ccc(-c2ccc(NC(=O)CCC(=O)N[C@H](C(=O)N3C[C@H](O)C[C@H]3C(=O)N[C@@H](C)c3ccc(-c4scnc4C)cc3)C(C)(C)C)cc2)cc1]

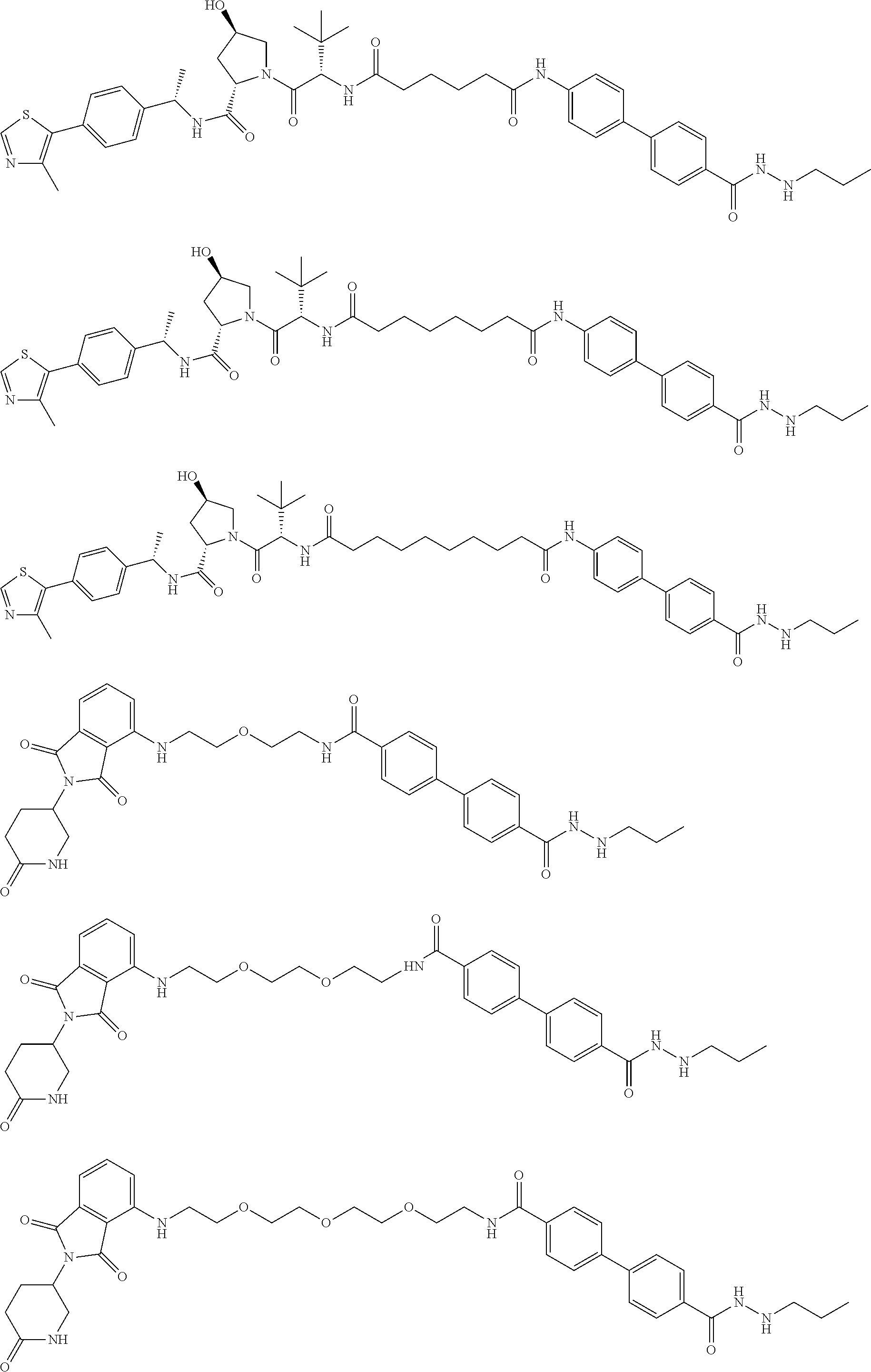

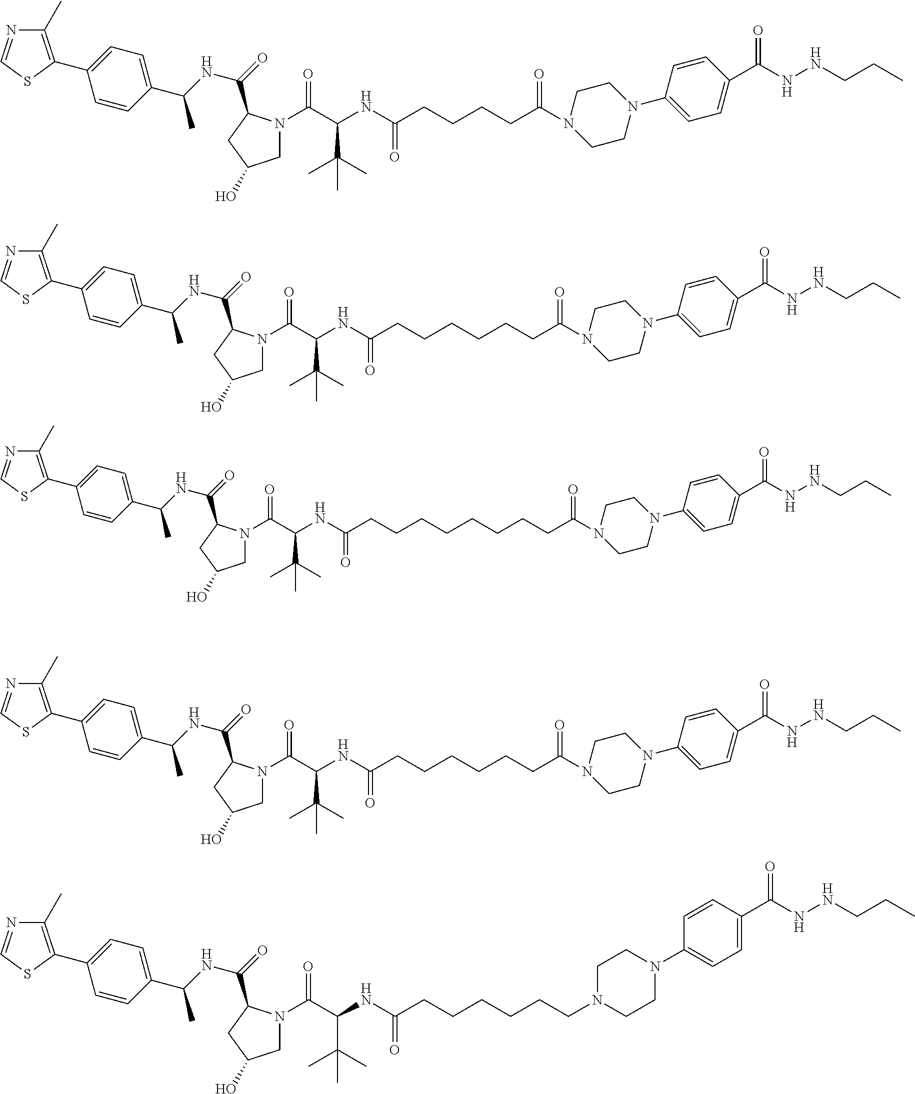

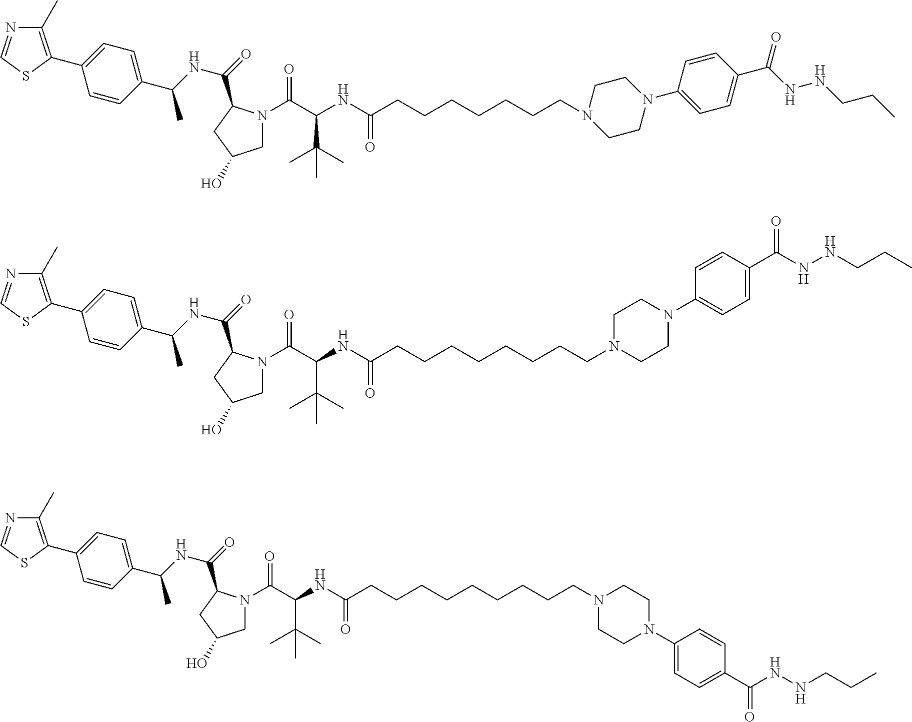 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1 ; CCCNNC(=O)c1ccc(N2CCN(CCCCCCCCC(=O)N[C@H](C(=O)N3C[C@H](O)C[C@H]3C(=O)N[C@@H](C)c3ccc(-c4scnc4C)cc3)C(C)(C)C)CC2)cc1 ; CCCNNC(=O)c1ccc(N2CCN(CCCCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

16. A pharmaceutical composition comprising a therapeutically effective amount of a compound of claim 1, or a pharmaceutically acceptable salt, solvate, or polymorph thereof, and a pharmaceutically acceptable carrier.

17. A method for the treatment of a disorder in a mammal, comprising the step of administering to the mammal a therapeutically effective amount of at least one compound of claim 1.

18. The method of claim 17, wherein the disorder is selected from breast cancer, Hodgkin lymphoma, acute myeloid leukemia, myelodysplastic syndrome, pancreatic cancer, colorectal cancer, ovarian cancer, lung cancer, stomach cancer, muscle cancer, bone cancer, melanoma, bladder cancer, thyroid cancer, liver cancer, glioma, head and neck cancer, renal cancer, urothelial cancer, prostate cancer, testicular cancer, cervical cancer, endometrial cancer, another solid tumor, type 2 diabetes, adipose tissue inflammation, excessive hepatic lipid accumulation, lipodystrophy, insulin resistance or another metabolic disorder, Alzheimer's disease, Parkinson's disease, Huntington's disease, multiple sclerosis, Frederich's ataxia, amyotrophic lateral sclerosis, or another neurodegenerative disease, a neurological disease, rheumatoid arthritis, asthma, chronic obstructive pulmonary disease, cystic fibrosis, acute respiratory distress syndrome, interstitial fibrosis, or another inflammatory disorder, heart disease, stroke, another cardiovascular disease, or a combination thereof.

19. A method for inhibiting the activity of at least one histone deacetylase enzyme in a mammal, comprising the step of administering to the mammal a therapeutically effective amount of at least one compound of claim 1.

20. The method of claim 19, wherein the histone deacetylase enzyme is histone deacetylase 3 (HDAC3).
